SEQUENCE LISTING (64 sequences)

SEQ ID NO: 1 - protein, 39 aa, Heloderma horridum
HSDGTFTSDLSKQMEEEAVRLFIEWLKNGGPSSGAPPPS

SEQ ID NO: 2 - protein, 39 aa, Heloderma suspectum
HGEGTFTSDLSKQMEEEAVRLFIEWLKNGGPSSGAPPPS

SEQ ID NO: 3 - protein, 37 aa, Homo sapiens
HDEFERHAEGTFTSDVSSYLEGQAAKEFIAWLVKGRG

SEQ ID NO: 4 - protein, 585 aa, Homo sapiens [sequence omitted]

SEQ ID NO: 5 - protein, 616 aa, Artificial Sequence
  Description of Artificial Sequence Synthetic construct
HVEGTFTSDVSSYLEGQAAKEFIAWLVKGRGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - protein, 631 aa, Artificial Sequence
  Description of Artificial Sequence Synthetic construct
HVEGTFTSDVSSYLEGQAAKEFIAWLVKGRGGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 7 - protein, 640 aa, Artificial Sequence
  Description of Artificial Sequence Synthetic construct
HGEGTFTSDVSSYLEEQAAKEFIAWLVKGRGSSGAPPPSGGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 624 aa, Homo sapiens
HGEGTFTSDLSKQMEEEAVRLFIEWLKNGGPSSGAPPPSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 9 - protein, 264 aa, Artificial Sequence
  Description of Artificial Sequence Synthetic construct
HVEGTFTSDVSSYLEGQAAKEFIAWLVKGRGAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 272 aa, Artificial Sequence
  Description of Artificial Sequence Synthetic construct
HVEGTFTSDVSSYLEGQAAKEFIAWLVKGRGSSGAPPPSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKETVDKSRWQQGNVFSCSVMHEALHNHYTQKSLSLSPGK

SEQ ID NO: 11 - protein, 272 aa, Artificial Sequence
  Description of Artificial Sequence Synthetic construct
HGEGTFTSDLSKQMEEEAVRLFIEWLKNGGPSSGASSGAAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 39 aa, Artificial Sequence
  Description of Artificial Sequence Synthetic construct
XXXGTXXXXXSKQXEEEAVRLXXXXLKNGGXSSGAXXXX

SEQ ID NO: 13 - protein, 38 aa, Artificial Sequence
  Description of Artificial Sequence Synthetic construct
XXXGXXXXXXXXXXXXXAXXXXXXXXXXGGXSSGAXXX

SEQ ID NO: 14 - protein, 5 aa, Artificial Sequence
  Description of Artificial Sequence Synthetic peptide
GGXSS

SEQ ID NO: 15 - protein, 6 aa, Artificial Sequence
  Description of Artificial Sequence Synthetic peptide
GGXSSG

SEQ ID NO: 16 - protein, 7 aa, Artificial Sequence
  Description of Artificial Sequence Synthetic peptide
GGXSSGA

SEQ ID NO: 17 - protein, 8 aa, Artificial Sequence
  Description of Artificial Sequence Synthetic peptide
GGXSSGAX

SEQ ID NO: 18 - protein, 9 aa, Artificial Sequence
  Description of Artificial Sequence Synthetic peptide
GGXSSGAXX

SEQ ID NO: 19 - protein, 10 aa, Artificial Sequence
  Description of Artificial Sequence Synthetic peptide
GGXSSGAXXX

SEQ ID NO: 20 - protein, 5 aa, Artificial Sequence
  Description of Artificial Sequence Synthetic peptide
GGXSS

SEQ ID NO: 21 - protein, 6 aa, Artificial Sequence
  Description of Artificial Sequence Synthetic peptide
GGXSSG

SEQ ID NO: 22 - protein, 7 aa, Artificial Sequence
  Description of Artificial Sequence Synthetic peptide
GGXSSGA

SEQ ID NO: 23 - protein, 8 aa, Artificial Sequence
  Description of Artificial Sequence Synthetic peptide
GGXSSGAX

SEQ ID NO: 24 - protein, 9 aa, Artificial Sequence
  Description of Artificial Sequence Synthetic peptide
GGXSSGAXX

SEQ ID NO: 25 - protein, 10 aa, Artificial Sequence
  Description of Artificial Sequence Synthetic peptide
GGXSSGAXXX

SEQ ID NO: 26 - protein, 39 aa, Artificial Sequence
  Description of Artificial Sequence Synthetic construct
XXXXXXXXXXXXXXXXXAXXXXXXXXXXGGXSSGAXXXX

SEQ ID NO: 27 - protein, 11 aa, Artificial Sequence
  Description of Artificial Sequence Synthetic peptide
GGXSSGAXXXX

SEQ ID NO: 28 - protein, 4 aa, Artificial Sequence
  Description of Artificial Sequence Synthetic peptide
DAHK

SEQ ID NO: 29 - protein, 39 aa, Artificial Sequence
  Description of Artificial Sequence Synthetic construct
XXEGTFTSDXSXXXEXXAXXXFIXWLXXXXXXXXXXXXX

SEQ ID NO: 30 - protein, 5 aa, Artificial Sequence
  Description of Artificial Sequence Synthetic peptide
KKKKK

SEQ ID NO: 31 - protein, 39 aa, Artificial Sequence; identical to SEQ ID NO: 29
  Description of Artificial Sequence Synthetic construct

SEQ ID NO: 32 - protein, 39 aa, Artificial Sequence
  Description of Artificial Sequence Synthetic construct
HXXGXFTXDXXXXXXXXXXXXFIXXXXXXXXXXXXXXXX

SEQ ID NO: 33 - protein, 31 aa, Artificial Sequence
  Description of Artificial Sequence Synthetic construct
HAEGTFTSDVSSYLEGQAAKEFIAWLVRGRG

SEQ ID NO: 34 - protein, 41 aa, Artificial Sequence
  Description of Artificial Sequence Synthetic construct
HXEGTFTSDVSSYLEXQAAKEFHEAWLXKGGPSSGAPPPCC

SEQ ID NO: 35 - protein, 31 aa, Artificial Sequence
  Description of Artificial Sequence Synthetic construct
XXEGTFTSDVSSYLXGQAAKXFIAWLVKGRX

SEQ ID NO: 36 - protein, 30 aa, Artificial Sequence
  Description of Artificial Sequence Synthetic construct
AEGTFTSDVSSYLEGQAAXEFIAWLVKGRX

SEQ ID NO: 37 - protein, 30 aa, Artificial Sequence
  Description of Artificial Sequence Synthetic peptide
GGGGSGGGGSGGGGSGGGGSGGGGSGGGGS

SEQ ID NO: 38 - protein, 15 aa, Artificial Sequence
  Description of Artificial Sequence Synthetic peptide
GGGGSGGGGSGGGGS

SEQ ID NO: 39 - protein, 22 aa, Artificial Sequence
  Description of Artificial Sequence Synthetic peptide
GSGGGGSGGGGSGGGGSGGGGS

SEQ ID NO: 40 - protein, 30 aa, Artificial Sequence; identical to SEQ ID NO: 37
  Description of Artificial Sequence Synthetic peptide

SEQ ID NO: 41 - protein, 25 aa, Artificial Sequence
  Description of Artificial Sequence Synthetic peptide
DAAAKEAAAKDAAAREAAARDAAAK

SEQ ID NO: 42 - protein, 14 aa, Artificial Sequence
  Description of Artificial Sequence Synthetic peptide
NVDHKPSNTKVDKR

SEQ ID NO: 43 - protein, 8 aa, Artificial Sequence
  Description of Artificial Sequence Synthetic peptide
SSGAPPPS

SEQ ID NO: 44 - protein, 8 aa, Artificial Sequence
  Description of Artificial Sequence Synthetic peptide
SSGASSGA

SEQ ID NO: 45 - protein, 6 aa, Artificial Sequence
  Description of Artificial Sequence Synthetic peptide
PPCPSC

SEQ ID NO: 46 - protein, 230 aa, Artificial Sequence
  Description of Artificial Sequence Synthetic construct
XESKYGPPCPPCPAPXXXGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 47 - protein, 230 aa, Artificial Sequence
  Description of Artificial Sequence Synthetic construct
AESKYGPPCPPCPAPXXXGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 48 - protein, 31 aa, Artificial Sequence
  Description of Artificial Sequence Synthetic peptide
HXEGTFTSDVSSYLEEQAAKEFIAWLVKGGG

SEQ ID NO: 49 - protein, 31 aa, Artificial Sequence
  Description of Artificial Sequence Synthetic peptide
HXEGTFTSDVSSYLEEQAAKEFIAWLKNGGG

SEQ ID NO: 50 - protein, 33 aa, Artificial Sequence
  Description of Artificial Sequence Synthetic peptide
HXEGTFTSDVSSYLEEQAIAAKEFIAWLVKGGP

SEQ ID NO: 51 - protein, 31 aa, Artificial Sequence
  Description of Artificial Sequence Synthetic peptide
HXEGTFTSDVSSYLEEQAAKEFIAWLKNGGP

SEQ ID NO: 52 - protein, 30 aa, Artificial Sequence
  Description of Artificial Sequence Synthetic peptide
HXEGTFTSDVSSYLEEQAAKEFIAWLVKGG

SEQ ID NO: 53 - protein, 30 aa, Artificial Sequence
  Description of Artificial Sequence Synthetic peptide
HXEGTFTSDVSSYLEEQAAKEFIAWLKNGG

SEQ ID NO: 54 - protein, 30 aa, Heloderma suspectum
HGEGTFTSDLSKQMEEEAVRLFIEWLKNGG

SEQ ID NO: 55 - protein, 27 aa, Heloderma suspectum
HGEGTFTSDLSKQMEEEAVRLFIEWLK

SEQ ID NO: 56 - protein, 28 aa, Heloderma suspectum
HGEGTFTSDLSKQMEEEAVRLFIEWLKN

SEQ ID NO: 57 - protein, 39 aa, Artificial Sequence
  Description of Artificial Sequence Synthetic peptide
HGEGTFTSDLSKQLEEEAVRLFIEFLKNGGPSSGAPPPS

SEQ ID NO: 58 - protein, 39 aa, Artificial Sequence
  Description of Artificial Sequence Synthetic peptide
HGEGTFTSDLSKQLEEEAVRLFIEWLKNGGPSSGAPPPS

SEQ ID NO: 59 - protein, 28 aa, Artificial Sequence
  Description of Artificial Sequence Synthetic peptide
HGEGTFTSDLSKQLEEEAVRLFIEFLKN

SEQ ID NO: 60 - protein, 28 aa, Artificial Sequence
  Description of Artificial Sequence Synthetic peptide
HGEGTFTSDLSKQLEEEAVRLFIEWLKN

SEQ ID NO: 61 - protein, 39 aa, Artificial Sequence
  Description of Artificial Sequence Synthetic peptide
HGEGTFTSDLSKQMEEEAVXLFIEWLKNGGPSSGAPPPS

SEQ ID NO: 62 - protein, 39 aa, Artificial Sequence
  Description of Artificial Sequence Synthetic peptide
HGEGTFTSDLSKQMEEEAVRLFIEWLKNGGPXSGAPPPS

SEQ ID NO: 63 - protein, 40 aa, Artificial Sequence
  Description of Artificial Sequence Synthetic peptide
HGEGTFTSDLSKQMEEEAVRLFIEWLKNGGPSSGAPPPSX

SEQ ID NO: 64 - protein, 40 aa, Artificial Sequence; identical to SEQ ID NO: 63
  Description of Artificial Sequence Synthetic peptide

(19) United States
(12) Patent Application Publication
(10) Pub. No.: US 2009/0209469 A1
(43) Pub. Date: Aug. 20, 2009

(54) Use of Exendins and GLP-1 Receptor Agonists for Altering Lipoprotein Particle Size and Subclass Composition

(76) Inventors: Dennis Kim, La Jolla, CA (US); Michael Trautmann, Hamburg (DE); Don D. Johns, San Antonio, IN (US); David Robbins, Weatherley Lake, MO (US)

Correspondence Address: Intellectual Property Department;Amylin Pharmaceuticals, Inc., 9360 Towne Centre Drive, San Diego, CA, 92121, US

(21) Appl. No.: 12/374,569

(22) PCT Filed: Aug. 6, 2007
(86) PCT No.: PCT/US07/17540

§ 371 Date: Apr. 15, 2009

Related U.S. Application Data
(60) Provisional application No. 60/835,435, filed on Aug. 4, 2006.

(51) Int. Cl. A61K 38/17; A61K 38/22; A61P 9/00

(52) U.S. Cl. 514/12

(57) ABSTRACT

The present invention relates to altering the concentration of various lipid molecules, specifically for example by shifting from small LDL particles to large LDL and HDL particles. The present invention also relates to methods for increasing average lipid particle size and methods for improving the cardiovascular risk profile of a subject by altering lipid particle sizes or concentrations or both.

### CROSS-REFERENCE TO RELATED PATENT APPLICATIONS

This application claims the benefit of U.S. Provisional Application No. 60/835,435, filed Aug. 4, 2006, entitled “Use of Exendins and Exendin Agonists for Altering Lipoprotein Particle Size and Subclass Composition”, which is incorporated herein by reference in its entirety and for all purposes.

### FIELD OF THE INVENTION

Disclosed herein are methods relating to altering the concentration of various lipid molecules, for example without limitation by shifting from small LDL particles to large LDL and HDL particles. Also disclosed are methods for increasing average lipid particle size and methods for improving the cardiovascular risk profile of a subject by altering lipid particle sizes or concentrations or both.

### BACKGROUND OF THE INVENTION

HDL is a high density class of lipoproteins that for example carry cholesterol from body tissues to the liver. LDL is a low density class of lipoproteins which for example carry cholesterol in the blood and around the body, for use by cells. Elevated LDL levels or decreased HDL levels or both have been linked to cardiovascular diseases and conditions and other diseases.

Exendins are peptides that were first isolated from the salivary secretions of the Gila monster, a lizard found in Arizona, and the Mexican Beaded Lizard. Exendin-3 (SEQ ID NO:_) is present in the salivary secretions of Heloderma horridum, and exendin-4 (SEQ ID NO:_) is present in the salivary secretions of Heloderma suspectum (Eng., J., et al., J. Biol. Chem., 265:20259-62, 1990; Eng., J., et al., J. Biol. Chem., 267:7402-05, 1992).

Glucagon-like peptide 1 (GLP-1) (SEQ ID NO:_) is a product of processing of proglucagon. Circulating biologically active GLP-1 is found in several forms including the GLP-1(7-36) amide and GLP-1(7-37) forms. GLP-1 is secreted from gut endocrine cells in response to nutrient ingestion and plays multiple roles in metabolic homeostasis following nutrient absorption. An important locus for regulation of GLP-1 biological activity is the N-terminal degradation of the peptide by Dipeptidyl Peptidase-4 (DPP-4)-mediated cleavage at position 2 alanine of GLP-1(7-37). By convention in the art, the sequence numbering of GLP-1 and analogs customarily begins with residue 7, corresponding to the N-terminal residue of GLP-1(7-37). Unless indicated otherwise, this convention is followed herein.
Decreasing the concentration of one or more small LDL, increasing the conc7 Yentration of one or more large LDL, or both may reduce the risk of cardiovascular diseases and conditions or other disease states that are associated with increased concentrations of small lipoproteins. Accordingly, there are provided herein therapeutics and methods of use thereof by which, for example without limitation, the concentration of larger lipoproteins may be increased, smaller lipoproteins may be decreased or where average lipoprotein size may be shifted from smaller to larger lipoproteins, such aLP-1 receptor agonists.

### SUMMARY OF THE INVENTION

In a first aspect, provided herein are methods for increasing the concentration of large LDL, large HDL, total HDL or any combination of said lipoproteins in a subject in need thereof comprising administering to said subject a therapeutically effective amount of an exendin, exendin agonist or GLP-1 receptor agonist, wherein the concentration of large LDL, large HDL, total HDL, or any combination of said lipoproteins is increased in said subject.

In another aspect, provided herein are methods for increasing the concentration of large LDL, large HDL, total HDL or any combination of said lipoproteins in a subject who has a decreased large LDL, large HDL, total HDL level, or any combination thereof comprising administering to said subject a therapeutically effective amount of an exendin, exendin agonist or GLP-1 receptor agonist, wherein the concentration of large LDL, large HDL, total HDL, or any combination of said lipoproteins is increased in said subject.

In another aspect, provided herein are methods for decreasing the concentration of small LDL, very small LDL, total LDL or any combination of said lipoproteins in a subject in need thereof comprising administering to said subject a therapeutically effective amount of an exendin, exendin agonist or GLP-1 receptor agonist, wherein the concentration of small LDL is decreased.

In another aspect, provided herein are methods for decreasing the concentration of small LDL, very small LDL, total LDL or any combination of said lipoproteins in a subject who has an elevated level of small LDL, very small LDL, total LDL or any combination thereof comprising administering to said subject a therapeutically effective amount of an exendin, exendin agonist or GLP-1 receptor agonist, wherein the concentration of small LDL is decreased.

In another aspect, provided herein are methods for providing an improved cardiovascular risk profile of a subject in need thereof comprising administering to said subject a therapeutically effective amount of an exendin, exendin agonist or GLP-1 receptor agonist and measuring an increased concentration of large LDL, large HDL, total HDL or any combination of said lipoproteins, wherein the cardiovascular risk profile of said subject is improved.

In another aspect, provided herein are methods for providing an improved cardiovascular risk profile of a subject who has a decreased level of large LDL, large HDL, total HDL or any combination thereof comprising administering to said subject a therapeutically effective amount of an exendin, exendin agonist or GLP-1 receptor agonist and measuring an increased concentration of large LDL, large HDL, total HDL or any combination of said lipoproteins, wherein the cardiovascular risk profile of said subject is improved.

In another aspect, provided herein are methods for treating a subject with an elevated level of small LDL, very small LDL or total LDL or any combination of said lipoproteins, comprising administering to said subject a therapeutically effective amount of an exendin, exendin agonist or GLP-1 receptor agonist, wherein the concentration of small LDL, very small LDL, total LDL or any combination of said lipoproteins is decreased in said subject.

In another aspect, provided herein are methods for increasing the average particle size of LDL or HDL in a subject in need thereof comprising administering to said subject a therapeutically effective amount of an exendin, exendin agonist or GLP-1 receptor agonist, wherein the particle size of LDL or HDL is increased in said subject.

In another aspect, provided herein are methods for increasing the average particle size of LDL or HDL in a subject who has an elevated level of small LDL, a decreased level of large HDL, a decreased level of total HDL or any combination thereof comprising administering to said subject a therapeutically effective amount of an exendin, exendin agonist or GLP-1 receptor agonist, wherein the particle size of LDL or HDL is increased in said subject.

These and other aspects described herein will be more clearly understood with reference to the following embodiments and detailed description.

### DETAILED DESCRIPTION OF THE, INVENTION

The methods described herein relate generally to administration of exendins, exendin agonists or GLP-1 receptor agonists to alter lipoprotein particle sizes, concentrations, or both. The methods relate, for example without limitation, to the administration of one or more exendins, exendin agonists or GLP-1 receptor agonists to shift lipid particle concentration from small LDL particles to large LDL particles, HDL particles, or both. As a further non-limiting example, the present methods relate to increasing the average particle size of one or more lipoprotein. In yet another non-limiting example, the present methods relate to improving the cardiovascular risk profile of a subject by increasing the average particle size of one or more lipoproteins or by shifting lipid particle concentration from small LDL particles to large LDL particles, HDL particles, or both.

In certain embodiments, the altering of lipoprotein concentrations in a subject may include methods of increasing some lipoprotein concentrations and decreasing various other lipoprotein concentrations comprising administering one or more exendins, exendin agonists or GLP-1 receptor agonist. In certain embodiments, administering any one or more exendins, exendin agonists or GLP-1 receptor agonists is contemplated. In certain embodiments, administration of one or more exendins, exendin agonists or GLP-1 receptor agonists is accomplished by any mode of administration known to the skilled artisan. Various exemplary, non-limiting modes of administration are discussed herein.

In certain embodiments, there are provided herein methods of increasing the concentration of large LDL, large HDL, total HDL or any combination of said lipoproteins in a subject in need thereof comprising administering to said subject a therapeutically effective amount of an exendin, exendin agonist or GLP-1 receptor agonist, wherein the concentration of large LDL, large HDL, total HDL, or any combination of said lipoproteins is increased in said subject.

In certain embodiments, there are provided herein methods of increasing the concentration of large LDL, large HDL, total HDL or any combination of said lipoproteins in a subject who has a decreased large LDL, large HDL, total HDL level, or any combination thereof comprising administering to said subject a therapeutically effective amount of an exendin, exendin agonist or GLP-1 receptor agonist, wherein the concentration of large LDL, large HDL, total HDL, or any combination of said lipoproteins is increased in said subject.

In certain embodiments, there are provided herein methods of decreasing the concentration of small LDL, very small LDL, total LDL or any combination of said lipoproteins in a subject in need thereof comprising administering to said subject a therapeutically effective amount of an exendin, exendin agonist or GLP-1 receptor agonist, wherein the concentration of small LDL is decreased.

In certain embodiments, there are provided herein methods of decreasing the concentration of small LDL, very small LDL, total LDL or any combination of said lipoproteins in a subject who has an elevated level of small LDL, very small LDL, total LDL or any combination thereof comprising administering to said subject a therapeutically effective amount of an exendin, exendin agonist or GLP-1 receptor agonist, wherein the concentration of small LDL is decreased.

In certain embodiments, a lipoprotein may be a low density lipoprotein, which may be referred to as an LDL. “LDL” refers to a low density lipoprotein as known to one of skill in the art. In certain embodiments, LDL refers to total LDL, which includes any low density lipoprotein regardless of subclass. In certain embodiments, LDL is a small LDL. In certain embodiments, LDL is a large LDL. In certain embodiments, LDL is a very small LDL.

In various embodiments of the present invention, LDL refers to a low density lipoprotein particle that is about 18 nm, about 19 nm, about 20 nm, about 21 nm, about 22 nm, about 23 nm, about 24 nm, or about 25 nm. In further embodiments, LDL refers to a low density lipoprotein particle that has a size ranging from about 18 nm to about 25 nm, about 19 nm to about 23 nm, about 20 nm to about 22 nm, about 18 nm to about 22 nm, about 18 nm to about 21 nm, about 18 nm to about 20 nm, about 18 nm to about 19 nm, about 19 nm to about 23 nm, or about 20 nm to about 25 nm in diameter, about 21 nm to about 23 nm, about 22 nm to about 23 nm, about 19 nm to about 21 nm, or about 20 nm to about 22 nm in diameter.

In certain embodiments, “large LDL” refers to any LDL known to one of skill in the art to be a large LDL In other embodiments of the present invention, large LDL refers to an LDL that is at least 21.2 nm, about 21.5 nm, about 22 nm, about 22.5 nm, about 23 nm in diameter. In certain embodiments, large LDL refers to an LDL that is at least 21.2 nm to about 23 nm in diameter. In further embodiments, large LDL refers to an LDL that is at least about 21.5 nm to about 23 nm n, at least about 22 nm to about 24 nm, about 22 nm to about 23 nm, about 21.5 nm to about 22.5 nm in diameter.

In other embodiments, “small LDL” refers to LDL that is 19.8 nm, about 20 nm, about 20.2 nm, about 20.4 nm, about 20.6 nm, about 20.8 nm, or less than about 21 nm in diameter. In further embodiments of the present invention, small LDL refers to LDL that is at least 19.8 nm to less than 21.2 nm, at least 20 nm to less than 21 nm, or at least 19.8 nm to about 20.5 nm in diameter.

In other embodiments, “very small LDL” refers to particles having a diameter of about 18 nm, about 18.2 nm, about 18.4 nm, about 18.6 nm, about 18.8 nm, about 19 nm, about 19.2 nm, about 19.4 nm, about 19.6 nm, or less than 19.8 nm. In further embodiments, very small LDL particles have a diameter of about 18 nm to less than 19.8 nm, at least 18.5 nm to less than 19.5 nm, about 18.5 nm to about 19 nm, or about 19 nm to less than 19.5 nm.

In further embodiments, a lipoprotein is a very low density lipoprotein, which may be referred to as a VLDL. In certain embodiments, VLDL refers to total VLDL, which includes any very low density lipoprotein regardless of subclass. In certain embodiments, VLDL refers to a small VLDL.

In certain embodiments, “VLDL” refers to any VLDL known to one of skill in the art to be an VLDL. In various embodiments, a VLDL has a particle diameter of more than about 25 nm, more than about 27 nm, more than about 30 nm, more than about 35 nm, more than about 40 nm, more than about 50 nm, or more than about 60 nm. In other embodiments, a VLDL is about 25 nm to about 75 nm, about 27 nm to more than about 60 nm, about 35 nm to about 60 nm, about 27 nm to about 35 nm, or about 35 nm to about 60 nm in diameter.

In certain embodiments, lipoprotein particle size is measured as the average diameter of particles. By way of non-limiting example, HDL particle size may be measured as the average diameter of HDL particles, and large HDL particle size may be measured as the average diameter of large HDL particles.

In certain embodiments, a lipoprotein may be a high density lipoprotein, which may be referred to as an HDL. In certain embodiments, HDL refers to total HDL, which includes any high density lipoprotein regardless of subclass. In certain embodiments, HDL is a small HDL. In certain embodiments, HDL is a large HDL.

In certain embodiments, “HDL” refers to any HDL known to one of skill in the art to be an HDL. In certain embodiments, HDL refers to a high density lipoprotein particle that is less than about 13 nm. In certain embodiments, HDL is a high density lipoprotein with a diameter of about 5 nm, about 6 nm, about 7 nm, about 8 nm, about 9 nm, about 10 nm, about 11 nm, about 12 nm, about 13 nm, or about 14 nm. In filter embodiments, HDL refers to a high density lipoprotein particle that has a size ranging from about 6 nm to about 13 m-n, about 7 nm to about 12 nm, about 10 nm to about 12 nm, about 8 nm to about 11 nm, about 8 nm to about 10 nm, about 9 nm to about 11 nm, or about 9 n-m to about 10 nm in diameter.

In other embodiments of the present invention, “large HDL” is a high density lipoprotein with a diameter of about 8.5 nm, about 9 nm, about 9.5 nm, about 10 nm, about 10.5 nm, about 11 nm, about 11.5 nm, about 12 nm, about 12.5 nm, or about 13 nm. In certain embodiments, large HDL has a diameter of at least 8.5 nm to about 13 nm. In other embodiments, large HDL is a high density lipoprotein with a diameter of about 9.5 nm to about 12 nm, about 10 nm to about 11.5 nm, about 10 nm to about 12.5 nm, or at least about 9 nm to about 11.5 nm.

In further embodiments of the present invention, “small HDL” is a high density lipoprotein with a diameter of about 7 nm, about 7.5 nm, about 8 nm, or less than 8.5 nm. In certain embodiments, small HDL has a diameter of about 7.3 nm to less than 8.5 nm. In further embodiments, small HDL is about 7.3 nm to less than 8.5 nm, about 7.5 nm to less than 8.5 nm, about 8 nm to about 8.5 nm, about 7 nm to about 7.5 nm, about 7.3 nm to less than about 8.2 nm, or about 7.8 nm to less than 8.5 nm in diameter.

In certain embodiments, the concentration of one or more lipoproteins selected from the group consisting of large LDL, large HDL, or total HDL is increased in a subject in need of such an increase in lipoprotein concentration. In certain embodiments, the concentration of large LDL is increased in a subject. In certain embodiments, the concentration of large HDL is increased in a subject. In certain embodiments, the concentration of total HDL is increased in a subject. In certain embodiments, the concentration of two or more lipoproteins selected from the group consisting of large LDL, large HDL, or total HDL are increased in a subject. In certain embodiments, the concentration of large LDL, large HDL, and total HDL are increased in a subject.

In certain embodiments, one or more lipoproteins selected from the group consisting of very small LDL, small LDL, total LDL, small VLDL and total VLDL is decreased in a subject. In certain embodiments, the concentration of two or more lipoproteins selected from the group consisting of very small LDL, small LDL, total LDL, small VLDL and total VLDL is decreased in a subject. In certain embodiments, the concentration of three or more lipoproteins selected from the group consisting of very small LDL, small LDL, total LDL, small VLDL and total VLDL is decreased in a subject. In certain embodiments, the concentration of four or more lipoproteins selected from the group consisting of very small LDL, small LDL, total LDL, small VLDL and total VLDL is decreased in a subject. In certain embodiments, the concentration of very small LDL, small LDL, total LDL, small VLDL and total VLDL is decreased in a subject.

In certain embodiments, one or more lipoproteins selected from the group consisting of very small LDL, small LDL, and total LDL is decreased in a subject. In certain embodiments, two or more lipoproteins selected from the group consisting of very small LDL, small LDL, and total LDL are decreased in a subject. In certain embodiments, very small LDL, small LDL, and total LDL are decreased in a subject.

In certain embodiments, the concentration of one or more lipoproteins selected from the group consisting of large LDL, large HDL, or total HDL is increased in a subject and the concentration of one or more other lipoproteins selected from the group consisting of very small LDL, small LDL, total LDL, small VLDL and total VLDL are decreased. In various embodiments, large LDL, large HDL, total HDL, or any combination of such lipoproteins may be increased and very small LDL, small LDL, total LDL, small VLDL, total VLDL, or any combinations thereof are decreased.

In certain embodiments, the concentration of large LDL, large HDL, or total HDL is increased and average LDL particle size, average HDL particle size, or both are increased. In certain embodiments, the concentration of one or more lipoproteins selected from the group consisting of large LDL, large HDL, and total HDL is increased, and the concentration of one or more lipoproteins selected from the group consisting of very small LDL, small LDL, total LDL, small VLDL, and total VLDL is decreased, and the average LDL particle size, average HDL particle size or both are increased.

In certain embodiments, the concentration of large LDL is increased in a subject by administering an exendin, exendin agonist or GLP-1 receptor agonist. In certain embodiments, an increase in the concentration of large LDL may be an increase of any amount.

In still other embodiments, the concentration of large LDL is increased by less than about 40 nmol/L, by about 40 nmol/L, about 50 nmol/L, about 60 nmol/L, about 70 nmol/L, about 80 nmol/L, about 90 nmol/L, about 100 nmol/L, about 10 nmol/L, about 120 nmol/L, about 130 nmol/L, about 140 nmol/L, or by more than about 140 nmol/L. In other embodiments, the concentration of large LDL is increased by about 40 nmol/L to about 140 nmol/L, about 50 nmol/L to about 120 nmol/L, about 60 nmol/L to about 120 nmol/L, about 60 nmol/L to about 100 nmol/L, about 80 mmol/L to about 100 nmol/L, or about 60 nmol/L to about 80 nmol/L.

In certain embodiments, the concentration of large HDL is increased in a subject by administering an exendin, exendin agonist or GLP-1 receptor agonist. In certain embodiments, an increase in the concentration of large HDL may be an increase of any amount.

In still other embodiments, the concentration of large HDL is increased by less than about 0.4 μmol/L, by about 0.4 μmol/L, about 0.5 μmol/L, about 0.6 μmol/L, about 0.7 μmol/L, about 0.8 μmol/L, about 0.9 μmol/L, about 1.0 μmol/L, about 1 μmol/L, about 1.2 μmol/L, about 1.3 μmol/L, about 1.4 μmol/L, or by more than about 1.4 μmol/L. In other embodiments, the concentration of large HDL is increased by about 0.4 μmol/L to about 1.4 μmol/L, about 0.5 μmol/L to about 1 μmol/L, about 0.6 μmol/L to about 1.2 μmol/L, 0.8 μmol/L to about 1.0 μmol/L, about 0.6 μmol/L to about 0.8 μmol/L.

In certain embodiments, the concentration of total HDL is increased in a subject by administering an exendin, exendin agonist or GLP-1 receptor agonist. In certain embodiments, an increase in the concentration of total HDL may be an increase of any amount.

In still other embodiments, the concentration of total HDL is increased by less than about 0.1 μmol/L, about 0.1 μmol/L, about 0.2 μmol/L, about 0.3 μmol/L, about 0.4 μmol/L, about 0.5 μmol/L, about 0.6 μmol/L, or about 0.7 μmol/L. In other embodiments, the concentration of total HDL is increased by about 0.1 μmol/L to about 0.7 μmol/L, about 0.2 μmol/L to about 0.6 μmol/L, about 0.3 μmol/L to about 0.5 μmol/L, about 0.8 μmol/L to about 1.0 μmol/L, about 0.6 μmol/L to about 0.8 μmol/L, or about 0.3 μmol/L to about 0.4 μmol/L. In certain embodiments, the concentration of large LDL is increased by about 40 nmol/L to about 140 mmol/L, the concentration of large HDL is increased by about 0.40 μmol/L to about 1.4 μmol/L, and the concentration of total HDL is increased by about 0.1 μmol/L to about 0.7 μmol/L. In certain embodiments, the concentration of large LDL is increased by about 50 nmol/L to about 120 nmol/L, the concentration of large HDL is increased by about 0.50 μmol/L to about 1 μmol/L, and the concentration of total HDL is increased by about 0.2 μmol/L to about 0.6 μmol/L. In certain embodiments, the concentration of large LDL is increased by about 60 nmol/L to about 100 nmol/L, the concentration of large HDL is increased by about 0.60 μmol/L to about 0.8 μmol/L, and the concentration of total HDL is increased by about 0.3 μmol/L to about 0.5 μmol/L.

In certain embodiments, the concentration of very small LDL is decreased in a subject by administering an exendin, exendin agonist or GLP-1 receptor agonist. In certain embodiments, a decrease in the concentration of very small LDL may be a decrease of any amount. In various embodiments, the concentration of very small LDL is decreased by less than about 110 nmol/L, by about 110 nmol/L, about 120 nmol/L, about 130 nmol/L, about 140 nmol/L, about 150 nmol/L, about 160 nmol/L, about 170 nmol/L, about 180 nmol/L, about 200 nmol/L, about 220 nmol/L, about 240 nmol/L, or by more than about 240 mmol/L.

In certain embodiments, the concentration of very small LDL is decreased by about 110 nmol/L to about 240 nmol/L, about 115 nmol/L to about 200 nmol/L, about 120 nmol/L to about 180 nmol/L, about 110 nmol/L to about 150 nmol/L, about 120 nmol/L to about 130 nmol/L, about 120 nmol/L to about 140 nmol/L, about 10 nmol/L to about 140 nmol/L or about 110 nmol/L to about 130 nmol/L.

In certain embodiments, the concentration of small LDL is decreased in a subject by administering an exendin, exendin agonist or GLP-1 receptor agonist. In certain embodiments, a decrease in the concentration of small LDL may be a decrease of any amount. In various embodiments, the concentration of small LDL is decreased by less than about 120 nmol/L, by about 120 nmol/L, about 130 nmol/L, about 140 nmol/L, about 150 nmol/L, about 160 nmol/L, about 170 nmol/L, about 180 nmol/L, about 190 nmol/L, about 200 nmol/L, about 220 nmol/L, about 240 nmol/L, or by more than about 240 nmol/L.

In certain embodiments, the concentration of small LDL is decreased by about 120 nmol/L to about 300 nmol/L, about 130 nmol/L to about 250 nmol/L, about 130 nmol/L to about 200 mmol/L, about 140 nmol/L to about 250 nmol/L, about 140 nmol/L to about 200 nmol/L, about 150 nmol/L to about 250 nmol/L, about 150 nmol/L to about 200 nmol/L, about 160 mmol/L to about 250 mmol/L, about 160 nmol/L to about 200 mmol/L, about 130 nmol/L to about 160 nmol/L, about 130 nmol/L to about 150 nmol/L, about 140 nmol/L to 150 nmol/L, about 130 nmol/L to about 160 nmol/L, or about 140 nmol/L to about 160 nmol/L.

In certain embodiments, the concentration of total LDL is decreased in a subject by administering an exendin, exendin agonist or GLP-1 receptor agonist. In certain embodiments, a decrease in the concentration of total LDL may be a decrease of any amount. In various embodiments, the concentration of total LDL is decreased by for example without limitation less than about 60 nmol/L, by about 60 mmol/L, about 70 nmol/L, about 80 nmol/L, about 90 nmol/L, about 100 nmol/L, about 110 nmol/L, about 120 nmol/L, about 140 nmol/L, about 160 nmol/L, about 180 nmol/L, or by more than about 180 nmol/L.

In other embodiments total LDL concentration is decreased by about 40 nmol/L to about 125 nmol/L, about 60 nmol/L to about 180 nmol/L, about 50 nmol/L to about 180 nmol/L, about 50 nmol/L to about 150 nmol/L, about 60 nmol/L to about 120 nmol/L, about 70 nmol/L to about 110 nmol/L, or about 80 nmol/L to about 100 nmol/L.

In certain embodiments, the concentration of small VLDL is decreased in a subject by administering an exendin, exendin agonist or GLP-1 receptor agonist. In certain embodiments, a decrease in the concentration of small VLDL may be a decrease of any amount. In various embodiments, the concentration of small VLDL is decreased by less than about 1 nmol/L, by about 1 nmol/L, about 1.5 nmol/L, about 2 nmol/L, about 2.5 nmol/L, about 3 nmol/L, about 3.5 nmol/L, or about 4 nmol/L.

In certain embodiments, the concentration of small VLDL is decreased in a subject by about 1 nmol/L to about 4 nmol/L, about 2 nmol/L to about 3 nmol/L, about 1 nmol/L to about 3 nmol/L, or about 2 nmol/L to about 4 nmol/L.

In certain embodiments, the concentration of total VLDL is decreased in a subject by administering an exendin, exendin agonist or GLP-1 receptor agonist. In certain embodiments, a decrease in the concentration of total VLDL may be a decrease of any amount. In various embodiments, the concentration of total VLDL is decreased by less than about 15 nmol/L, by about 15 nmol/L, about 13 nmol/L, about 12 nmol/L, about 11 nmol/L, about 10 nmol/L, about 9 nmol/L, about 8 nmol/L, about 7 nmol/L, about 6 nmol/L, or by less than about 6 nmol/L.

In various embodiments, the concentration of total VLDL is decreased by about 6 nmol/L to about 15 nmol/L, by about 15 nmol/L, about 13 nmol/L, about 12 nmol/L, about 11 nmol/L, about 10 nmol/L, about 9 nmol/L, about 8 nmol/L, about 7 mmol/L, about 6 nmol/L, or by less than about 6 nmol/L. In certain embodiments, the concentration of small LDL is decreased by about 120 nmol/L to about 300 nmol/L, the concentration of very small LDL is decreased by about 110 to about 240 nmol/L, and the concentration of total LDL is decreased by about 60 nmol/L to about 180 mmol/L. In certain embodiments, the concentration of small LDL is decreased by about 130 nmol/L to about 250 nmol/L, the concentration of very small LDL is decreased by about 115 to about 200 nmol/L, and the concentration of total LDL is decreased by about 50 nmol/L to about 150 nmol/L. In certain embodiments, the concentration of small LDL is decreased by about 140 nmol/L to about 200 nmol/L, the concentration of very small LDL is decreased by about 120 to about 180 nmol/L, and the concentration of total LDL is decreased by about 40 nmol/L to about 125 nmol/L.

The concentration of lipoprotein in a subject may be measured from any source available to the skilled artisan. By way of non-limiting example, the concentration of one or more lipoproteins may be measured in whole blood or plasma.

In certain embodiments, the concentration of one or more lipoproteins in a subject is measured in whole blood. In certain embodiments, the concentration of one or more lipoproteins in a subject is measured in whole plasma. In certain embodiments, the concentration of one or more lipoproteins in a subject is not measured in whole blood. In certain embodiments, the concentration of one or more lipoproteins in a subject is not measured in plasma.

In various embodiments of the present invention, the concentration of one or more lipoproteins is measured in whole blood or plasma, either or both of which may be fresh or frozen.

In the context of the present invention, lipoprotein concentrations are measured according to standard nuclear magnetic resonance techniques, where the measured amplitudes of characteristic lipid methyl group signals provide particle concentrations.

In certain embodiments, the present invention includes a method of providing an improved cardiovascular risk profile of a subject. In certain embodiments, the present invention includes a method of providing an improved cardiovascular risk profile of a subject in need thereof comprising administering to said subject a therapeutically effective amount of an exendin, exendin agonist or GLP-1 receptor agonist and measuring an increased concentration of large LDL, large HDL, total HDL or any combination of said lipoproteins, wherein the cardiovascular risk profile of said subject is improved.

In certain embodiments, the present invention includes a method of providing an improved cardiovascular risk profile of a subject who has a decreased level of large LDL, large HDL, total HDL or any combination thereof comprising administering to said subject a therapeutically effective amount of an exendin, exendin agonist or GLP-1 receptor agonist and measuring an increased concentration of large LDL, large HDL, total HDL or any combination of said lipoproteins, wherein the cardiovascular risk profile of said subject is improved.

In certain embodiments, the present invention includes a method of providing an improved cardiovascular risk profile of a subject who has an elevated level of small LDL, very small LDL, total LDL or any combination thereof comprising administering to said subject a therapeutically effective amount of an exendin, exendin agonist or GLP-1 receptor agonist and measuring a decreased concentration of small LDL, very small LDL, total LDL or any combination of said lipoproteins, wherein the cardiovascular risk profile of said subject is improved.

In certain embodiments, the present invention includes a method of providing an improved cardiovascular risk profile of a subject in need thereof comprising administering to said subject a therapeutically effective amount of an exendin, exendin agonist or GLP-1 receptor agonist and measuring an increased concentration of large LDL, large HDL, total HDL or any combination of said lipoproteins, and also measuring a decreased concentration of small LDL, very small LDL, total LDL or any combination of said lipoproteins.

In certain embodiments, providing an improved cardiovascular risk profile may include administering any exendin, exendin agonist or GLP-1 receptor agonist. In certain embodiments, providing an improved cardiovascular risk profile may include administering any exendin, exendin agonist or GLP-1 receptor agonist by any mode of administration known to the skilled artisan. Various exemplary, non-limiting exendins, exendin agonists and GLP-1 receptor agonists and modes of administration thereof are discussed herein.

In certain embodiments, an improved cardiovascular risk profile is a reduced risk of a cardiovascular disease or condition in a subject. In certain embodiments, an improved cardiovascular risk profile is a reduced risk of a cardiovascular disease or condition in a subject who has been previously identified as having one or more risk factors associated with a cardiovascular disease or condition. In certain embodiments, an improved cardiovascular risk profile is elimination of one or more risk factors associated with a cardiovascular disease or condition in a subject previously identified as having one or more risk factors associated with a cardiovascular disease or condition.

In certain embodiments, providing an improved cardiovascular risk profile is accomplished by measuring an increased concentration of large LDL, large HDL, total HDL or any combination of said lipoproteins. In certain embodiments, providing an improved cardiovascular risk profile is accomplished by measuring a decreased concentration of small LDL, very small LDL, total LDL or any combination of said lipoproteins. In certain embodiments, providing an improved cardiovascular risk profile is accomplished by measuring an increased concentration of large LDL, large HDL, total HDL or any combination of said lipoproteins and measuring a decreased concentration of small LDL, very small LDL, total LDL or any combination of said lipoproteins.

Also provided herein are methods of treating a subject with an elevated level of small LDL, very small LDL or total LDL or any combination of said lipoproteins, comprising administering to said subject a therapeutically effective amount of an exendin, exendin agonist or GLP-1 receptor agonist, wherein the concentration of small LDL, very small LDL, total LDL or any combination of said lipoproteins is decreased in said subject.

In certain embodiments, there are provided methods for treating a subject with a decreased level of large LDL, large HDL, total HDL or any combination of said lipoproteins, comprising administering to said subject a therapeutically effective amount of an exendin, exendin agonist or GLP-1 receptor agonist, wherein the concentration of large LDL, large HDL, total HDL or any combination of said lipoproteins is increased in said subject.

In certain embodiments, prophylactic treatment is provided. In certain embodiments, prophylactic treatment may include preventing a disease or condition. In certain embodiments, prophylactic treatment prevents worsening of a disease or condition. For example, in certain embodiments, prophylactic treatment prevents worsening of an elevated small LDL, very small LDL, or total LDL level. In certain embodiments, prophylactic treatment maintains a small LDL, very small LDL, or total LDL levels that are not elevated as not elevated.

In certain embodiments, therapeutic treatment is provided herein. Therapeutic treatment may result, for example without limitation, in ameliorating symptoms of any disease or condition, in reducing a concentration of one or more elevated levels, such as for example small LDL, very small LDL, or total LDL level, in increasing a concentration of one or more decreased level, such as for example large LDL, large HDL, total HDL, or in eliminating a disease or condition in a subject.

In certain embodiments, treatment on an acute or chronic basis is contemplated. In addition, treatment on an acute basis may be extended to chronic treatment, if so indicated. In certain embodiments, treatment of a subject may include administering any exendin, exendin agonist or GLP-1 receptor agonist by any mode of administration known to the skilled artisan. Various exemplary, non-limiting exendins, exendin agonist and GLP-1 receptor agonists, and modes of administration thereof are discussed herein.

In certain embodiments, the present invention includes a method of increasing the average particle size of LDL or HDL or both in a subject in need thereof comprising administering to said subject a therapeutically effective amount of an exendin, exendin agonist or GLP-1 receptor agonist, wherein the particle size of LDL or HDL or both is increased in said subject.

In certain embodiments, the present invention includes a method of increasing the average particle size of LDL in a subject in need thereof comprising administering to said subject a therapeutically effective amount of an exendin, exendin agonist or GLP-1 receptor agonist, wherein the particle size of LDL is increased in said subject.

In certain embodiments, the present invention includes a method of increasing the average particle size of HDL in a subject in need thereof comprising administering to said subject a therapeutically effective amount of an exendin, exendin agonist or GLP-1 receptor agonist, wherein the particle size of HDL is increased in said subject.

In certain embodiments, the present invention includes a method of increasing the average particle size of LDL and HDL in a subject in need thereof comprising administering to said subject a therapeutically effective amount of an exendin, exendin agonist or GLP-1 receptor agonist, wherein the particle sizes of LDL and HDL are increased in said subject.

In certain embodiments, methods of increasing average lipoprotein particle size may include administering any exendin, exendin agonist or GLP-1 receptor agonist. In certain embodiments, methods of increasing average lipoprotein particle size may include administering any exendin, exendin agonist or GLP-1 receptor agonist by any mode of administration known to the skilled artisan. Various exemplary, non-limiting exendins, exendin agonist and GLP-1 receptor agonists, and modes of administration thereof are discussed herein.

In certain embodiments, the particle size of LDL or HDL or both is increased and the concentrations of one or more lipoproteins are altered. In certain embodiments, the particle size of LDL or HDL or both is increased and the concentration of very small LDL, small LDL, total LDL, small VLDL, total VLDL or any combination of such lipoproteins is decreased. In certain embodiments, the particle size of LDL or HDL or both is increased and the concentration of small LDL, very small LDL, total LDL or any combination of such lipoproteins is decreased.

In certain embodiments, the particle size of LDL or HDL or both is increased and the concentration of large LDL, large HDL, total HDL or any combination of such lipoproteins is increased. In certain embodiments, the particle size of LDL or HDL or both is increased and the concentration of small LDL, very small LDL, total LDL or any combination of such lipoproteins is decreased and the concentration of large LDL, large HDL, total HDL or any combination of such lipoproteins is increased.

Contemplated embodiments further include a method of increasing the average particle size of LDL or HDL or both in a subject who has decreased level of large LDL, large HDL, total HDL or any combination thereof comprising administering to said subject a therapeutically effective amount of an exendin, exendin agonist or GLP-1 receptor agonist, wherein the average particle size of LDL or HDL is increased in said subject. In certain embodiments, there are provided methods of increasing the average particle size of LDL or HDL or both in a subject who has an elevated level of small LDL, very small LDL, total LDL or any combination thereof comprising administering to said subject a therapeutically effective amount of exendin, exendin agonist or GLP-1 receptor agonist, wherein the average particle size of LDL or HDL is increased in said subject.

In the context of the present invention, an increase in average particle size refers to an increase in the diameter of the particle. In certain embodiments, an increase in average particle size may be an increase of any amount.

In certain embodiments, the average particle size of LDL is increased by less than about 0.1 nm, by about 0.1 nm, about 0.2 nm, about 0.25 nm, about 0.3 nm, about 0.35 nm, about 0.4 nm, about 0.45 nm, about 0.5 nm, about 0.6 nm, about 0.7 nm, about 1 nm, about 1.5 nm, about 2 nm, about 2.5 nm, about 3 nm, about 4 nm, about 5 nm or by more than about 5 nm. In other embodiments, the average particle size of LDL is increased by about 0.1 nm to about 5 nm, about 0.1 to about 2.5 m-n, about 0.1 nm to about 0.7 nm, by about 0.2 nm to about 0.6 nm, by about 0.3 nm to about 0.5 nm, by about 0.3 nm to about 0.4 nm, or about 0.2 nm to about 0.4 nm.

In certain embodiments, the average particle size of HDL is increased by less than about 0.05 nm, by about 0.05 nm, about 0.07 nm, about 0.08 nm, about 0.09 nm, about 0.1 nm, about 0.11, about 0.12 nm, about 0.13 nm, about 0.14 nm, about 0.15 nm, about 0.16 nm, about 0.17 nm, about 0.18 nm, about 0.19 nm, about 0.2 nm, about 0.25 nm, about 0.3 nm, about 0.35 nm, about 0.4 nm, about 0.45 nm, about 0.5 nm, or by more that about 0.5 nm.

In certain embodiments, both LDL and HDL average particle size are increased by about 0.05 nm to about 6 nm. In certain embodiments, both LDL and HDL average particle sizes are increased by about 0.1 nm to about 0.4 nm. In certain embodiments, both LDL and HDL average particle sizes are increased by about 0.11 nm to about 0.33 nm.

In the context of the present invention, lipoprotein subclass particle concentrations and average particle diameters are measured using proton nuclear magnetic resonance spectroscopy using the algorithm of LipoScience, Inc. See e.g., Festa et al., Nuclear Magnetic Resonance Lipoprotein Abnormalities in Prediabetic Subjects in the Insulin Resistance Artherosclerosis Study, Circulation, 111:3465-3472 (2005).

In certain embodiments, a subject may include any animal. In certain embodiments, a subject may include any mammal. In exemplary embodiments, a subject is a human. In various other embodiments, a subject may include pets (e.g., dogs and cats and the like), as well as livestock, such as for example horses, cows, pigs, sheep, and the like.

In certain embodiments, the methods of the present invention comprise the identification of a subject in need of administration of one or more exendins, exendin agonists or GLP-1 receptor agonists. In certain embodiments, a subject in need is any subject who would benefit from the administration of one or more exendins, exendin agonists or GLP-1 receptor agonists. Any effective criteria may be used to determine that a subject may benefit from administration of one or more exendins, exendin agonists or GLP-1 receptor agonists. Procedures for determining a subject who is in need may include, for example, clinical tests, laboratory tests, physical examination, personal interviews and assessment of family history.

In certain embodiments, a subject in need is any subject who would benefit from a decrease in the concentration of small LDL, very small LDL, total LDL or any combination of said lipoproteins or an increase in the concentration of large LDL, large HDL, total HDL or any combination of said lipoproteins. Any effective criteria may be used to determine that a subject may benefit from administration of one or more exendins, exendin agonists or GLP-1 receptor agonists to decrease the concentration of small LDL, very small LDL, total LDL or any combination of said lipoproteins or to increase the concentration of large LDL, large HDL, total HDL or any combination of said lipoproteins. For example, methods are known to the skilled artisan for detecting elevated levels of small LDL, very small LDL, total LDL or any combination of said lipoproteins and for detecting decreased levels of large LDL, large HDL, total HDL or any combination of said lipoproteins. Also for example, methods for detecting conditions associated with elevated levels of small LDL, very small LDL, total LDL or any combination of said lipoproteins or decreased levels of large LDL, large HDL, or total HDL or any combination of said lipoproteins, such as for example cardiovascular diseases or conditions, are known to those of skill in the art.

In certain embodiments, a subject in need is an individual who appears healthy. In certain embodiments, an individual who appears healthy is an individual who has not been diagnosed with any disease or condition selected from the group consisting of diabetes, prediabetes, and one or more cardiovascular diseases or conditions. In certain embodiments, an individual who appears healthy is an individual who does not have any disease or condition selected from the group consisting of diabetes, prediabetes, and one or more cardiovascular diseases or conditions.

In certain embodiments, an individual who appears healthy has not been diagnosed with elevated levels of small LDL, very small LDL, total LDL or any combination of said lipoproteins, or decreased levels of large LDL or large HDL, total HDL or any combination of said lipoproteins. In certain embodiments, an individual who appears healthy does not have a elevated levels of small LDL, very small LDL, total LDL or any combination of said lipoproteins, or decreased levels of large LDL or large HDL, total HDL or any combination of said lipoproteins.

In certain embodiments, a subject in need has one or more cardiovascular diseases or conditions. In certain embodiments, a subject in need has been diagnosed with one or more cardiovascular diseases or conditions. Cardiovascular diseases or conditions may include by way of non-limiting example angina, arrhythmia, atrial fibrillation, high blood pressure, high cholesterol, myocardial infarction, heart failure, arteriosclerosis, atherosclerosis, angina, stroke, pericarditis, coronary artery disease, hypertrophic cardiomyopathy, and hyperlipidemia. In certain embodiments, cardiovascular diseases or conditions may include without limitation angina, arrhythmia, atrial fibrillation, high blood pressure, high cholesterol, heart failure, arteriosclerosis, atherosclerosis, pericarditis, coronary artery disease, hypertrophic cardiomyopathy, and hyperlipidemia. Additional cardiovascular diseases and conditions are apparent to those of skill in the art.

In certain embodiments, a subject in need is at risk for one or more cardiovascular diseases or conditions. Risk factors for cardiovascular diseases or conditions include any risk factor known by one skilled in the art. Non-limiting examples of risk factors for cardiovascular diseases or conditions may include age, diabetes, tobacco smoking, elevated cholesterol levels, high levels of LDL, high fibrinogen and PAI-1 blood concentration, high blood pressure, elevated homocysteine, elevated blood levels of asymmetric dimethylarginine, being overweight, being obese, genetic factors, family history of cardiovascular disease, physical inactivity, being male.

In certain embodiments, a subject in need is overweight or obese. While in an embodiment, obesity is generally defined as a body mass index over 30, in another embodiment, any subject, who needs or wishes to reduce body weight is included in the scope of obese. Body mass index (BMI) can be measured by methods well known in the art. In certain embodiments, a subject in need is less then 20 years old, 20 years or older, 25 years or older, 35 years or older, 45 years or older, 55 years or older, 65 years or older, 75 years or older, 85 years or older, or 95 years or older. In certain embodiments, the subject is overweight and is 45 years of age or older.

In certain embodiments, a subject in need has prediabetes. In certain embodiments, a subject in need has been diagnosed with prediabetes.

In certain embodiments, a subject in need has diabetes. In certain embodiments, a subject in need has been diagnosed with diabetes. In certain embodiments, a subject in need is at risk for diabetes. Exemplary non-limiting risk factors for diabetes include being overweight, having hypertension, having high cholesterol, being African American, being Mexican American, being Native American, having a family history of diabetes, having genetic factors, lacking physical inactivity, having impaired glucose tolerance, or having a previous history of gestational diabetes.

In certain embodiments, a subject in need has type 1 diabetes, type 2 diabetes or gestational diabetes. In certain embodiments, a subject in need has been diagnosed with type 1 diabetes, type 2 diabetes or gestational diabetes. In certain embodiments, a subject in need is at risk for type 1 diabetes, type 2 diabetes or gestational diabetes.

In certain embodiments, a subject in need has diabetes and one or more cardiovascular diseases or conditions. In certain embodiments, a subject in need has been diagnosed with diabetes and one or more cardiovascular diseases or conditions. In certain embodiments, a subject in need is at risk for diabetes and one or more cardiovascular diseases or conditions.

In certain embodiments, a subject in need has prediabetes and one or more cardiovascular diseases or conditions. In certain embodiments, a subject in need has been diagnosed with prediabetes and one or more cardiovascular diseases or conditions.

In certain embodiments, a subject in need does not have prediabetes. In certain embodiments, a subject in need does not have diabetes. In certain embodiments, a subject in need does not have prediabetes or diabetes. In certain embodiments, a subject in need does not have one or more cardiovascular diseases or conditions. In certain embodiments, a subject in need does not have one or more cardiovascular diseases or conditions and diabetes. In certain embodiments, a subject in need does not have one or more cardiovascular diseases or conditions and prediabetes. In certain embodiments, a subject in need is not in need of glucose lowering. In certain embodiments, a subject in need is not hyperglycemic. In certain embodiments, a subject in need is normoglycemic. In certain embodiments, a subject in need is hypoglycemic. Regarding a subject who has an decreased large LDL, large HDL, total HDL level, or any combination thereof, or who has an elevated level of small LDL, very small LDL, total LDL or any combination thereof, in certain embodiments such a subject is not in need of glucose lowering; in certain embodiments the subject is not hyperglycemic; in certain embodiments, the subject is normoglycemic; in certain embodiments, the subject is hypoglycemic.

In certain embodiments, a subject in need has not been diagnosed with prediabetes. In certain embodiments, a subject in need has not been diagnosed with diabetes. In certain embodiments, a subject in need has not been diagnosed with one or more cardiovascular diseases or conditions. In certain embodiments, a subject in need has not been diagnosed with one or more cardiovascular diseases or conditions and diabetes. In certain embodiments, a subject in need has not been diagnosed with prediabetes and one or more cardiovascular diseases or conditions.

In certain embodiments, a subject in need has a family history of diabetes. In certain embodiments, a subject in need has a family history of one or more cardiovascular diseases or conditions. In certain embodiments, a subject in need has a family history of diabetes and one or more cardiovascular diseases or conditions. As used herein, a family history means more than one, more than two, more than three, or more than four generations of a family have been diagnosed with a certain disease or condition.

In certain embodiments, a subject in need does not have a family history of diabetes. In certain embodiments, a subject in need does not have a family history of one or more cardiovascular diseases or conditions. In certain embodiments, a subject in need does not have a family history of diabetes and one or more cardiovascular diseases or conditions.

In certain embodiments, a subject in need is a subject who has elevated levels of small LDL, very small LDL, total LDL or any combination of said lipoproteins, or decreased levels of large LDL, large HDL, total HDL or any combination of said lipoproteins. In certain embodiments, a subject in need has been diagnosed with elevated levels of small LDL, very small LDL, total LDL or any combination of said lipoproteins, or decreased levels of large LDL, large HDL, total HDL or any combination of said lipoproteins. In certain embodiments, a subject in need is at risk for elevated levels of small LDL, very small LDL, total LDL or any combination of said lipoproteins, or decreased levels of large LDL, large HDL, total HDL or any combination of said lipoproteins.

Exemplary, non-limiting subjects at risk for elevated levels of small LDL, very small LDL, total LDL or any combination of said lipoproteins, or decreased levels of large LDL, large HDL, total HDL or any combination of said lipoproteins include subjects with prediabetes, diabetes, one or more cardiovascular diseases or conditions, or a family history of any thereof.

In certain embodiments, a subject with an elevated level of small LDL, very small LDL, total LDL or any combination of said lipoproteins, or decreased level of large LDL, large HDL, total HDL or any combination of said lipoproteins includes a subject who has (diagnosed or undiagnosed) an elevated concentration of small LDL, very small LDL, total LDL or any combination of said lipoproteins, or a decreased concentration of large LDL, large HDL, total HDL or any combination of said lipoproteins or a subject who has been diagnosed with an elevated concentration of small LDL, very small LDL, total LDL or any combination of said lipoproteins, or a decreased concentration of large LDL, large HDL, total HDL or any combination of said lipoproteins.

In certain embodiments, a subject with elevated levels of small LDL, very small LDL, total LDL or any combination of said lipoproteins has a concentration of small LDL, very small LDL, total LDL or any combination of said lipoproteins that is higher than normal for that subject when healthy. In another aspect, elevated levels of small LDL, very small LDL, total LDL or any combination of said lipoproteins is a concentration in a subject that is higher than normal for a healthy population of the same species as the subject.

In various embodiments, elevated levels of small LDL, very small LDL, total LDL or any combination of said lipoproteins are concentrations that are about 10%, about 25%, about 50%, about 75%, about 100%, about 200%, about 300%, about 500%, about 1000%, or more than about 1000% higher than normal for the subject when healthy or for a healthy population of the same species as the subject.

In various embodiments, an elevated level of total LDL is a total LDL level that is more than about 1150 nmol/L, more than about 1200 nmol/L, more than about 1300 nmol/L, more than about 1500 nmol/L, more than about 2000 nmol/L, or more than about 2500 nmol/L.

In other embodiments, a subject with a decreased level of large LDL, large HDL, total HDL or any combination of said lipoproteins has a concentration of large LDL, large HDL, total HDL or any combination of said lipoproteins that is lower than normal for that subject when healthy. In another aspect, a decreased level of large LDL, large HDL, total HDL or any combination of said lipoproteins is a concentration in a subject that is lower than normal for a healthy population of the same species as the subject.

In various embodiments, a decreased level of large LDL, large HDL, total HDL or any combination of said lipoproteins include concentrations that are about 10%, about 25%, about 50%, about 75%, about 80%, about 85%, about 90%, about 95%, about 98%, or even about 99% less than normal for the subject when healthy or for a healthy population of the same species as the subject.

In other embodiments, a decreased level of total HDL is a total HDL level that is less than about 31 μmol/L, about 30 μmol/L, about 27.5 μmol/L, about 25 μmol/L, about 20 μmol/L, about 15 μmol/L, or less than about 15 μmol/L.

In certain embodiments, a subject with or at risk for elevated levels of small LDL, very small LDL, total LDL or any combination of said lipoproteins, or decreased levels of large LDL, large HDL, total HDL or any combination of said lipoproteins has one or more cardiovascular diseases or conditions. In certain embodiments, a subject with or at risk for elevated levels of small LDL, very small LDL, total LDL or any combination of said lipoproteins, or decreased levels of large LDL, large HDL, total HDL or any combination of said lipoproteins has been diagnosed with one or more cardiovascular diseases or conditions. In certain embodiments, a subject with or at risk for elevated levels of small LDL, very small LDL, total LDL or any combination of said lipoproteins, or decreased levels of large LDL, large HDL, total HDL or any combination of said lipoproteins is at risk for one or more cardiovascular diseases or conditions.

In certain embodiments, a subject with or at risk for elevated levels of small LDL, very small LDL, total LDL or any combination of said lipoproteins, or decreased levels of large LDL, large HDL, total HDL or any combination of said lipoproteins has prediabetes. In certain embodiments, a subject with or at risk for elevated levels of small LDL, very small LDL, total LDL or any combination of said lipoproteins, or decreased levels of large LDL, large HDL, total HDL or any combination of said lipoproteins has been diagnosed with prediabetes.

In certain embodiments, a subject with or at risk for elevated levels of small LDL, very small LDL, total LDL or any combination of said lipoproteins, or decreased levels of large LDL, large HDL, total HDL or any combination of said lipoproteins has diabetes. In certain embodiments, a subject with or at risk for elevated levels of small LDL, very small LDL, total LDL or any combination of said lipoproteins, or decreased levels of large LDL, large HDL, total HDL or any combination of said lipoproteins has been diagnosed with diabetes. In certain embodiments, a subject with or at risk for elevated levels of small LDL, very small LDL, total LDL or any combination of said lipoproteins, or decreased levels of large LDL, large HDL, total HDL or any combination of said lipoproteins is at risk for diabetes.

In certain embodiments, a subject with or at risk for elevated levels of small LDL, very small LDL, total LDL or any combination of said lipoproteins, or decreased levels of large LDL, large HDL, total HDL or any combination of said lipoproteins has type 1 diabetes, type 2 diabetes or gestational diabetes. In certain embodiments, a subject with or at risk for elevated levels of small LDL, very small LDL, total LDL or any combination of said lipoproteins, or decreased levels of large LDL, large HDL, total HDL or any combination of said lipoproteins has been diagnosed with type 1 diabetes, type 2 diabetes or gestational diabetes. In certain embodiments, a subject with or at risk for elevated levels of small LDL, very small LDL, total LDL or any combination of said lipoproteins, or decreased levels of large LDL, large HDL, total HDL or any combination of said lipoproteins is at risk for type 1 diabetes, type 2 diabetes or gestational diabetes.

In certain embodiments, a subject with or at risk for elevated levels of small LDL, very small LDL, total LDL or any combination of said lipoproteins, or decreased levels of large LDL, large HDL, total HDL or any combination of said lipoproteins has diabetes and one or more cardiovascular diseases or conditions. In certain embodiments, a subject with or at risk for elevated levels of small LDL, very small LDL, total LDL or any combination of said lipoproteins, or decreased levels of large LDL, large HDL, total HDL or any combination of said lipoproteins has been diagnosed with diabetes and one or more cardiovascular diseases or conditions. In certain embodiments, a subject with or at risk for elevated levels of small LDL, very small LDL, total LDL or any combination of said lipoproteins, or decreased levels of large LDL, large HDL, total HDL or any combination of said lipoproteins is at risk for diabetes and one or more cardiovascular diseases or conditions.

In certain embodiments, a subject with or at risk for elevated levels of small LDL, very small LDL, total LDL or any combination of said lipoproteins, or decreased levels of large LDL, large HDL, total HDL or any combination of said lipoproteins has prediabetes and one or more cardiovascular diseases or conditions. In certain embodiments, a subject with or at risk for elevated levels of small LDL, very small LDL, total LDL or any combination of said lipoproteins, or decreased levels of large LDL, large HDL, total HDL or any combination of said lipoproteins has been diagnosed with prediabetes and one or more cardiovascular diseases or conditions.

In certain embodiments, a subject with or at risk for elevated levels of small LDL, very small LDL, total LDL or any combination of said lipoproteins, or decreased levels of large LDL, large HDL, total HDL or any combination of said lipoproteins does not have prediabetes. In certain embodiments, a subject with or at risk for elevated levels of small LDL, very small LDL, total LDL or any combination of said lipoproteins, or decreased levels of large LDL, large HDL, total HDL or any combination of said lipoproteins does not have diabetes. In certain embodiments, a subject with or at risk elevated levels of small LDL, very small LDL, total LDL or any combination of said lipoproteins, or decreased levels of large LDL, large HDL, total HDL or any combination of said lipoproteins does not have one or more cardiovascular diseases or conditions.

In certain embodiments, a subject with or at risk for elevated levels of small LDL, very small LDL, total LDL or any combination of said lipoproteins, or decreased levels of large LDL, large HDL, total HDL or any combination of said lipoproteins does not have one or more cardiovascular diseases or conditions and prediabetes. In certain embodiments, a subject with or at risk for elevated levels of small LDL, very small LDL, total LDL or any combination of said lipoproteins, or decreased levels of large LDL, large HDL, total HDL or any combination of said lipoproteins does not have one or more cardiovascular diseases or conditions and diabetes.

In certain embodiments, a subject with or at risk for elevated levels of small LDL, very small LDL, total LDL or any combination of said lipoproteins, or decreased levels of large LDL, large HDL, total HDL or any combination of said lipoproteins has not been diagnosed with prediabetes. In certain embodiments, a subject with or at risk for elevated levels of small LDL, very small LDL, total LDL or any combination of said lipoproteins, or decreased levels of large LDL, large HDL, total HDL or any combination of said lipoproteins has not been diagnosed with diabetes. In certain embodiments, a subject with or at risk for elevated levels of small LDL, very small LDL, total LDL or any combination of said lipoproteins, or decreased levels of large LDL, large HDL, total HDL or any combination of said lipoproteins has not been diagnosed with one or more cardiovascular diseases or conditions.

In certain embodiments, a subject with or at risk for elevated levels of small LDL, very small LDL, total LDL or any combination of said lipoproteins, or decreased levels of large LDL, large HDL, total HDL or any combination of said lipoproteins has not been diagnosed with one or more cardiovascular diseases or conditions and prediabetes. In certain embodiments, a subject with or at risk for elevated levels of small LDL, very small LDL, total LDL or any combination of said lipoproteins, or decreased levels of large LDL, large HDL, total HDL or any combination of said lipoproteins has not been diagnosed with one or more cardiovascular diseases or conditions and diabetes.

In certain embodiments, a subject with or at risk for elevated levels of small LDL, very small LDL, total LDL or any combination of said lipoproteins, or decreased levels of large LDL, large HDL, total HDL or any combination of said lipoproteins has a family history of diabetes. In certain embodiments, a subject with or at risk for elevated levels of small LDL, very small LDL, total LDL or any combination of said lipoproteins, or decreased levels of large LDL, large HDL, total HDL or any combination of said lipoproteins has a family history of one or more cardiovascular diseases or conditions. In certain embodiments, a subject with or at risk for elevated levels of small LDL, very small LDL, total LDL or any combination of said lipoproteins, or decreased levels of large LDL, large HDL, total HDL or any combination of said lipoproteins has a family history of diabetes and one or more cardiovascular diseases or conditions.

In certain embodiments, a subject with or at risk for elevated levels of small LDL, very small LDL, total LDL or any combination of said lipoproteins, or decreased levels of large LDL, large HDL, total HDL or any combination of said lipoproteins does not have a family history of diabetes. In certain embodiments, a subject with or at risk for elevated levels of small LDL, very small LDL, total LDL or any combination of said lipoproteins, or decreased levels of large LDL, large HDL, total HDL or any combination of said lipoproteins does not have a family history of one or more cardiovascular diseases or conditions. In certain embodiments, a subject with or at risk for elevated levels of small LDL, very small LDL, total LDL or any combination of said lipoproteins, or decreased levels of large LDL, large HDL, total HDL or any combination of said lipoproteins does not have a family history of diabetes and one or more cardiovascular diseases or conditions.

In certain embodiments, a subject with or at risk for elevated levels of small LDL, very small LDL, total LDL or any combination of said lipoproteins, or decreased levels of large LDL, large HDL, total HDL or any combination of said lipoproteins is overweight.

In further embodiments, a subject with or at risk for elevated levels of small LDL, very small LDL, total LDL or any combination of said lipoproteins, or decreased levels of large LDL, large HDL, total HDL or any combination of said lipoproteins is less then 20 years old, 20 years or older, 25 years or older, 35 years or older, 45 years or older, 55 years or older, 65 years or older, 75 years or older, 85 years or older, or 95 years or older.

In certain embodiments, a subject in need does not have elevated levels of small LDL, very small LDL, total LDL or any combination of said lipoproteins, or decreased levels of large LDL, large HDL, total HDL or any combination of said lipoproteins. In certain embodiments, a subject in need does not have elevated levels of small LDL, very small LDL, or total LDL, if the subject has a concentration of small LDL, very small LDL or total LDL lower than a subject with an elevated level of small LDL, very small LDL, or total LDL as defined herein. In certain embodiments, a subject in need does not have decreased levels of large LDL, large HDL, or total HDL if the subject has a concentration of large LDL, large HDL, or total HDL higher than a subject with a decreased level of large LDL, large HDL, or total HDL as defined herein.

### Exendin and Exendin Agonist Compounds

In some embodiments of the methods provided herein, any exendin or exendin agonist may be administered. Exemplary non-limiting exendins and exendin agonists include or comprise exendin-3, exendin-4, and C-terminally truncated exendin peptides of 25, 26, 27, 28, 29, 30, 31, 32, 33, 34, 35, 36, 37, or 38 amino acids in length.

In certain embodiments, exendin agonists include or comprise without limitation exendin-4 acid, exendin-4 (1-30), exendin-4 (1-30) amide, exendin-4(1-27), exendin-4(1-28), exendin-4 (1-28)amide, ¹⁴Leu, ²⁵Phe exendin-4, ¹⁴Leu exendin-4, ¹⁴Leu exendin-4(1-28) amide, ¹⁴Leu exendin-4(1-28), and ¹⁴Leu, ²⁵Phe exendin-4 (1-28).

Exendin agonist compounds contemplated herein include or comprise without limitation those described in Published Application WO99/07404, filed Aug. 6, 1998, entitled, “Novel Exendin Agonist Compounds,” which claims the benefit of U.S. Provisional Application No. 60/055,404, filed Aug. 8, 1997, and corresponding U.S. Pat. No. 6,956,026 issued Oct. 18, 2005, each of which are herein incorporated by reference in their entireties and for all purposes, which purposes include the exendin and exendin agonist compounds and formulations thereof taught therein. Included, for example without limitation, are compounds comprising a structure of Formula I (SEQ ID NO:_):

I
Xaa¹ Xaa² Xaa³ Gly Thr Xaa⁴ Xaa⁵ Xaa⁶ Xaa⁷ Xaa⁸
Ser Lys Gln Xaa⁹ Glu Glu Glu Ala Val Arg Leu
Xaa¹⁰ Xaa¹¹ Xaa¹² Xaa¹³ Leu Lys Asn Gly Gly Xaa¹⁴
Ser Ser Gly Ala Xaa¹⁵ Xaa¹⁶ Xaa¹⁷ Xaa¹⁸

wherein Xaa¹ is H is, Arg or Tyr; Xaa² is Ser, Gly, Ala or Thr; Xaa³ is Ala, Asp or Glu; Xaa⁴ is Phe, Tyr or naphthylalanine; Xaa⁵ is Thr or Ser; Xaa⁶ is Ser or Thr; Xaa⁷ is Asp or Glu; Xaa⁸ is Leu, Ile, Val, pentylglycine or Met; Xaa⁹ is Leu, Ile, pentylglycine, Val or Met; Xaa¹⁰ is Phe, Tyr or naphthylalanine; Xaa¹¹ is Ile, Val, Leu, pentylglycine, tert-butylglycine or Met; Xaa¹² is Glu or Asp; Xaa¹³ is Trp, Phe, Tyr, or naphthylalanine; Xaa¹⁴, Xaa¹⁵, Xaa¹⁶ and Xaa¹⁷ are independently Pro, homoproline, 3Hyp, 4Hyp, thioproline, N-alkylglycine, N-alkylpentylglycine or N-alkylalanine; Xaa¹⁸ is Ser, Thr, Tyr, or C-terminal free acid or amidated derivatives thereof. The term “C-terminal free acid” and like terms in the context of amino acids refers to the presence of a C-terminal carboxylate moiety. The term “C-terminal amidated derivative” and like terms in the context of amino acids refers to the presence of a C-terminal capping amide (e.g., —NH₂, —NHR¹, —NR¹R², wherein R¹ and R² are independently alkyl, and the like).

In certain embodiments contemplating compounds with structure of Formula I, N-alkyl groups for N-alkylglycine, N-alkylpentylglycine and N-alkylalanine include lower alkyl groups of 1 to about 6 carbon atoms, or in other embodiments 1 to 4 carbon atoms. In certain embodiments, exendin agonist compounds include those wherein Xaa¹ is H is or Tyr. In certain embodiments, Xaa¹ is His. In certain embodiments, compounds are included wherein Xaa² is Gly. In certain embodiments, compounds are contemplated wherein Xaa⁹ is Leu, pentylglycine or Met. In certain embodiments, exemplary compounds include without limitation those wherein Xaa¹³ is Trp or Phe.

In certain embodiments contemplating compounds with structure of Formula I, exemplary compounds are those in which Xaa⁴ is Phe or naphthylalanine, Xaa¹¹ is Ile or Val, and Xaa¹⁴, Xaa¹⁵, Xaa¹⁶ and Xaa¹⁷ are independently selected from the group consisting of Pro, homoproline, thioproline and N-alkylalanine. In certain embodiments, N-alkylalanine has a N-alkyl group of 1 to about 6 carbon atoms. In certain embodiments, Xaa¹⁵, Xaa¹⁶ and Xaa¹⁷ are the same amino acid residue. In certain embodiments, Xaa¹⁸ is Ser or Tyr.

In certain embodiments, compounds are those of Formula I wherein Xaa¹ is H is or Tyr; Xaa² is Gly; Xaa⁴ is Phe or naphthylalanine; Xaa⁹ is Leu, pentylglycine or Met; Xaa¹⁰ is Phe or naphthylalanine; Xaa¹¹ is Ile or Val; Xaa¹⁴, Xaa¹⁵, Xaa¹⁶ and Xaa¹⁷ are independently selected from Pro, homoproline, thioproline or N-alkylalanine; Xaa¹⁸ is Ser or Tyr, wherein Xaa¹⁸ is amidated.

In certain embodiments, compounds include those of Formula I wherein: Xaa¹ is H is or Arg; Xaa² is Gly; Xaa³ is Ala, Asp or Glu; Xaa⁴ is Phe or napthylalanine; Xaa⁵ is Thr or Ser; Xaa⁶ is Ser or Thr; Xaa⁷ is Asp or Glu; Xaa⁸ is Leu or pentylglycine; Xaa⁹ is Leu or pentylglycine; Xaa¹⁰ is Phe or naphthylalanine; Xaa¹¹ is Ile, Val or t-butylglycine; Xaa¹² is Glu or Asp; Xaa¹³ is Trp or Phe; Xaa¹⁴, Xaa¹⁵, Xaa¹⁶, and Xaa¹⁷ are independently Pro, homoproline, thioproline, or N-methylalanine; Xaa¹⁸ is C-terminal free acid or amidated Ser or Tyr.

In certain embodiments contemplating compounds with structure of Formula I, compounds are contemplated wherein Xaa⁹ is Leu, Ile, Val or pentylglycine, and Xaa¹³ is Phe, Tyr or naphthylalanine.

In certain embodiments, exendin agonist compounds comprise or include those described in Published Application WO99/25727, filed Nov. 13, 1998, entitled, “Novel Exendin Agonist Compounds,” which claims the benefit of U.S. Provisional Application No. 60/065,442, filed Nov. 14, 1997, and corresponding U.S. Pat. No. 7,223,725 issued May 29, 2007, each of which are herein incorporated by reference in their entireties and for all purposes, which purposes include the exendin and exendin agonist compounds and formulations thereof taught therein. Included without limitation are compounds comprising the structure of the Formula II (SEQ ID NO:_):

II
Xaa¹ Xaa² Xaa³ Gly Xaa⁵ Xaa⁶ Xaa⁷ Xaa⁸ Xaa⁹ Xaa¹⁰
Xaa¹¹ Xaa¹² Xaa¹³ Xaa¹⁴ Xaa¹⁵ Xaa¹⁶ Xaa¹⁷ Ala
Xaa¹⁹ Xaa²⁰ Xaa²¹ Xaa²² Xaa²³ Xaa²⁴ Xaa²⁵ Xaa²⁶
Xaa²⁷ Xaa²⁸-Z₁;

wherein Xaa¹ is H is, Arg or Tyr; Xaa² is Ser, Gly, Ala or Thr; Xaa³ is Ala, Asp or Glu; Xaa⁵ is Ala or Thr; Xaa⁶ is Ala, Phe, Tyr or naphthylalanine; Xaa⁷ is Thr or Ser; Xaa⁸ is Ala, Ser or Thr; Xaa⁹ is Asp or Glu; Xaa¹⁰ is Ala, Leu, Ile, Val, pentylglycine or Met; Xaa¹¹ is Ala or Ser; Xaa¹² is Ala or Lys; Xaa¹³ is Ala or Gln; Xaa¹⁴ is Ala, Leu, Ile, pentylglycine, Val or Met; Xaa¹⁵ is Ala or Glu; Xaa¹⁶ is Ala or Glu; Xaa¹⁷ is Ala or Glu; Xaa¹⁹ is Ala or Val; Xaa²⁰ is Ala or Arg; Xaa²¹ is Ala or Leu; Xaa²² is Ala, Phe, Tyr or naphthylalanine; Xaa²³ is Ile, Val, Leu, pentylglycine, tert-butylglycine or Met; Xaa²⁴ is Ala, Glu or Asp; Xaa²⁵ is Ala, Trp, Phe, Tyr or naphthylalanine; Xaa²⁶ is Ala or Leu; Xaa²⁷ is Ala or Lys; Xaa²⁸ is Ala or Asn; Z₁ is absent or —OH, —NH₂, Gly-Z₂, Gly Gly-Z₂, Gly Gly Xaa³¹-Z₂, Gly Gly Xaa³¹ Ser-Z₂, Gly Gly Xaa³¹ Ser Ser-Z₂, Gly Gly Xaa³¹ Ser Ser Gly-Z₂, Gly Gly Xaa³¹ Ser Ser Gly Ala-Z₂, Gly Gly Xaa³¹ Ser Ser Gly Ala Xaa³⁶-Z₂, Gly Gly Xaa³¹ Ser Ser Gly Ala Xaa³⁶ Xaa³⁷-Z₂ or Gly Gly Xaa³¹ Ser Ser Gly Ala Xaa³⁶ Xaa³⁷ Xaa³⁸-Z₂, wherein Xaa³¹, Xaa³⁶, Xaa³⁷ and Xaa³⁸ are independently Pro, homoproline, 3Hyp, 4Hyp, thioproline, N-alkylglycine, N-alkylpentylglycine or N-alkylalanine, and Z₂ is —OH or —NH₂;

In certain embodiments contemplating compounds with structure of Formula II, exemplary N-alkyl groups for N-alkylglycine, N-alkylpentylglycine and N-alkylalanine include lower alkyl groups of 1 to about 6 carbon atoms, and in certain embodiments, of 1 to 4 carbon atoms.

In certain embodiments contemplating compounds with structure of Formula II, exemplary exendin agonist compounds include those wherein Xaa¹ is H is or Tyr. In certain embodiments, exemplary compounds are those compounds wherein Xaa² is Gly. In certain embodiments, exemplary compounds are those compounds wherein Xaa¹⁴ is Leu, pentylglycine or Met. In certain embodiments, exemplary compounds are those compounds wherein Xaa²⁵ is Trp or Phe. In certain embodiments, exemplary compounds are those compounds where Xaa⁶ is Phe or naphthylalanine; Xaa²² is Phe or naphthylalanine and Xaa²³ is Ile or Val.

In certain embodiments contemplating compounds with structure of Formula II, exemplary compounds are compounds wherein Xaa³¹, Xaa³⁶, Xaa³⁷ and Xaa³⁸ are independently selected from Pro, homoproline, thioproline and N-alkylalanine.

In certain embodiments contemplating compounds with structure of Formula II, Z₂ is —NH₂.

In certain embodiments, compounds of Formula II are provided wherein Xaa¹ is H is or Tyr; Xaa² is Gly; Xaa⁶ is Phe or naphthylalanine; Xaa¹⁴ is Leu, pentylglycine or Met; Xaa²² is Phe or naphthylalanine; Xaa²³ is Ile or Val; Xaa³¹, Xaa³⁶, Xaa³⁷ and Xaa³⁸ are independently selected from Pro, homoproline, thioproline or N-alkylalanine.

In certain embodiments, compounds include those of Formula II wherein Xaa¹ is H is or Arg; Xaa² is Gly or Ala; Xaa³ is Ala, Asp or Glu; Xaa⁵ is Ala or Thr; Xaa⁶ is Ala, Phe or naphthylalanine; Xaa⁷ is Thr or Ser; Xaa⁸ is Ala, Ser or Thr; Xaa⁹ is Asp or Glu; Xaa¹⁰ is Ala, Leu or pentylglycine; Xaa¹¹ is Ala or Ser; Xaa¹² is Ala or Lys; Xaa¹³ is Ala or Gln; Xaa¹⁴ is Ala, Leu or pentylglycine; Xaa¹⁵ is Ala or Glu; Xaa¹⁶ is Ala or Glu; Xaa¹⁷ is Ala or Glu; Xaa¹⁹ is Ala or Val; Xaa²⁰ is Ala or Arg; Xaa²¹ is Ala or Leu; Xaa²² is Phe or naphthylalanine; Xaa²³ is Ile, Val or tert-butylglycine; Xaa²⁴ is Ala, Glu or Asp; Xaa²⁵ is Ala, Trp or Phe; Xaa²⁶ is Ala or Leu; Xaa²⁷ is Ala or Lys; Xaa²⁸ is Ala or Asn; Z₁ is —OH, —NH₂, Gly-Z₂, Gly Gly-Z₂, Gly Gly Xaa³¹-Z₂, Gly Gly Xaa³¹ Ser-Z₂, Gly Gly Xaa³¹ Ser Ser-Z₂, Gly Gly Xaa³¹ Ser Ser Gly-Z₂, Gly Gly Xaa³¹Ser Ser Gly Ala-Z₂, Gly Gly Xaa³¹ Ser Ser Gly Ala Xaa³⁶-Z₂, Gly Gly Xaa³¹Ser Ser Gly Ala Xaa³⁶ Xaa³⁷-Z₂, Gly Gly Xaa³¹ Ser Ser Gly Ala Xaa³⁶ Xaa³⁷ Xaa³⁸-Z₂; Xaa³¹, Xaa³⁶, Xaa³⁷ and Xaa³⁸ being independently Pro homoproline, thioproline or N-methylalanine; and Z₂ is —OH or —NH₂; provided that no more than three of Xaa³, Xaa⁵, Xaa⁶, Xaa⁸, Xaa¹⁰, Xaa¹¹, Xaa¹², Xaa¹³, Xaa¹⁴, Xaa¹⁵, Xaa¹⁶, Xaa¹⁷, Xaa¹⁹, Xaa²⁰, Xaa²¹, Xaa²⁴, Xaa²⁵, Xaa²⁶, Xaa²⁷ and Xaa²⁸ are Ala.

In certain embodiments contemplating compounds with structure of Formula II, exemplary compounds include those compounds where Xaa¹⁴ is Leu, Ile, Val or pentylglycine, and Xaa²⁵ is Phe, Tyr or naphthylalanine.

In certain embodiments, exendin agonist compounds contemplated in the practice of the methods described herein comprise or include those described in Published Application WO99/25728, filed Nov. 13, 1998, entitled, “Novel Exendin Agonist Compounds,” which claims the benefit of U.S. Provisional Application No. 60/066,029, filed Nov. 14, 1997, and corresponding U.S. Pat. No. 7,220,721 issued May 22, 2007, each of which are herein incorporated by reference in their entireties, and for all purposes, which purposes include the exendin and exendin agonist compounds and formulations thereof taught therein. Included are compounds comprising the structure of Formula III (SEQ ID NO:_):

III
Xaa¹ Xaa² Xaa³ Xaa⁴ Xaa⁵ Xaa⁶ Xaa⁷ Xaa⁸ Xaa⁹ Xaa¹⁰
Xaa¹¹ Xaa¹² Xaa¹³ Xaa¹⁴ Xaa¹⁵ Xaa¹⁶ Xaa¹⁷ Ala
Xaa¹⁹ Xaa²⁰ Xaa²¹ Xaa²² Xaa²³ Xaa²⁴ Xaa²⁵ Xaa²⁶
Xaa²⁷ Xaa²⁸-Z₁;

wherein Xaa¹ is H is, Arg, Tyr, Ala, Norval, Val or Norleu; Xaa² is Ser, Gly, Ala or Thr; Xaa³ is Ala, Asp or Glu; Xaa⁴ is Ala, Norval, Val, Norleu or Gly; Xaa⁵ is Ala or Thr; Xaa⁶ is Phe, Tyr or naphthylalanine; Xaa⁷ is Thr or Ser; Xaa⁸ is Ala, Ser or Thr; Xaa⁹ is Ala, Norval, Val, Norleu, Asp or Glu; Xaa¹⁰ is Ala, Leu, Ile, Val, pentylglycine or Met; Xaa¹¹ is Ala or Ser; Xaa¹² is Ala or Lys; Xaa¹³ is Ala or Gln; Xaa¹⁴ is Ala, Leu, Ile, pentylglycine, Val or Met; Xaa¹⁵ is Ala or Glu; Xaa¹⁶ is Ala or Glu; Xaa¹⁷ is Ala or Glu; Xaa¹⁹ is Ala or Val; Xaa²⁰ is Ala or Arg; Xaa²¹ is Ala or Leu; Xaa²² is Phe, Tyr or naphthylalanine; Xaa²³ is Ile, Val, Leu, pentylglycine, tert-butylglycine or Met; Xaa²⁴ is Ala, Glu or Asp; Xaa²⁵ is Ala, Trp, Phe, Tyr or naphthylalanine; Xaa²⁶ is Ala or Leu; Xaa²⁷ is Ala or Lys; Xaa²⁸ is Ala or Asn; Z₁ is absent or —OH, —NH₂, Gly-Z₂, Gly Gly-Z₂, Gly Gly Xaa³¹-Z₂, Gly Gly Xaa³¹ Ser-Z₂, Gly Gly Xaa³¹ Ser Ser-Z₂, Gly Gly Xaa³¹ Ser Ser Gly-Z₂, Gly Gly Xaa³¹ Ser Ser Gly Ala-Z₂, Gly Gly Xaa³¹ Ser Ser Gly Ala Xaa³⁶-Z₂, Gly Gly Xaa³¹ Ser Ser Gly Ala Xaa³⁶ Xaa³⁷-Z₂, Gly Gly Xaa³¹ Ser Ser Gly Ala Xaa³⁶ Xaa³⁷ Xaa³⁸-Z₂ or Gly Gly Xaa³¹ Ser Ser Gly Ala Xaa³⁶ Xaa³⁷ Xaa³⁸ Xaa³⁹Z₂, wherein Xaa³¹, Xaa³⁶, Xaa³⁷ and Xaa³⁸ are independently Pro, homoproline, 3Hyp, 4Hyp, thioproline, N-alkylglycine, N-alkylpentylglycine or N-alkylalanine, and Z₂ is —OH or —NH₂;

In certain embodiments, an exendin agonist for use as described herein is an exendin analog. As used herein, an “analog” refers to a peptide whose sequence was derived from that of a base reference peptide, e.g., an exendin such as exendin-3 or exendin-4, or a GLP-1 receptor agonist, and includes insertions, substitutions, extensions, and/or deletions of the reference amino acid sequence. In certain embodiments, analogs have at least 50% amino acid sequence identity with the base peptide. In other embodiments, analogs have at least 60%, 70%, 80%, 90%, or 95% amino acid sequence identity with the base peptide. In other embodiments, analogs comprise not more than 20, not more that 15, not more than 10, not more than 5 or not more than 3 amino acids insertions, substitutions, extensions and/or deletions. Such analogs may comprise conservative or non-conservative amino acid substitutions including but limited to non-natural amino acids and L and D stereoisomeric forms.

As used herein, identity or sequence identity means the degree of sequence relatedness between polypeptide or polynucleotide sequences, as determined by the match between strings of such sequences. Identity can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York, 1993; Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J Applied Math, 48:1073 (1988). Methods to determine identity are designed to give the largest match between the sequences tested. Moreover, methods to determine identity are codified in publicly available programs. Computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, et al., Genome Analysis, 1: 543-559 (1997)). The BLAST X program is publicly available from NCBI and other sources (BLAST Manual, Altschul, S., et al., NCBI NLM NIH, Bethesda, Md. 20894; Altschul, S., et al., J. Mol. Biol., 215:403-410 (1990)).

In certain embodiments, the well-known Smith Waterman algorithm is used to determine identity. The choice of parameter values for matches, mismatches, and insertions or deletions is discretionary, although some parameter values have been found to yield more biologically realistic results than others. In one embodiment, the set of parameter values used for the Smith-Waterman algorithm is set forth in the “maximum similarity segments” approach, which uses values of 1 for a matched residue and −⅓ for a mismatched residue (a residue being either a single nucleotide or single amino acid). Waterman, Bull. Math. Biol. 46; 473 (1984). Insertions and deletions (indels), x, are weighted as xₖ=1⅓ k, where k is the number of residues in a given insert or deletion. Id.

The peptide or analog can be modified to further improve its agonist activity or other desirable property such as duration of action. Such modifications include, but are not limited to, derivatizations including phosphorylation, glycosylation, crosslinking, acylation, alkylation, proteolytic cleavage, linkage to an antibody molecule, membrane molecule or other ligand. The modified peptide or analog can be carboxy-terminal amidated or provided in its free carboxy-terminal hydroxy form.

The peptide or analog can also be further derivatized by chemical alterations such as amidation, glycosylation, acylation, sulfation, phosphorylation, acetylation, and cyclization. Such chemical alterations can be obtained through chemical or biochemical methodologies, as well as through in-vivo processes, or any combination thereof. Derivatives of the peptide or analog disclosed herein can also include conjugation to one or more polymers or small molecule substituents. One type of polymer conjugation is linkage or attachment of polyamino acids (e.g., poly-his, poly-arg, poly-lys, poly-glu, etc.) and/or fatty acid chains of various lengths to the N- or C-terminus or amino acid residue side chains of the peptide or analog. Small molecule substituents include short alkyls and constrained alkyls (e.g., branched, cyclic, fused, adamantyl), and aromatic groups.

In one embodiment, the peptide or analog thereof can be coupled to polyethylene glycol (PEG) by one of several strategies. Those skilled in the art will be able to utilize well-known techniques for linking one or more PEG polymers to the peptide or analog, described herein. Suitable PEG polymers typically are commercially available or can be made by techniques well know to those skilled in the art. In one embodiment, the polyethylene glycol polymers have molecular weights between 500 and 20,000 and can be branched or straight chain polymers. In other embodiments, the peptide or analog thereof are modified by the addition of polyamide chains of precise lengths as described, for example, in U.S. Pat. No. 6,552,167, the content of which is incorporated by reference in its entirety and for all purposes. In other embodiments, the peptide or analog thereof are modified by the addition of alkyl-PEG moieties as described in U.S. Pat. Nos. 5,359,030 and 5,681,811, the contents of which are incorporated by reference in their entirety and for all purposes.

An attachment of a PEG on an intact peptide or protein can be accomplished by coupling to amino, carboxyl or thiol groups. Such groups will typically be the N and C termini and on the side chains of such naturally occurring amino acids as lysine, aspartic acid, glutamic acid and cysteine. Since the compounds described herein can be prepared by solid phase peptide chemistry techniques, a variety of moieties containing diamino and dicarboxylic groups with orthogonal protecting groups can be introduced for conjugation to PEG.

The peptide or analog thereof can be linked to one or more macromolecules other than polyethylene glycol. Examples of such macromolecules include albumin, gelatin and antibodies. When the macromolecule is an antibody it can be a single chain antibody, an intact antibody, e.g. a catalytic antibody at the antibody's catalytic site via an appropriate hapten linker, or a fragment of an antibody, such as an Fc or Fab fragment. Further examples are described herein.

### GLP-1 Receptor Agonist Compounds

Some embodiments useful in the practice of the methods described herein contemplate exendin, exendin agonists and GLP-1 receptor agonists linked to a protraction protein. Accordingly, in some embodiments, exendin, exendin agonists and GLP-1 receptor agonist compounds contemplated herein include without limitation those described in Published Application WO 2005/058958, entitled “Novel GLP-1 Analogues Linked to Albumin-Like Agents,” and in U.S. application Ser. No. 11/454,348 (U.S. Publication No. US 2007/0093417), filed Jun. 16, 2006, both of which applications are herein incorporated by reference in their entireties and for all purposes, which purposes include the drug compounds are linked to a protraction protein and formulations thereof taught therein. Useful compounds in the context of linked protraction proteins include compounds with structure of Formula IV

GLP-1 agonist-L-RR-protraction protein IV

wherein “GLP-1 agonist” is a polypeptide which is an agonist of the human GLP-1 receptor as known in the art or as described herein, “L” is a linker connecting an amino acid side chain of the GLP-1 agonist or the C-terminal amino acid residue of the GLP-1 agonist with RR, “RR” is the remains of a reactive residue that has formed a covalent bond with an amino acid residue of the protraction protein, and the “protraction protein” is a protein having a molar weight of at least 5 kDa and having a plasma half-life of at least 24 hours in human plasma. In some embodiments, the protraction protein is synthesized by a non-mammalian organism. In some embodiments, the protraction protein is prepared synthetically.

In certain embodiments related to compounds with structure of Formula IV, the protraction protein is recombinant human serum albumin (HSA) (SEQ ID NO:_). In certain embodiments, the protraction protein is an HSA variant. In certain embodiments, the HSA variant has at least 80%, 90%, 95%, 96%, 97%, 98%, or even 99% amino acid sequence identity with respect to HSA. In certain embodiments, the HSA variant has reduced binding affinities towards copper and nickel as compared to the corresponding binding affinities of HSA towards copper and nickel. In certain embodiments, the protraction protein is an N-terminal fragment of HSA, or an analogue thereof. In certain embodiments, the protraction protein is a HSA variant comprising a modification of the Asp-Ala-His-Lys N-terminal sequence. In certain embodiments, the protraction protein comprises at least one deletion among the three N-terminal amino acid residues Asp-Ala-His. In certain embodiments, the protraction protein comprises an N-terminal extension, for example without limitation, Glu⁻³ Ala⁻²Glu⁻¹Phe⁰-HSA(1-585) or an N-terminal fragment thereof. In certain embodiments, the HSA variant is selected from the group consisting of HSA(2-585), HSA(3-585), HSA(4-585), Asp-Ala-HSA(4-585), Xaa³-HSA(1-585) where Xaa³ is an amino acid residue which has substituted the H is residue occupying position 3 in HSA, and N-terminal fragments thereof. In certain embodiments, the protraction protein comprises an amino acid sequence of from 60-200 amino acid residues, the amino acid sequence being identical to a fragment of HSA or a fragment of HSA with one or more (e.g., 1, 2, 3, 4, 5 or even more) amino acid substitutions and/or deletions.

In further embodiments contemplating compounds with structure of Formula IV, the protraction protein is the Fc portion of an immunoglobulin, an analogue or a fragment thereof. In certain embodiments, the GLP-1 agonist has at least 50% amino acid identity with either GLP-1(7-37) or Exendin-4(1-39). In certain embodiments, the GLP-1 agonist has at least 80%, at least 85%, at least 90%, at least 95%, or even 100% amino acid identity with either GLP-1(7-37) or Exendin-4(1-39).

In certain embodiments contemplating compounds with structure of Formula IV, the GLP-1 agonist comprises the amino acid sequence of Formula V (SEQ ID NO:_):

V
Xaa⁷-Xaa⁸-Glu-Gly-Thr-Phe-Thr-Ser-Asp-Xaa¹⁶-Ser-Xaa¹⁸-Xaa¹⁹-Xaa²⁰-Glu-Xaa²²-
Xaa²³-Ala-Xaa²⁵-Xaa²⁶-Xaa²⁷-Phe-Ile-Xaa³⁰-Trp-Leu-Xaa³³-Xaa³⁴-Xaa³⁵-Xaa³⁶-Xaa³⁷-
Xaa³⁸-Xaa³⁹-Xaa⁴⁰-Xaa⁴¹-Xaa⁴²-Xaa⁴³-Xaa⁴⁴-Xaa⁴⁵-Xaa⁴⁶

wherein Xaa⁷ is L-histidine, D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine; Xaa⁸ is Ala, D-Ala, Gly, Val, Leu, Ile, Lys, Aib, (1-aminocyclopropyl)carboxylic acid, (1-aminocyclobutyl)carboxylic acid, 1-aminocyclopentyl)carboxylic acid, (1-aminocyclohexyl)carboxylic acid, (1-aminocycloheptyl)carboxylic acid, or (1-aminocyclooctyl)carboxylic acid; Xaa¹⁶ is Val or Leu; Xaa¹⁸ is Ser, Lys or Arg; Xaa¹⁹ is Tyr or Gln; Xaa²⁰ is Leu or Met; Xaa²² is Gly, Glu or Aib; Xaa²³ is Gln, Glu, Lys or Arg; Xaa²⁵ is Ala or Val; Xaa²⁶ is Lys, Glu or Arg; Xaa²⁷ is Glu or Leu; Xaa³⁰ is Ala, Glu or Arg; Xaa³³ is Val or Lys; Xaa³⁴ is Lys, Glu, Asn or Arg; Xaa³⁵ is Gly or Aib; Xaa³⁶ is Arg, Gly or Lys; Xaa³⁷ is Gly, Ala, Glu, Pro, Lys, amide or is absent; Xaa³⁸ is Lys, Ser, amide or is absent. Xaa³⁹ is Ser, Lys, amide or is absent; Xaa⁴⁰ is Gly, amide or is absent; Xaa⁴¹ is Ala, amide or is absent; Xaa⁴² is Pro, amide or is absent; Xaa⁴³ is Pro, amide or is absent; Xaa⁴⁴ is Pro, amide or is absent; Xaa⁴⁵ is Ser, amide or is absent; Xaa⁴⁶ is amide or is absent; provided that if Xaa³⁸, Xaa³⁹, Xaa⁴⁰, Xaa⁴¹, Xaa⁴², Xaa⁴³, Xaa⁴⁴, Xaa⁴⁵ or Xaa⁴⁶ is absent then each amino acid residue downstream is also absent.

In certain embodiments, in compounds with structure of Formula IV, the GLP-1 agonist is protected against DPP-IV by substitutions known in the art. In certain embodiments, the GLP-1 agonist is substituted at position 8. In certain embodiments, the GLP-1 agonist comprises an Aib residue (aminoisobutyric acid) at position 8. In certain embodiments, the amino acid residue in position 7 of the GLP-1 peptide (e.g., the N-terminal of GLP-1(7-37)) is selected from the group consisting of D-histidine, desamino-histidine, 2-amino-histidine, β-hydroxy-histidine, homohistidine, N^{α}-acetyl-histidine, α-fluoromethyl-histidine, α-methyl-histidine, 3-pyridylalanine, 2-pyridylalanine and 4-pyridylalanine. In certain embodiments, the GLP-1 agonist comprises no more than 12, no more than 6, no more than 4, or no more than 2 amino acid residues which have been exchanged, added or deleted as compared to GLP-1(7-37) or Exendin-4(1-39). In certain embodiments, the GLP-1 agonist comprises no more than 4 amino acid residues which are not encoded by the genetic code.

In further embodiments contemplating compounds with structure of Formula IV, the GLP-1 agonist is selected from the group consisting of [Arg³⁴]GLP-1(7-37), [Arg^{26,34}]GLP-1(7-37)Lys, [Lys³⁶Arg^{26,34}]GLP-1(7-36), [Aib^{8,22,35}]GLP-1(7-37), [Aib^{8,35}]GLP-1(7-37), [Aib^{8,22}]GLP-1(7-37), [Aib^{8,22,35}Arg^{26,34}]GLP-1 (7-37)Lys, [Aib^{8,35} Arg^{26,34}]GLP-1 (7-37)Lys, [Aib^{8,22}Arg^{26,34}]GLP-1 (7-37)Lys, [Aib^{8,22,35}Arg^{26,34}]GLP-1(7-37)Lys, [Aib^{8,35} Arg^{26,34}]GLp-1 (7-37)Lys, [Aib^{8,22,35}Arg²⁶]GLP-1(7-37)Lys, [Aib^{8,35}Arg²⁶]GLP-1 (7-37)Lys, [Aib^{8,22}Arg²⁶]GLP-1(7-37)Lys, [Aib^{8,22,35}Arg³⁴]GLP-1(7-37)Lys, [Aib^{8,35}Arg³⁴]GLP-1(7-37)Lys, [Aib-8,22Arg³⁴]GLP-1 (7-37)Lys, [Aib^{8,22,35}Ala³⁷]GLP-1(7-37)Lys, [Aib^{8,35}Ala³⁷]GLP-1 (7-37)Lys, [Aib^{8,22}Ala³⁷]GLP-1(7-37)Lys, [Aib^{8,22,35}Lys³⁷]GLP-1(7-37), [Aib^{8,35}Lys³⁷]GLP-1 (7-37) and [Aib^{8,22}Lys³⁷]GLP-1(7-37). In certain embodiments, the GLP-1 agonist is exendin-4(1-39). In certain embodiments, the GLP-1 agonist is ZP-10 with structure [Ser¹⁸Lys³⁹]Exendin-4(1-39)LysLysLysLysLys-amide.

In further embodiments contemplating compounds with structure of Formula IV, the GLP-1 agonist is attached to the moiety “-L-RR-protraction protein” via the side chain of the amino acid residue in position 23, 26, 34, 36 or 38 (i.e., relative to GLP-1(7-37)), corresponding to position 17, 20, 28, 30 or 32 relative to Exendin-4(1-39). In further embodiments, the GLP-1 agonist is attached to the moiety “-L-RR-protraction protein” via the side chain of the C-terminal amino acid residue. In further embodiments, the GLP-1 agonist is attached to the moiety “-L-RR-protraction protein” via the side chain of an amino acid residue selected from arginine, lysine, cysteine, glutamic acid, aspartic acid, histidine, serine, threonine and tyrosine.

In further embodiments contemplating compounds with structure of Formula IV, the linker L is selected from the group consisting of the bivalent connecting chemical groups amide, amine, thioethers, ethers, urethanes, carbamates, hydrazines, oximes, oxazolidines or thiazolidines.

Exemplary compounds with structure of Formula IV contemplated by the methods, described herein include, without limitation, GLP-1 agonist —C(═O)CH₂O(CH₂)₂O(CH₂)₂—RR-protraction protein, GLP-1 agonist —C(═O)(CH₂)ₙ(OCH₂CH₂)ₘ—RR-protraction protein, GLP-1 agonist —S(═O)₂(CH₂)ₙ(OCH₂CH₂)ₘ—RR-protraction protein, GLP-1 agonist —CH₂(CH₂)ₙ(OCH₂CH₂)ₘ—RR-protraction protein, GLP-1 agonist —C(═O)O(CH₂)ₙ(OCH₂CH₂)ₘ—RR-protraction protein, wherein n is an integer in the range from 0 to 10, and m is an integer in the range from 0 to 100.

Further exemplary compounds with structure of Formula IV contemplated by the methods described herein include, without limitation, GLP-1 agonist-L-NC(═O)CH₂— sulphur in cysteine residue in protraction protein, GLP-1 agonist-L-S(═O)₂(CH₂)₂— sulphur in cysteine residue in protraction protein, GLP-1 agonist-L-NC(═O)CH₂— sulphur in cysteine residue in protraction protein, and GLP-1 agonist-L-RR′-sulphur in cysteine residue in protraction protein, wherein RR′ is 2,5-dioxo-pyrrolidine-1,3-diyl.

Further exemplary compounds with structure of Formula V contemplated herein include, without limitation, S-γ³⁴-(1-{2-[2-(2-([Lys³²]-exendin-(1-39)amide-N-ε³²-yl)acetyloxyethoxy)ethylcarbamoyl]ethyl}-2,5-dioxo-pyrrolidin-3-yl) Albumin, wherein Albumin is recombinant Albagen (New Century Pharma, recombinant HSA(2-585)); S-γ³⁴-(1-{2-[2-(2-([Lys²⁰)]-exendin-(1-39)amide-N-ε-²⁰-yl)acetyloxyethoxy)ethylcarbamoyl]ethyl}-2,5-dioxo-pyrrolidin-3-y-1) Albumin, wherein Albumin is recombinant Albagen; S-γ³⁴-(1-{2-[2-(2-([Arg¹², Lys²⁷]-exendin-(1-39)amide-N-ε²⁷-yl)acetyloxyethoxy)ethylcarbamoyl]ethyl}-2,5-dioxo-pyrrolidin-3-yl) Albumin, wherein Albumin is recombinant Albagen; and S-γ³⁴-(1-{2-[2-(2-([Arg^{12,27}, Lys³²]-exendin-(1-39)amide-N-ε³²-yl)acetyloxyethoxy)ethylcarbamoyl]ethyl}-2,5-dioxo-pyrrolidin-3-yl) Albumin, wherein Albumin is recombinant Albagen.

Some embodiments of the methods described herein contemplate exendins, exendin agonists, and GLP-1 receptor agonists linked to a protracting tag. Accordingly, in some embodiments, compounds contemplated herein include without limitation those described in Published Application WO 2007/068718, entitled “Polypeptide Protracting Tags,” filed Dec. 13, 2006, which application is herein incorporated by reference in its entirety and for all purposes, which purposes include the protracting tags, and the exendins, exendin agonists, and GLP-1 receptor agonists linked to protracting tags and formulations thereof taught therein. The protracting tags taught therein can be used with any of the exendin, exendin agonists or GLP-1 receptor agonists described herein. Useful compounds in this context include compounds with structure of Formula VI

R—W¹—P(Y¹)(Y²)—W²-A-C(═O)—W³-molecule VI

wherein A represents —(CH₂)₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆alkyl, C₁₋₆-alkoxy or carboxy; R represents C₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl; or C₃₋₁₀-cycloalkyl- optionally substituted with one or more substituent(s) selected from the group consisting of aryl, haloaryl, cyanoaryl, heteroaryl, C₃₋₁₀-cycloalkyl, carboxyl, 5-tetrazolyl, acylaminosulfonyl, sulfonylaminocarbonyl, and a straight or branched C₁₋₆alkyl; W¹ and W² independently are —O—, —CH₂— or —S—; Y¹ is —OH or —SH; Y² is ═O or ═S; W³ is a bond or a spacer; and the term “molecule” represents a fragment obtained by formal abstraction of a hydrogen atom from an amino group, a hydroxy group, or a mercapto group of an exendin, exendin agonist, or GLP-1 receptor agonist; with the proviso that at least either A represents —(CH₂)₁₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy, or R represents C₁₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆alkyl.

In certain embodiments contemplating compounds with structure of Formula VI, spacer W³ is selected from the group consisting of oligo(ethylene glycol), an amino acid or a combination thereof.

In certain embodiments contemplating compounds with structure of Formula VI, the molecule comprises the amino acid sequence with structure of Formula VII (SEQ ID NO:_):

VII
Xaa⁷-Xaa⁸-Glu-Gly-Thr-Phe-Thr-Ser-Asp-Xaa¹⁶-Ser-Xaa¹⁸-Xaa¹⁹-Xaa²⁰-Glu-Xaa²²-
Xaa²³-Ala-Xaa²⁵-Xaa²⁶-Xaa²⁷-Phe-Ile-Xaa³⁰-Trp-Leu-Xaa³³-Xaa³⁴-Xaa³⁵-Xaa³⁶-Xaa³⁷-
Xaa³⁸-Xaa³⁹-Xaa⁴⁰-Xaa⁴¹-Xaa⁴²-Xaa⁴³-Xaa⁴⁴-Xaa⁴⁵-Xaa⁴⁶

wherein Xaa⁷ is L-histidine, D-histidine, desamino-histidine, 2-amino-3-(2-aminoimidazol-4-yl)propionic acid, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine; Xaa⁸ is Ala, Gly, Val, Leu, He, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid; Xaa¹⁶ is Val or Leu; Xaa¹⁸ is Ser, Lys or Arg; Xaa¹⁹ is Tyr or Gln; Xaa²⁰ is Leu or Met; Xaa²² is Gly, Glu or Aib; Xaa²³ is Gln, Glu, Lys or Arg; Xaa²⁵ is Ala or Val; Xaa²⁶ is Lys, Glu or Arg; Xaa²⁷ is Glu or Leu; Xaa³⁰ is Ala, Glu or Arg; Xaa³³ is Val or Lys; Xaa³⁴ is Lys, Glu, Asn or Arg; Xaa³⁵ is Gly or Aib; Xaa³⁶ is Arg, Gly or Lys; Xaa³⁷ is Gly, Ala, Glu, Pro, Lys, amide or is absent; Xaa³⁸ is Lys, Ser, amide or is absent; Xaa³⁹ is Ser, Lys, amide or is absent; Xaa⁴⁰ is Gly, amide or is absent; Xaa⁴¹ is Ala, amide or is absent; Xaa⁴² is Pro, amide or is absent; Xaa⁴³ is Pro, amide or is absent; Xaa⁴⁴ is Pro, amide or is absent; Xaa⁴⁵ is Ser, amide or is absent; Xaa⁴⁶ is amide or is absent; provided that if Xaa³⁹, Xaa⁴⁰, Xaa⁴¹, Xaa⁴², Xaa⁴³, Xaa⁴⁴, Xaa⁴⁵ or Xaa⁴⁶ is absent then each amino acid residue downstream is also absent.

In certain embodiments contemplating compounds with structure of Formula VI, the molecule is selected from GLP-1(7-35), GLP-1(7-36), GLP-1(7-36)-amide, GLP-1(7-37), GLP-1(7-38), GLP-1(7-39), GLP-1(7-40), GLP-1(7-41) or an analogue thereof. In certain embodiments, the molecule is selected from the group consisting of Arg³⁴GLP-1(7-37), Lys³⁸Arg^{26,34}GLP-1(7-38), Lys³⁵Arg^{26,34}GLP-1(7-3 8)—OH, Lys³⁶Arg^{26,34}GLP-1(7-36), Aib^{8,22,35}GLP-1(7-37), Aib^{8,35}GLP-1(7-37), Aib^{8,22}GLP-1(7-37), Aib^{8,22,35}Arg^{26,34}Lys³⁸GLP-1(7-38), Aib^{8,35}Arg^{26,34}Lys³⁸GLP-1(7-38), Aib^{8,22}Arg^{26,34}Lys³⁸GLP-1(7-38), Aib^{8,22,35}Arg^{26,34}Lys³⁸GLP-1(7-38), Aib-8,35Arg^{26,34}Lys³⁸GLP-1(7-38), Aib^{8,22,35}Arg²⁶Lys³⁸GLP-1(7-38), Aib^{8,35}Arg²⁶Lys³⁸GLP-1(7-38), Aib^{8,22}Arg²⁶Lys³⁸GLP-1(7-38), Aib^{8,22,35}Arg³⁴Lys³⁸GLP-1(7-38), Aib^{8,35}Arg³⁴Lys³⁸GLP-1(7-38), Aib^{8,22}Arg³⁴Lys³⁸GLP-1(7-38), Aib^{8,22,35}Ala³⁷Lys³⁸GLP-1(7-38), Aib^{8,35}Ala³⁷Lys³⁸GLP-1(7-38), Aib^{8,22}Ala³⁷Lys³⁸GLP-1(7-38), Aib^{8,22,35}Lys³⁷GLP-1(7-37), Aib^{8,35}Lys³⁷GLP-1(7-37), and Aib^{8,22}Lys³⁷GLP-1(7-38).

Further exemplary compounds with structure of Formula VI include N-ε²⁶-((S)-4-[16-{(hydroxy)(octadecyloxy)phosphoryloxy}hexadecanoylamino]-4-carboxybutyryl)[Aib⁸,Arg³⁴]GLP-1(7-37), N-ε²⁶-((S)-4-[16-{(hydroxy)(pentyloxy) phosphoryloxy}hexadecanoylamino]-4-carboxybutyryl)[Aib⁸, Arg³⁴]GLP-1(7-37), N-ε²⁶-((S)-4-[16-{(hydroxy)(dodecyloxy)phosphoryloxy}hexadecanoylamino]-4-carboxybutyryl)[Aib⁸, Arg³⁴]GLP-1(7-37), N-ε²⁶-((S)-4-[16-{(hydroxy)(methoxy)phosphoryl}nonadecanoylamino]-4-carboxybutyryl)[Aib⁸, Arg³⁴]GLP-1(7-37), and N-ε²⁶-(3-(2-{2-[2-(hexadecyloxy-hydroxy-phosphoryloxy)-ethoxy]-ethoxy}-ethoxy)-propionyl)-[Aib⁸,Arg³⁴]GLP-I (7-37).

Some embodiments of the methods described herein contemplate GLP-1 receptor agonist which are GLP-1 analogs having a modification of at least one non-proteogenic amino acid residues in positions 7 and/or 8 relative to GLP-1(7-37), and which GLP-1 receptor agonist is acylated with a moiety to the lysine residue in position 26, wherein the acylation moiety comprises at least two acidic groups, and wherein the acylation is optionally via a non-natural amino acid hydrophilic linker. Accordingly, in some embodiments, GLP-1 receptor agonist compounds contemplated in the practice of the methods described herein include without limitation those described in Published Application WO 2006/097537, filed Mar. 20, 2006, entitled “Acylated GLP-1 Compounds”, which application is herein incorporated by reference in its entirety and for all purposes, which purposes include the GLP-1 receptor agonists and formulations thereof taught therein. Useful compounds for the methods described herein include GLP-1 analogs having non-proteogenic amino acid residues in positions 7 and/or 8 relative to GLP-1(7-37) which is acylated with a moiety to the lysine residue in position 26, and where said moiety comprises at least two acidic groups, wherein one such acidic group is attached terminally. In certain embodiments, the moiety attached at position 26 comprises a hydrophilic linker. In certain embodiments, the hydrophilic linker comprises at least five non-hydrogen atoms wherein 30-5-% are either N or O. In certain embodiments, the moiety attached at position 26 comprises an albumin binding moiety separated from the peptide by the hydrophilic linker. The term “albumin binding moiety” as used herein refers to a moiety which binds non-covalently to human serum albumin. In certain embodiments, the albumin binding moiety is a linear or branched lipophilic moiety containing 4-40 carbon atoms having a distal acid group.

In this context, exemplary GLP-1 receptor agonist compounds contemplated in the practice of the methods described herein include, without limitation, [N^{ε}-(17-carboxy-heptadecanoyl)-Lys²⁶,Aib⁸,Arg³⁴]GLP-1-(7-37), [N^{ε}-(19-carboxynonadecanoyl)-Lys²⁶,Aib⁸,Arg³⁴]GLP-1-(7-37), [N^{ε}-(4-{[N-(2-carboxyethyl)-N-(15-carboxypentadecanoyl)amino]methyl}benzoyl)-Lys²⁶,Arg³⁴]GLP-1-(7-37), [N^{ε}-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl]-Lys²⁶,Aib⁸,Arg³⁴]GLP-1-(7-37), [N^{ε}-[2-(2-[2-(2-[2-(2-[4-(19-carboxynonadecanoylamino)-4(S)-carboxybutyrylamino]ethoxy)ethoxy]acetylamino) ethoxy]ethoxy)acetyl]-Lys²⁶,Aib⁸,Arg³⁴]GLP-1-(7-37), [N^{ε}-[2-(2-[2-(2-[2-(2-[4-(17-carboxyheptadecanoylamino)-4(S)-carboxybutyrylamino]ethoxy)ethoxy]acetylamino) ethoxy]ethoxy)acetyl]-Lys²⁶,3-(4-imidazolyl)propionyl⁷,Arg³⁴]GLP-1-(7-37), [N^{ε}-[2-(2-[2-(2-[2-(2-[4-(17-carboxyheptadecanoylamino)-(carboxymethyl-amino)acetylamino]ethoxy)ethoxy]acetylamino)ethoxy]ethoxy)acetyl]-Lys²⁶,Aib⁸,Arg³⁴]GLP-1-(7-37), [N^{ε}-[2-(2-[2-(2-[2-(2-[4-(17-carboxyheptadecanoylamino)-3(S)-sulfopropionylaminogethoxy)ethoxy]acetylamino) ethoxy]ethoxy)acetyl]-Lys²⁶,Aib⁸,Arg³⁴]GLP-1-(7-37), [N^{ε}-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl]-Lys²⁶,Gly8,Arg³⁴]GLP-1-(7-37), [N^{ε}-[2-(2-[2-(2-[2-(2-[4-(17-carboxyheptadecanoylamino)-4(S)-carboxybutyrylamino]ethoxy)ethoxy]acetylamino) ethoxy]ethoxy)acetyl]-Lys²⁶, Aib⁸,Arg³⁴]GLP-1-(7-37)-amide, [N^{ε}-[2-(2-[2-(2-[2-(2-[4-(17-carboxyheptadecanoylamino)-4(S)-carboxybutyrylamino]ethoxy)ethoxy]acetylamino) ethoxy]ethoxy)acetyl]-Lys²⁶,Aib⁸,Arg³⁴,Pro³⁷)GLP-1-(7-3 7)amide, [Aib⁸,N^{ε}-{2-(2-(2-(2-[2-(2-(4-(pentadecanoylamino)-4-carboxybutyrylamino)ethoxy)ethoxy]acetyl)ethoxy)ethoxy)acetyl}-Lys²⁶,Arg³⁴]GLP-1(7-37)-OH, N^{ε}-[2-(2-[2-(2-[2-(2-[4-{[N-(2-carboxyethyl)-N-(17-carboxyheptadecanoyl)amino]methyl}benzoyl)amino]ethoxy)ethoxy]acetylamino)ethoxy]ethoxy)acetyl]-Lys²⁶,Aib⁸,Arg³⁴]GLP-1 (7-37), [N-α⁷-formyl, N^{ε}-[2-(2-[2-(2-[2-(2-[4-(17-carboxyheptadecanoylamino)-4(S)-carboxybutyrylamino]ethoxy)ethoxy]acetylamino) ethoxy]ethoxy)acetyl]-Lys²⁶,Arg³⁴ GLP-1(7-37), [N^{ε}-[2-(2-[2-(2-[2-(2-[4-(17-carboxyheptadecanoylamino)-4(S)-carboxybutyrylamino]ethoxy)ethoxy]acetylamino) ethoxy]ethoxy)acetyl]-Lys²⁶,Aib⁸,Glu²²,Arg³⁴]GLP-1-(7-37), [N^{ε}-{3-[2-(2-{2-[2-(2-{2-[2-(2-[4-(15-(N—((S)-1,3-dicarboxypropyl)carbamoyl)pentadecanoylamino)-(S)-4-carboxybutyrylamino]ethoxy)ethoxy]ethoxy}ethoxy)ethoxy]ethoxyethoxy)ethoxy]propionyl}-Lys²⁶,Aib⁸,Arg³⁴]GLP-1(7-37), [N^{ε}-[2-(2-[2-(2-[2-(2-[4-{[N-(2-carboxyethyl)-N-(17-carboxyheptadecanoyl)amino]methyl}benzoyl)amino](4(S)-carboxybutyrylamino)ethoxy)ethoxy]acetylamino)ethoxy]ethoxy)acetyl]-Lys²⁶,Aib⁸,Arg³⁴]GLP-1(7-37), [N^{ε}-{(S)-4-carboxy-4-((S)-4-carboxy-4-((S)-4-carboxy-4-((S)-4-carboxy-4-(19-carboxynonadecanoylamino)butyrylamino) butyrylamino)butyrylamino)butyrylamino}-Lys²⁶,Aib⁸,Arg³⁴]GLP-1-(7-37), [N^{ε}-4-(17-carboxyheptadecanoylamino)-4(S)-carboxybutyryl-Lys²⁶,Aib⁸,Arg³⁴]GLP-1-(7-37), [N^{ε}-{3-[2-(2-{2-[2-(2-{2-[2-(2-[4-(17-carboxyheptadecanoylamino)-4(S)-carboxybutyrylamino]ethoxy)ethoxy]ethoxy}ethoxy)ethoxy]ethoxy}ethoxy)ethoxy]propionyl}-Lys²⁶,Aib⁸,Arg³⁴]GLP-4(7-37), [N^{ε}-{2-(2-(2-(2-[2-(2-(4-(17-carboxyheptadecanoylamino)-4-carboxybutyrylamino)ethoxy)ethoxy]acetyl)ethoxy)ethoxy)acetyl)}-Lys²⁶ Aib^{8,22,27,30,35},Arg³⁴,Pro³⁷]GLP-1(7-37)amide, [N^{ε}-[2-(2-[2-[4-(21-carboxyuneicosanoylamino)-4(S)-carboxybutyrylamino]ethoxy]ethoxy)acetyl]-Lys²⁶,Aib⁸,Arg³⁴]GLP-1-(7-37), [N^{ε}-[2-(2-[2-(2-[2-(2-[4-(21-carboxyuneicosanoylamino)-4(S)-carboxybutyrylamino]ethoxy)ethoxy]acetylamino)ethoxy]ethoxy)acetyl]-Lys²⁶,Aib⁸,Arg³⁴]GLP-1-(7-37), and [N-α¹-formyl-N^{ε}-[2-(2-[2-(2-[2-(2-[4-(19-carboxynonadecanoylamino)-4(S)-carboxybutyrylamino]ethoxy)ethoxy]acetylamino)ethoxy]ethoxy)acetyl]-Lys²⁶,Arg³⁴]GLP-1-(7-37).

Some embodiments of the methods described herein contemplate exendin, exendin agonists or GLP-1 receptor agonist, particularly analogs of GLP-1 and exendin, acylated with a diacid, and further in which the drug compound is optionally stabilized against DPP-IV by modification of at least one amino acid residue in positions 7-8 (relative to GLP-1(7-37)). Accordingly, in some embodiments, exendin agonist or GLP-1 receptor agonist compounds contemplated in the practice of the methods described herein include without limitation those described in Published Application WO 2006/037810, filed Oct. 7, 2005, entitled “Protracted GLP-1 Compounds,” which application is herein incorporated by reference in its entirety and for all purposes, which purposes include the GLP-1 receptor agonist compounds and formulations thereof taught therein. Useful compounds in this context include acylated GLP-1 receptor agonists which are stabilized against DPP-IV activity by modification of at least one amino acid residue in positions 7-8, and wherein acylation is afforded with a diacid attached directly to the C-terminal amino acid residue of the GLP-1 analog. In certain embodiments, the diacid is attached to the ε-amino group of a lysine residue of the GLP-1 receptor agonist. In certain embodiments, the GLP-1 receptor agonist has an extended C-terminal wherein the compound comprises an amino acid residue in position 38 relative to the sequence GLP-1 (7-37). In certain embodiments, the diacid is attached to Lys³⁸. In certain embodiments, the diacid is a dicarboxylic acid. In certain embodiments, the acylation group is a straight-chain or branched alkane α,ω-dicarboxylic acid. In certain embodiments, the acylation group has a structure selected from HOOC—(CH₂)₁₄CO—, HOOC—(CH₂)₁₅CO—, HOOC—(CH₂)₁₆CO—, HOOC—(CH₂)₁₇CO—, and HOOC—(CH₂)₁₈CO—. In certain embodiments, the GLP-1 receptor agonist comprises a modification of the N-terminal L-histidine in position 7 of the GLP-1 (7-37) sequence. In certain embodiments, the GLP-1 receptor agonist comprises imidazopropionyl⁷, α-hydroxy-histidine⁷ or N-methyl-histidine⁷, D-histidine⁷, desamino-histidine⁷, 2-amino-histidine⁷, β-hydroxy-histidine⁷, homohistidine⁷, N^{α}-acetyl-histidine⁷, α-fluoromethyl-histidine⁷, α-methyl-histidine⁷, 3-pyridylalanine⁷, 2-pyridylalanine⁷ or 4-pyridylalanine⁷. In certain embodiments, the GLP-1 receptor agonist comprises a substitution of the L-alanine in position 8 of the GLP-1(7-37) for another amino acid. In certain embodiments, the GLP-1 receptor agonist comprises Aib⁸, Gly⁸, Val⁸, Ile⁸, Leu⁸, Ser⁸ or Thr⁸. In certain embodiments, the GLP-1 receptor agonist comprises a substitution of the L-alanine in position 8 of the GLP-1(7-37) sequence for (1-aminocyclopropyl) carboxylic acid, (1-aminocyclobutyl) carboxylic acid, (1-aminocyclopentyl) carboxylic acid, (1-aminocyclohexyl) carboxylic acid, (1-aminocycloheptyl) carboxylic acid, or (1-aminocyclooctyl) carboxylic acid. In certain embodiments, the GLP-1 receptor agonist comprises no more than 15, no more than 10, no more than 6, or no more than 4 amino acid residues which have been exchanged, added, or deleted as compared with GLP-1(7-37). In certain embodiments, the GLP-1 receptor agonist useful in the practice of the methods described herein comprises only one lysine residue.

Exemplary acylated GLP-1 receptor agonist compounds contemplated in the practice of the methods described herein include, without limitation, [Aib⁸,Arg^{26,34}]GLP-1(7-37)Lys(17-carboxyheptadecanoyl)-OH, [Gly⁸,Arg^{26,34,36}]GLP-1(7-37)Lys(17-carboxyheptadecanoylamino)-OH, [α-hydroxydesamino-His⁷,Gly⁸,Arg^{26,34}]GLP-1 (7-37)Lys(17-carboxyheptadecanoyl)-OH, [Gly⁸,Glu^{22,23,30},Arg^{18,26,34}]GLP-1(7-37)Lys(17-carboxyheptadecanoyl)-NH₂, and [Gly⁸,Arg^{26,34}]GLP-1(7-37)Lys(19-carboxynonadecanoyl)-OH.

Some embodiments of the methods described herein contemplate exendin agonist compounds wherein one amino acid has been exchanged by a lysine residue which lysine residue is acylated with a diacid. Accordingly, in some embodiments, exendin agonist compounds contemplated in the practice of the methods described herein include without limitation those described in Published Application WO 2006/037811, filed Oct. 7, 2005, entitled “Protracted Exendin-4 Compounds,” which application is herein incorporated by reference in its entirety and for all purposes, which purposes include the exendin agonist compounds and formulations thereof taught therein. Useful exendin agonist compounds for the practice of the methods described herein include exendin-4 analogs wherein one amino acid has been exchanged by a lysine residue, which lysine residue is acylated with a diacid. In certain embodiments, the exendin-4 analog comprises an acylated lysine residue in position 20 or 32. In certain embodiments, the diacid is a dicarboxylic acid. In certain embodiments, the acylation group is a straight-chain or branched alkane α,ω-dicarboxylic acid. In certain embodiments, the acylation group has the structure HOOC—(CH₂)ₙCO—, wherein n is 12 to 20. In certain embodiments, the acylation group has a structure selected from HOOC—(CH₂)₁₄CO—, HOOC—(CH₂)₁₅CO—, HOOC—(CH₂)₁₆CO—, HOOC—(CH₂)₁₇CO—, and HOOC—(CH₂)₁₈CO—. In certain embodiments, exendin agonist compounds useful in the practice of the methods described herein are acylated exendin-4 analogs comprising no more than 15, no more than 10, no more than 6, or no more than 4 amino acid residues which have been exchanged, added or deleted as compared to exendin-4(1-39) (SEQ ID NO:_). In certain embodiments, the exendin agonist useful in the practice of the methods described herein comprises only one lysine residue.

In the context of protracted exendin-4 compounds, exemplary exendin agonist compounds contemplated in the practice of the methods described herein include, without limitation, [N^{ε}-(17-carboxyheptadecanoic acid)Lys²⁰]exendin-4(1-39)amide, [N^{ε}-(17-carboxy-heptadecanoyl)Lys³²]exendin-4(1-39)amide, [Desamino-His¹,N^{ε}-(17-carboxy-heptadecanoyl)Lys²⁰)exendin-4(1-39)amide, [Arg^{12,27},NLe¹⁴,N^{ε}-(17-carboxy-heptadecanoyl)Lys³²]exendin-4(1-39)amide, [N^{ε}-(19-carboxynonadecanoylamino)Lys²⁰]exendin-4(1-39)-amide, [N^{ε}-(15-carboxypentadecanoylamino)Lys²⁰]exendin-4(1-39)-amide, [N^{ε}-(13-carboxytridecanoylamino)Lys²⁰]exendin-4(1-39)-amide, and [N^{ε}-(11-carboxyundecanoylamino)Lys²⁰]exendin-4(1-39)-amide.

Some embodiments of the methods described herein contemplate amino acid functionalization with a lipophilic substituent. Accordingly, in certain embodiments, GLP-1 receptor agonist compounds contemplated for the practice of the methods described herein include without limitation those described in U.S. Pat. No. 6,569,832, filed Nov. 10, 2000, entitled “Inhibition of Beta Cell Degeneration,” which claims the benefit of U.S. Provisional Application No. 60/166,800, filed Nov. 22, 1999, and U.S. Provisional Application No. 60/185,845, filed Feb. 29, 2000, all of which applications are herein incorporated by reference in their entireties and for all purposes, which purposes include peptides having lipophilic substitution and formulations thereof taught therein. In certain embodiments, compounds are contemplated with the structure of Formula VIII (SEQ ID NO:_):

VIII
His⁷-Xaa⁸-Xaa⁹-Gly¹⁰-Xaa¹¹-Phe¹²-Thr¹³-Xaa¹⁴-Asp¹⁵-Xaa¹⁶-Xaa¹⁷-
Xaa¹⁸-Xaa¹⁹-Xaa²⁰-Xaa²¹-Xaa²²-Xaa²³-Xaa²⁴-Xaa²⁵-Xaa²⁶-Xaa²⁷-Phe²⁸-
Ile²⁹-Xaa³⁰-Xaa³¹-Xaa³²-Xaa³³-Xaa³⁴-Xaa³⁵-Xaa³⁶-Xaa³⁷-Xaa³⁸-
Xaa³⁹-Xaa⁴⁰-Xaa⁴¹-Xaa⁴²-Xaa⁴³-Xaa⁴⁴-Xaa⁴⁵

wherein Xaa⁸ is Ala, Gly, Ser, Thr, Leu, Ile, Val, Glu, Asp, Met, or Lys, Xaa⁹ is Glu, Asp, or Lys, Xaa¹¹ is Thr, Ala, Gly, Ser, Leu, Ile, Val, Glu, Asp, or Lys, Xaa¹⁴ is Ser, Ala, Gly, Thr, Leu, Ile, Val, Glu, Asp, or Lys, Xaa¹⁶ is Val, Ala, Gly, Ser, Thr, Leu, Ile, Tyr, Glu, Asp, or Lys, Xaa¹⁷ is Ser, Ala, Gly, Thr, Leu, Ile, Val, Glu, Asp, or Lys, Xaa¹⁸ is Ser, Ala, Gly, Thr, Leu, Ile, Val, Glu, Asp, or Lys, Xaa¹⁹ is Tyr, Phe, Trp, Glu, Asp, or Lys, Xaa²⁰ is Leu, Ala, Gly, Ser, Thr, Leu, Ile, Val, Glu, Asp, or Lys, Xaa²¹ is Glu, Asp, or Lys, Xaa²² is Gly, Ala, Ser, Thr, Leu, Ile, Val, Glu, Asp, or Lys, Xaa²³ is Gln, Asn, Arg, Glu, Asp, or Lys, Xaa²⁴ is Ala, Gly, Ser, Thr, Leu, Ile, Val, Arg, Glu, Asp, or Lys, Xaa²⁵ is Ala, Gly, Ser, Thr, Leu, Ile, Val, Glu, Asp, or Lys, Xaa²⁶ is Lys, Arg, Gln, Glu, Asp, or H is, Xaa²⁷ is Glu, Asp, or Lys, Xaa³⁰ is Ala, Gly, Ser, Thr, Leu, Ile, Val, Glu, Asp, or Lys, Xaa³¹ is Trp, Phe, Tyr, Glu, Asp, or Lys, Xaa³² is Leu, Gly, Ala, Ser, Thr, Ile, Val, Glu, Asp, or Lys, Xaa³³ is Val, Gly, Ala, Ser, Thr, Leu, Ile, Glu, Asp, or Lys, Xaa³⁴ is Lys, Arg, Glu, Asp, or H is, Xaa³⁵ is Gly, Ala, Ser, Thr, Leu, Ile, Val, Glu, Asp, or Lys, Xaa³⁶ is Arg, Lys, Glu, Asp, or H is, Xaa³⁷ is Gly, Ala, Ser, Thr, Leu, Ile, Val, Glu, Asp, or Lys, or is deleted, Xaa³⁸ is Arg, Lys, Glu, Asp, or H is, or is deleted, Xaa³⁹ is Arg, Lys, Glu, Asp, or H is, or is deleted, Xaa⁴⁰ is Asp, Glu, or Lys, or is deleted, Xaa⁴¹ is Phe, Trp, Tyr, Glu, Asp, or Lys, or is deleted, Xaa⁴² is Pro, Lys, Glu, or Asp, or is deleted, Xaa⁴³ is Glu, Asp, or Lys, or is deleted, Xaa⁴⁴ is Glu, Asp, or Lys, or is deleted, and Xaa⁴⁵ is Val, Glu, Asp, or Lys, or is deleted, or a C₁₋₆-ester, amide, C₁₋₆-alkylamide, or C₁₋₆-dialkylamide thereof and/or a pharmaceutically acceptable salt thereof, provided that (i) when the amino acid at position 37, 38, 39, 40, 41, 42, 43 or 44 is deleted, then each amino acid downstream (i.e., toward the C-terminus) of the amino acid is also deleted, (ii) the derivative of the GLP-1 analog contains only one or two Lys, (iii) the ε-amino group of one or both Lys is optionally substituted with a lipophilic substituent optionally via a spacer, and (iv) the total number of different amino acids between the derivative of the GLP-1 analog and the corresponding native form of GLP-1 or fragment thereof does not exceed six.

In certain embodiments, in compounds with structure of Formula VIII, Xaa⁸ is Val. In certain embodiments, the ε-amino group of one Lys residue is substituted with a lipophilic substituent optionally via a spacer. In certain embodiments, the ε-amino group of more than one Lys residue is substituted with a lipophilic substituent optionally via a spacer. In certain embodiments, the ε-amino group of two Lys residues are independently substituted with a lipophilic substituent optionally via a spacer.

Exemplary compounds with structure of Formula VIII include, without limitation: Lys³⁴-(N^{ε}-(γ-glutamyl(N^{α}-tetradecanoyl)))GLP-1(7-37); Arg^{26,34}, Lys⁸(N^{ε}-(γ-glutamyl(N^{α}-hexadecanoyl)))GLP-1 (7-37); Arg³⁴, Lys²⁶ (N^{ε}(γ-glutamyl(N^{α}-dodecanoyl)))GLP-1(7-37); Arg³⁴, Lys²⁶ (N^{ε}(β-alanyl(N^{α}-hexadecanoyl)))GLP-1(7-37); Arg³⁴, Lys²⁶(N^{ε}(α-glutamyl(N^{α}-hexadecanoyl)))GLP-1(7-37); Arg³⁴, Lys²⁶ (N^{ε}(piperidinyl-4-carbonyl (N-hexadecanoyl)))GLP-1 (7-37); and Arg³⁴, Lys²⁶(N^{ε}(γ-glutamyl(N^{α}-decanoyl)))GLP-1(7-37).

Some embodiments of the methods described herein further contemplate amino acid functionalization with a lipophilic substituent. Accordingly, in certain embodiments, GLP-1 receptor agonist compounds contemplated for the practice of the methods described herein include without limitation compounds described in U.S. Pat. No. 6,268,343 filed Feb. 26, 1999, entitled “Derivatives of GLP-1 Analogs,” which is herein incorporated by reference in its entirety and for all purposes, which purposes include the lipophilic substituted peptides and formulations thereof taught therein. In certain embodiments, compounds are contemplated with the structure of Formula IX (SEQ ID NO:_):

IX
His⁷-Xaa⁸-Xaa⁹-Gly¹⁰-Xaa¹¹-Phe¹²-Thr¹³-Xaa¹⁴-Asp¹⁵-Xaa¹⁶-Xaa¹⁷-
Xaa¹⁸-Xaa¹⁹-Xaa²⁰-Xaa²¹-Xaa²²-Xaa²³-Xaa²⁴-Xaa²⁵-Xaa²⁶-Xaa²⁷-Phe²⁸-
Ile²⁹-Xaa³⁰-Xaa³¹-Xaa³²-Xaa³³-Xaa³⁴-Xaa³⁵-Xaa³⁶-Xaa³⁷

wherein Xaa⁸ is Ala, Xaa⁹ is Glu, Xaa¹¹ is Thr, Xaa¹⁴ is Ser, Xaa¹⁶ is Val, Xaa¹⁷ is Ser, Xaa¹⁸ is Ser, Xaa¹⁹ is Tyr, Xaa²⁰ is Leu, Xaa²¹ is Glu, Xaa²² is Gly, Xaa²³ is Gln, Xaa²⁴ is Ala, Xaa²⁵ is Ala, Xaa²⁶ is Lys, Xaa²⁷ is Glu, Xaa³⁰ is Ala, Xaa³¹ is Trp, Xaa³² is Leu, Xaa³³ is Val, Xaa³⁴ is Arg, Xaa³⁵ is Gly, Xaa³⁶ is Arg, and Xaa³⁷ is Gly, wherein (a) the ε-amino group of Lys²⁶ is substituted with a lipophilic substituent, optionally via a spacer, (b) the lipophilic substituent is (i) CH₃(CH₂)ₙCO— wherein n is 6, 8, 10, 12, 14, 16, 18, 20 or 22, (ii) HOOC(CH₂)ₘCO— wherein m is 10, 12, 14, 16, 18, 20 or 22, or (iii) lithocholoyl, and (c) the spacer is (i) an unbranched alkane α,ω-dicarboxylic acid group having from 1 to 7 methylene groups, (ii) an amino acid residue except Cys, or (iii) γ-aminobutanoyl.

In certain embodiments, in the GLP-1 derivative of Formula IX, the lipophilic substituent is linked to the ε-amino group of Lys via a spacer. In certain embodiments, the spacer is γ-glutamyl. In certain embodiments, the spacer is β-asparagyl. In certain embodiments, the spacer is glycyl. In certain embodiments, the spacer is γ-aminobutanoyl. In certain embodiments, the spacer is β-alanyl.

In certain embodiments, the compound with structure of Formula IX is Lys²⁶ (N^{ε}-tetradecanoyl), Arg³⁴-GLP-1(7-37). In certain embodiments, the compound with structure of Formula IX is Lys²⁶(N^{ε}-(ω-carboxynonadecanoyl)), Arg³⁴-GLP-1(7-37). In certain embodiments, the compound with structure of Formula IX is Lys²⁶(N^{ε}-(ω-carboxyheptadecanoyl)), Arg³⁴-GLP-1(7-37). In certain embodiments, the compound with structure of Formula IX is Lys²⁶(N^{ε}-(ω-carboxyundecanoyl)), Arg³⁴-GLP-1(7-37). In certain embodiments, the compound with structure of Formula IX is Lys²⁶ (N^{ε}(ω-carboxypentadecanoyl)), Arg³⁴-GLP-1(7-37). In certain embodiments, the compound with structure of Formula IX is Lys²⁶(N^{ε}-lithocholoyl), Arg³⁴-GLP-1(7-37). In certain embodiments, the compound with structure of Formula IX is Lys²⁶(N^{ε}-(γ-glutamyl(N^{α}-hexadecanoyl))), Arg³⁴-GLP-1(7-37). In certain embodiments, the compound with structure of Formula IX is Lys²⁶(N^{ε}-(γ-glutamyl(N^{α}-tetradecanoyl))), Arg³⁴-GLP-1(7-37). In certain embodiments, the compound with structure of Formula IX is Lys²⁶(N^{ε}-(γ-glutamyl(-lithocholoyl))), Arg³⁴-GLP-1(7-37). In certain embodiments, the compound with structure of Formula IX is Lys²⁶(N^{ε}-(γ-glutamyl(N^{α}-octadecanoyl))), Arg³⁴-GLP-1(7-37). In certain embodiments, the compound with structure of Formula IX is Lys²⁶(N^{ε}-decanoyl), Arg³⁴-GLP-1(7-37). In certain embodiments, the compound with structure of Formula IX is Lys²⁶(N^{ε}-hexadecanoyl), Arg³⁴-GLP-1(7-37). In certain embodiments, the compound with structure of Formula IX is Lys²⁶(N^{ε}-octanoyl), Arg³⁴-GLP-1(7-37). In certain embodiments, the compound with structure of Formula IX is Lys²⁶(N^{ε}-dodecanoyl), Arg³⁴-GLP-1(7-37). In certain embodiments, the compound with structure of Formula IX is Lys²⁶(N^{ε}(γ-aminobutyroyl-(N^{γ}-hexadecanoyl))), Arg³⁴-GLP-1(7-37). In certain embodiments, the compound with structure of Formula IX is Lys²⁶(N^{ε}-(γ-D-glutamyl(N^{α}-hexadecanoyl))), Arg³⁴-GLP-1(7-37). In certain embodiments, the compound with structure of Formula IX is Lys²⁶(N^{ε}-(γ-glutamyl(N^{α}-dodecanoyl))), Arg³⁴-GLP-1(7-37). In certain embodiments, the compound with structure of Formula IX is Lys²⁶(N^{ε}-(β-alanyl(N^{α}-hexadecanoyl))), Arg³⁴-GLP-1(7-37). In certain embodiments, the compound with structure of Formula IX is Lys²⁶(N^{ε}-(α-glutamyl(N^{α}-hexadecanoyl))), Arg³⁴-GLP-1(7-37). In certain embodiments, the compound with structure of Formula IX is Lys²⁶(N^{ε}-(γ-glutamyl(N^{α}-decanoyl))), Arg³⁴-GLP-1(7-37).

Some embodiments of the methods described herein contemplate conjugates of an exendin, exendin agonist or GLP-1 receptor agonist to a macromolecule. Useful conjugated peptides for the methods described herein include exendins, exendin agonists and GLP-1 receptor agonists and derivatives thereof conjugated to a macromolecule. In certain embodiments, the macromolecule is a peptide, a blood-protein-binding peptide, a hormone, a polypeptide, an albumin, a polyamino acid, a fatty acyl group, a diacid, a water soluble polymer, an immunoglobulin or an immunoglobulin fragment, or a catalytic antibody or fragment thereof. In certain embodiments, the macromolecule is a blood protein. In one embodiment, the macromolecule is albumin. Methods known in the art can be used to link an exendin, exendin agonist or GLP-1 receptor agonist peptide to a macromolecule. In certain embodiments, the peptide is linked to albumin according to any technique known to those of skill in the art. In some embodiments, the peptide is modified to include a reactive group which can react with available reactive functionalities on albumin to form a covalent linkage. In certain embodiments, two of said macromolecules are present and are selected from the group consisting of a peptide, a blood-protein-binding peptide, a hormone, a polypeptide, an albumin, a polyamino acid, a fatty acyl group, a diacid, a water soluble polymer, an immunoglobulin or an immunoglobulin fragment, or a catalytic antibody or fragment thereof. Accordingly, in some embodiments, compounds contemplated in the practice of the methods described herein include without limitation those described in Published Application WO 2007/053946, entitled “Method of Treating Diabetes and/or Obesity with Reduced Nausea Side Effects using an Insulinotropic Peptide Conjugated to Albumin,” which application is herein incorporated by reference in its entirety and for all purposes, which purposes include the exendin, exendin agonist or GLP-1 receptor agonist conjugates and formulations thereof taught therein.

In some embodiments, compounds conjugated with blood proteins include modified peptides comprising a reactive group which reacts with amino groups, hydroxyl groups or thiol groups on blood proteins to form stable covalent bonds. Exemplary reactive groups include NHS(N-hydroxysuccinimide), sulfo-NHS(N-hydroxy-sulfosuccinimide), MBS(maleimide-benzoyl-succinimide), GMBS (gamma-maleimid-butyryloxy succinimide ester), and MPA (maleimidopropionic acid), and the like. Exemplary reactive group containing peptides include without limitation GLP-1(1-36)-Lys³⁷(ε-MPA)-NH₂; GLP-1(1-36)-Lys³⁷(ε-AEEA-AEEA-MPA)-NH₂; GLP-1(7-36)-Lys³⁷(ε-MPA)-NH₂; GLP-1(7-36)-Lys³⁷(ε-AEEA-AEEA-MPA)-NH₂, D-Ala2 GLP-1(7-36)-Lys³⁷(ε-MPA)-NH₂; Exendin-4 (1-39)-Lys⁴⁰(ε-MPA)-NH₂; Exendin-4(1-39)-Lys⁴⁰(ε-AEEA-AEEA-MPA)-NH₂; Exendin-3(1-39)-Lys⁴⁰(ε-MPA)-NH₂; Exendin-3(1-39)-Lys⁴⁰(ε-AEEA-AEEA-MPA)-NH₂; Lys²⁶(ε-MPA)GLP-1(7-36)-NH₂; GLP-1(7-36)-EDA-MPA; and Exendin-4(1-39)-EDA-MPA. “AEEA” refers to the linking group [2-(2-amino)ethoxy)]ethoxy acetic acid. “EDA” refers to ethylenediamine. Methods for preparing compounds containing reactive groups, and for the covalent attachment of peptides with blood proteins are well known in the art.

In some embodiments, the conjugated peptide comprises albumin Cys³⁴ thiol covalently linked to a [2-[2-[2-maleimidopropionamido(ethoxy)ethoxy]acetamide linker on the e-amino of a lysine of the exendin, exendin agonist or GLP-1 receptor agonist peptide. In certain embodiments, the lysine has been added to the native peptide sequence. In certain embodiments, the lysine has been added to the carboxy terminus of the peptide. In certain embodiments, the peptide is selected from the group consisting of GLP-1, exendin-3, and exendin-4, and analogs and derivatives thereof including each of the species described herein. In certain embodiments, the peptide is exendin-4(1-39). In certain embodiments, the albumin is human serum albumin. In certain embodiments, the albumin is recombinant serum albumin as known in the art. In certain embodiments, the albumin is recombinant human serum albumin. In certain embodiments, the conjugated peptide comprises recombinant human serum albumin Cys³⁴ thiol covalently linked to a [2-[2-[2 maleimidopropionamido(ethoxy)ethoxy]acetamide linker on the ε-amino of the carboxy terminal lysine of exendin-4(1-39)Lys.

Some embodiments of the methods described herein contemplate an exendin or exendin agonist or GLP-1 receptor agonist coupled with polyethylene glycol (PEG). Accordingly, in some embodiments, GLP-1 receptor agonist compounds contemplated in the practice of the methods described herein include without limitation those described in Published Application WO 2006/124529, filed May 11, 2006, entitled “GLP-1 Pegylated Compounds,” which application is herein incorporated by reference in its entirety and for all purposes, which purposes include the pegylated compounds useful for the methods described herein and formulation thereof taught therein. Useful compounds contemplated in this context include compounds with structure of Formula X

X
His-Xaa⁸-Glu-Gly-Thr-Phe-Thr-Ser-Asp-Val-Ser-Ser-Tyr-Leu-Glu-
Xaa²²-Gln-Ala˜Ala-Lys-Glu-Phe-He-Ala-Trp-Leu-Xaa³³-Lys-Gly-
Gly-Pro-Ser-Ser-Gly-Ala-Pro-Pro-Pro-Cys⁴⁵-Xaa⁴⁶

wherein Xaa⁸ is D-Ala, Gly, Val, Leu, Ile, Ser, or Thr; Xaa²² is Gly, Glu, Asp, or Lys; Xaa³³ is Val or Ile Xaa⁴⁶ is Cys or Cys-NH₂, and wherein one PEG molecule is covalently attached to Cys⁴⁵ and one PEG molecule is covalently attached to Cys⁴⁶ or Cys⁴⁶-NH₂. The term “pegylated” when referring to a GLP-1 receptor agonist refers to a GLP-1 receptor agonist that is chemically modified by covalent attachment of two molecules of polyethylene glycol or a derivative thereof. Furthermore, it is intended that the term “PEG” refers to polyethylene glycol or a derivative thereof as are known in the art (see, e.g., U.S. Pat. Nos. 5,445,090; 5,900,461; 5,932,462; 6,436,386; 6,448,369; 6,437,025; 6,448,369; 6,495,659; 6,515,100 and 6,514,491). In one embodiment of pegylated GLP-1 receptor agonist compounds, PEG (or a derivative thereof) is covalently attached to two introduced cysteine residues in the GLP-1 receptor agonist. In a further embodiment the two introduced cysteine residues in the GLP-1 receptor agonist are at position 45 and 46. In certain embodiments, the PEG polymers have molecular weights between 500 and 100,000 daltons, or between 5,000 and 40,000 daltons, or between 20,000 and 60,000 daltons, or between 20,000 and 40,000 daltons, and may be linear or branched molecules, and may be polyethylene glycol derivatives as described in the art. In still a further embodiment the PEG is a 20 kilodalton linear methoxy PEG maleimide.

Some embodiments of the methods described herein contemplate analogs of GLP-1 useful as GLP-1 receptor agonists. Accordingly, in some embodiments, GLP-1 receptor agonist compounds contemplated herein include without limitation those described in Published Application WO 98/43658, filed Mar. 25, 1998, entitled “Glucagon-like Peptide-1 Analogs,” which application is herein incorporated by reference in its entirety and for all purposes, which purposes include the GLP-1 receptor agonists and formulations thereof taught therein. In this context, compound useful in the practice of the methods described herein include GLP-1 receptor agonists with structure of Formula XI (SEQ ID NO:_):

XI
R¹-X-Glu-Gly-Thr-Phe-Thr-Ser-Asp-Val-Ser-Ser-Tyr-
Leu-Y-Gly-Gln-Ala-Ala-Lys-Z-Phe-Ile-Ala-Trp-Leu-
Val-Lys-Gly-Arg-R²

or a pharmaceutically acceptable salt thereof, wherein R¹ is selected from the group consisting of His, D-histidine, desamino-histidine, 2-amino-histidine, β-hydroxy-histidine, homohistidine, α-fluoromethylhistidine, and α-methyl-histidine; X is selected from the group consisting of Met, Asp, Lys, Thr, Leu, Asn, Gln, Phe, Val and Tyr; Y and Z are independently selected from the group consisting of Glu, Gln, Ala, Thr, Ser, and Gly, and; R² is selected from the group consisting of NH₂ and Gly-OH; provided that, if R¹ is H is, X is Ala or Val, Y is Glu and Z is Glu, then R² is NH₂. Exemplary compounds with structure of Formula XI include Met⁸GLP-1(7-36)NH₂ and Thr⁸GLP-1(7-37)-OH.

Some embodiments of the methods described herein contemplate analogs of GLP-1 useful as GLP-1 receptor agonists. Accordingly, in some embodiments, GLP-1 receptor agonist compounds contemplated in the practice of the methods described herein include without limitation those described in U.S. Pat. No. 5,512,549, filed Oct. 18, 1994, entitled “Glucagon-like Insulinotropic Peptide Analogs, Compositions, and Methods of Use,” which application is herein incorporated by reference in its entirety and for all purposes, which purposes include the GLP-1 receptor agonists and formulations thereof described therein. In this context, compounds useful in the practice of the methods described herein include GLP-1 receptor agonists with structure of Formula XII (SEQ ID NO:_):

XII
R¹-Ala-Glu-Gly-Thr-Phe-Thr-Ser-Asp-Val-
Ser-Ser-Tyr-Leu-Glu-Gly-Gln-Ala-Ala-Xaa¹-
Glu-Phe-Ile-Ala-Trp-Leu-VaI-Lys(R²)-Gly-Arg-R³

wherein R¹ is selected from the group consisting of 4-imidazopropionyl, 4-imidazoacetyl, or 4-imidazo-α,α-dimethylacetyl; R² is selected from the group consisting of C₆-C₁₀ unbranched acyl, or is absent; R³ is selected from the group consisting of Gly-OH or NH₂; and, Xaa¹ is Lys or Arg. In certain embodiments, R¹ is 4-imidazopropionyl, R² is C₈ unbranched acyl, R³ is Gly-OH, and Xaa¹ is Arg. In certain embodiments, R² is absent. In certain embodiments, R¹ is 4-imidazoacetyl and R² is C₈ unbranched acyl. In certain embodiments, R¹ is 4-imidazo-α,α-dimethyl-acetyl and R² is C₈ unbranched acyl. Exemplary compounds with the structure of Formula XII include, without limitation, [Arg²⁶]GLP-1(8-37)OH, [Lys³⁴-N^{ε}-Octanoyl]GLP-1(7-37))OH, N-Imidazopropionyl-GLP-1 (8-37)OH, N-Imidazopropionyl-[Arg²⁶]GLP-1(8-37)OH, N-imidazopropionyl-[Arg²⁶, Lys³⁴-N^{ε}-Octanoyl]GLP-1(8-37))OH, N-imidazoacetyl-[Arg²⁶]GLP-1 (8-37))OH, N-[Imidazole-α,α-dimethyl-acetyl]GLP-1(8-37)OH, and N-imidazoacetyl-GLP-1(8-36)NH₂.

In some embodiments, exendin and exendin agonist and GLP-1 receptor agonist compounds contemplated in the practice of the methods described herein include without limitation those prepared as in or described in Published Application WO 02/098348, filed May 21, 2002, entitled “GLP-1 Formulations with Protracted Time Action,” and U.S. Pat. No. 7,144,863, which is a national stage filing thereof, which applications are herein incorporated by reference in their entireties and for all purposes, including the particles and their making described therein. Provided for the methods herein is a composition comprising particles wherein the particles are comprised of an exendin, exendin agonist or GLP-1 receptor agonist complexed with a basic polypeptide. The basic polypeptide can be selected from the group consisting of polylysine, polyarginine, polyornithine, protamine, putrescine, spermine, spermidine, and histone. The mass ratio of compound to basic polypeptide in the composition is between about 4:1 and about 10:1. The mean number diameter of the particles is between 1 μm and 5 μm. In one embodiment the compound comprises any one of the exendin, exendin agonists or GLP-1 receptor agonists described herein or in WO 02/098348.

“Basic polypeptides” include but are not limited to basic proteins or polyamines. Examples of basic proteins or polyamines are polylysine, polyarginine, polyornithine, protamine, putrescine, spermine, spermidine, and histone. In one embodiment basic polypeptides are polyarginine, protamine, polylysine, polyaspartic acid, polyglutamic acid, and polyornithine. In another embodiment the basic peptide is polylysine, polyarginine, and protamine. In yet a further embodiment is protamine. Protamine is the generic name of a group of strongly basic proteins present in sperm cell nuclei in salt like combination with nucleic acids. Commercially available protamines can be isolated from mature fish sperm and are usually obtained as the sulfate. The peptide composition of a specific protamine may vary depending on which family, genera or species of fish it is obtained from. Protamine from salmon or trout can be separated into two, three, or more main fractions of proteins that may be separated further. The different parent peptides consist of about 30 amino acids of which more than 20 are arginines. The average molecular weight of protamine is about 4,300. Commercially available protamine sulfate is approximately 80% protamine. “Particle” in the context of compounds described in the present specification refers to a solid material complex comprising an exendin, exendin agonist or GLP-1 receptor agonist compound and a basic polypeptide. The particles optionally comprise divalent metal ions. The particles are comprised of either crystalline or amorphous material or a mixture of crystalline and amorphous material. A crystalline particle is a particle comprised primarily of individual or clusters of microcrystals, rods, needles, or plates or mixtures thereof. The crystalline particles can be comprised of small clusters of plate-like microcrystals. Further, the crystalline particles can be homogeneous in size and shape (unimodal) and appear as small clusters of plate-like microcrystals. The particles of the present invention have a number diameter that ranges from about 0.5 μm to about 12 μm. Particles can have a number diameter that ranges from about 1 μm to about 5 μM. Further, the particles can have a number diameter that ranges from about 1 μm to about 3 μm. The number mean diameter of the crystalline particles in a composition can be from about 1 μM to about 5 μm. Further, the number mean diameter of the crystalline particles in a composition can be from about 1 μm to about 3 μm. Even further, the number mean diameter of the crystalline particles in a composition is from about 3 μm to about 5 μm. Yet even further, the number mean diameter of the crystalline particles in a composition is from about 4 μm to about 5 μm. Yet even further, 90% of the particles in the composition are less than 12 μm. Further yet, 90% of the particles in the composition are less than 9 μm, and in a further embodiment most are less than 7 μm. The number diameters can be determined using a Coulter Multisizer II (Coulter Electronics Limited, Luton, Beds, England). The Coulter Multisizer uses an electrical sensing zone technique. Particle size, volume, and surface area distributions are calculated based on measurable changes in electrical resistance produced by non-conductive particles suspended in an electrolyte. An amorphous particle for the purposes of the present invention refers to a particle comprising a precipitate, but lacking matter in a crystalline state and a definable form or structure as determined by polarized light microscopy.

Accordingly, the particles formed from an exendin, exendin agonist or GLP-1 receptor agonist, such as a GLP-1 compound described herein or in WO 02/098348 and a basic polypeptide can be added together at a ratio between about 4:1 and about 10:1 (weight per weight) (w/w), further at a ratio between about 5:1 and about 10:1 (w/w), even further at a ratio between about 6:1 and about 10:1 (w/w), and yet even her at a ratio between about 7:1 and about 9:1 (w/w) (compound:basic polypeptide). Additionally, in one embodiment the composition is a mixture of particle preparations wherein particles formed from mixing, precipitating, or crystallizing a compound and a basic polypeptide at one ratio are mixed with particles formed from mixing, precipitating, or crystallizing a compound and a basic polypeptide at another ratio. The particles are soluble in that the amount of drug compound in particle form will dissolve in phosphate buffered saline (PBS) in a given desired period of time. Typically, a dried particle preparation is suspended in phosphate buffered saline (PBS) such that the final concentration of drug compound is 1 mg/mL. The resulting suspension is gently stirred for one hour at room temperature. Solubility is then determined by measuring the concentration of the drug compound released, such as by UV absorbance measurements or HPLC. In one embodiment the solubility range for the particles in PBS can range from about 0.5 mg/ml to about 0.1 mg/ml.

In one embodiment the particle composition can be administered by a pulmonary route, to a lower airway of the patient, or inhaled through the mouth of the patient. The particle composition can be delivered from an inhalation device suitable for pulmonary administration and capable of depositing the composition in the lungs of the patient, for example the device can be a nebulizer, a metered-dose inhaler, a dry powder inhaler, or a sprayer.

Embodiments of the methods described herein contemplate an exendin, exendin agonist or GLP-1 receptor agonist linked to a macromolecule, such as a peptide, a blood-protein-binding peptide, a hormone, a polypeptide, an albumin, a polyamino acid, a fatty acyl group, a diacid, a water soluble polymer, an immunoglobulin or an immunoglobulin fragment, such as an Fc fragment, or a catalytic antibody or fragment thereof. Such macromolecules provide a longer circulating half-life compared to the unconjugated compound, while maintaining bioactivity and often improving solubility compared to the unconjugated drug compound. Accordingly, compounds contemplated in the practice of the methods described herein include without limitation those described in Published Application WO 02/46227, filed Nov. 29, 2001, entitled “GLP-1 Fusion Proteins,” and related published applications WO 2004/110472 and WO 2006/068910, which applications are herein incorporated by reference in their entirety and for all purposes, which purposes include the macromolecules described therein and their exendin, exendin agonist and GLP-1 receptor agonist conjugates and methods of making and formulating. In this context the heterologous fusion proteins (conjugates) comprise an exendin, exendin agonist or a GLP-1 receptor agonist compound fused to or conjugated to human albumin, a human albumin analog, a human albumin fragment, an immunoglobulin, the Fc portion of an immunoglobulin, an analog of the Fc portion of an immunoglobulin, or a fragment of the Fc portion of an immunoglobulin. Typically, the C-terminus of the compound can be fused directly, or fused via a peptide linker, to the N-terminus of the macromolecule, such as an albumin or Fc protein. In the case of a catalytic macromolecule, such as a catalytic antibody, the compound is conjugated to a linker that is recognized by the catalytic antibody hapten biding site, which then covalently reacts with the linker, thus conjugating the compound at a precise location on the antibody. These heterologous fusion proteins are biologically active and have an increased half-life compared to native exendin or GLP-1 or their unconjugated analog or derivative form. The macromolecule can have a long circulating half-life, such as human serum albumin or the Fe portion of an immunoglobulin, or in any event the conjugate will have a longer circulating half-life than the unconjugated drug.

In one embodiment the heterologous fusion proteins comprise an exendin, exendin agonist or a GLP-1 receptor compound fused to human albumin, a human albumin analog, a human albumin fragment, the Fe portion of an immunoglobulin, an analog of the Fe portion of an immunoglobulin, or a fragment of the Fe portion of an immunoglobulin. The C-terminus of the drug compound may be fused directly, or fused via a peptide linker, to the N-terminus of an albumin, IgG or Fe protein. These heterologous fusion proteins are biologically active and have an increased half-life compared to the unconjugated compound.

Accordingly, in one embodiment are the formulas of heterologous proteins DE-L-M and DG-L-M, where DE is an exendin or exendin agonist and DG is a GLP-1 receptor agonist, such as Val8-GLP-1(7-36) or analog thereof, and wherein L is an optionally present linker and M is a macromolecule. In one embodiment the heterologous fusion protein comprises a first polypeptide DE or DG with a N-terminus and a C-terminus fused to a second polypeptide M with a N-terminus and a C-terminus wherein the first polypeptide is exendin or exendin agonist or is a GLP-1 receptor agonist and the second polypeptide is selected from the group consisting of a) the Fe portion of an immunoglobulin; b) an analog of the Fe portion of an immunoglobulin; and c) fragments of the Fe portion of an immunoglobulin, and wherein the C-terminus of the first polypeptide is fused to the N-terminus of the second polypeptide. In yet another embodiment, the heterologous fusion protein comprises a first polypeptide DE OR DG with a N-terminus and a C-terminus fused to a second polypeptide M with a N-terminus and a C-terminus wherein the first polypeptide is a GLP-1 compound and the second polypeptide is selected from the group consisting of a) the Fe portion of an immunoglobulin; b) an analog of the Fe portion of an immunoglobulin; and c) fragments of the Fe portion of an immunoglobulin, and wherein the C-terminus of the first polypeptide is fused to the N-terminus of the second polypeptide via a peptide linker. Accordingly, in another embodiment of the formulates DE-L-M and DG-L-M, the second polypeptide M is selected from the group consisting of a) human albumin; b) human albumin analogs; and c) fragments of human albumin, and wherein the C-terminus of the first polypeptide DE or DG is fused to the N-terminus of the second polypeptide. In yet another embodiment, M is selected from the group consisting of 20 a) human albumin; b) human albumin analogs; and c) fragments of human albumin, and wherein the C-terminus of the first polypeptide is fused to the N-terminus of the second polypeptide via a peptide linker. For example, the peptide linker can be a) a glycine rich peptide; b) a peptide having the sequence [Gly-Gly-Gly-Gly-Ser]ₙ where n is 1, 2, 3, 4, 5, or 6; and c) a peptide having the sequence [Gly-Gly-Gly-Gly-Ser]₃. The Fc portion of an Ig can be selected from the group consisting of: IgG1, IgG2, IgG3, IgG4, IgE, IgA, IgD, or IgM. The Fc portion of an Ig can be selected from the group consisting of: IgG1, IgG2, IgG3, and IgG4. The Fc portion can be an IgG4 immunoglobulin. In still further embodiment the blood protein, albumin or IgG is human. In yet another embodiment the Fc portion comprises the hinge, CH2, and CH3 domains. Further examples of linkers for use with any of the macromolecules including FC and albumin fusions, include a linker that is a G-rich peptide linker having the sequence Gly-Gly-Gly-Gly-Ser-Gly-Gly-Gly-Gly-Ser-Gly-Gly-Gly-Gly-Ser (SEQ ID NO:_). Other examples of linkers include, but are not limited to, Gly-Ser-Gly-Gly-Gly Gly-Ser-Gly-Gly-Gly-Gly-Ser-Gly-Gly-Gly-Gly-Ser-Gly-Gly-Gly-Gly-Ser (SEQ ID NO:_); Gly-Gly-Gly-Gly-Ser-Gly-Gly-Gly-Gly-Ser-Gly-Gly-Gly-Gly-Ser-Gly-Gly Gly-Gly-Ser-Gly-Gly-Gly-Gly-Ser-Gly-Gly-Gly-Gly-Ser (SEQ ID NO:_); Asp-Ala Ala-Ala-Lys-Glu-Ala-Ala-Ala-Lys-Asp-Ala-Ala-Ala-Arg-Glu-Ala-Ala-Ala-Arg-Asp Ala-Ala-Ala-Lys (SEQ ID NO:_) and Asn-Val-Asp-His-Lys-Pro-Ser-Asn-Thr-Lys-Val Asp-Lys-Arg (SEQ ID NO:_).

Thus, in one embodiment the drug compounds described herein and in WO02/46227 can be fused directly or via a peptide linker to the Fc portion of an immunoglobulin. Immunoglobulins are molecules containing polypeptide chains held together by disulfide bonds, typically having two light chains and two heavy chains. In each chain, one domain (V) has a variable amino acid sequence depending on the antibody specificity of the molecule. The other domains (C) have a rather constant sequence common to molecules of the same class. As used herein, the Fc portion of an immunoglobulin has the meaning commonly given to the term in the field of immunology. Specifically, this term refers to an antibody fragment which is obtained by removing the two antigen binding regions (the Fab fragments) from the antibody. One way to remove the Fab fragments is to digest the immunoglobulin with papain protease. Thus, the Fe portion is formed from approximately equal sized fragments of the constant region from both heavy chains, which associate through non-covalent interactions and disulfide bonds. The Fc portion can include the hinge regions and extend through the CH2 and CH3 domains to the C-terminus of the antibody. Representative hinge regions for human and mouse immunoglobulins can be found in Antibody Engineering, A Practical Guide, Borrebaeck, C. A. K., ed., W.H. Freeman and Co., 1992, the teachings of which are herein incorporated by reference. The Fc portion can further include one or more glycosylation sites. There are five types of human immunoglobulin Fc regions with different effect or and pharmacokinetic properties: IgG, IgA, IgM, IgD, and IgE. IgG is the most abundant immunoglobulin in serum. IgG also has the longest half-life 5 in serum of any immunoglobulin (23 days). Unlike other immunoglobulins, IgG is efficiently recirculated following binding to an Fc receptor. There are four IgG subclasses G1, G2, G3, and G4, each of which has different effect or functions. G1, G2, and G3 can bind Clq and fix complement while G4 cannot. Even though G3 is able to bind Clq more efficiently than G1, G1 is more effective at mediating complement-directed cell lysis. G2 fixes complement very inefficiently. The Clq binding site in IgG is located at the carboxy terminal region of the CH2 domain. All IgG subclasses are capable of binding to Fc receptors (CD16, CD32, CD64) with G1 and G3 being more effective than G2 and G4. The Fc receptor binding region of IgG is formed by residues located in both the hinge and the carboxy terminal regions of the CH2 domain. IgA can exist both in a monomeric and dimeric form held together by a J-chain. IgA is the second most abundant Ig in serum, but it has a half-life of only 6 days. IgA has three effect or functions. It binds to an IgA specific receptor on macrophages and eosinophils, which drives phagocytosis and degranulation, respectively. It can also fix complement via an unknown alternative pathway. IgM is expressed as either a pentamer or a hexamer, both of which are held together by a J-chain. IgM has a serum half-life of 5 days. It binds weakly to Clq via a binding site located in its CH3 domain. IgD has a half-life of 3 days in serum. It is unclear what effect or functions are attributable to this Ig. IgE is a monomeric Ig and has a serum half-life of 2.5 days. IgE binds to two Fc receptors which drives degranulation and results in the release of proinflammatory agents. Depending on the desired effect, the heterologous fusion proteins may contain any of the isotopes described above or may contain mutated Fc regions wherein the complement and/or Fc receptor binding functions have been altered. Thus, the heterologous fusion proteins may contain the entire Fc portion of an immunoglobulin, fragments of the Fc portion of an immunoglobulin, or analogs thereof fused to an exendin, exendin agonist or a GLP-1 receptor agonist compound.

The fusion proteins can consist of single chain proteins or as multi-chain polypeptides. Two or more Fc fusion proteins can be produced such that they interact through disulfide bonds that naturally form between Fc regions. These multimers can be homogeneous with respect to the drug compound or they may contain different drug compounds fused at the N-terminus of the Fc portion of the fusion protein. Regardless of the final structure of the fusion protein, the Fc or Fc-like region must serve to prolong the in vivo plasma half-life of the drug compound fused at the N-terminus. Furthermore, the fused drug compound must retain some biological activity.

Since the Fc region of IgG produced by proteolysis has the same in vivo half-life as the intact IgG molecule and Fab fragments are rapidly degraded, it is believed that the relevant sequence for prolonging half-life reside in the CH2 and/or CH3 domains. Further, it has been shown in the literature that the catabolic rates of IgG variants that do not bind the high-affinity Fe receptor or Clq are indistinguishable from the rate of clearance of the parent wild-type antibody, indicating that the catabolic site is distinct from the sites involved in Fc receptor or Clq binding. (Wawrzynczak et al., (1992) Molecular Immunology 5 29:221). Site-directed mutagenesis studies using a marine IgG1 Fe region suggested that the site of the IgG1 Fc region that controls the catabolic rate is located at the CH2-CH3 domain interface. Based on these studies, Fe regions can be modified at the catabolic site to optimize the half-life of the fusion proteins. In one embodiment the Fe region used for the heterologous fusion proteins can be derived from an IgG1 or an IgG4 Fe region. Yet further the Fe region can be IgG4 or derived from IgG4. Even further the IgG Fc region contains both the CH2 and CH3 regions including the hinge region.

Thus in another embodiment the exendin, exendin agonist or GLP-1 receptor agonist compound can be fused directly or via a peptide linker to albumin or an analog, fragment, or derivative thereof. Generally the albumin proteins making up part of the fusion proteins can be derived from albumin cloned from any species. However, human albumin and fragments and analogs thereof reduce the risk of the fusion protein being immunogenic in humans. Human serum albumin (HSA) consists of a single non-glycosylated polypeptide chain of 585 amino acids with a formula molecular weight of 66,500. The amino acid sequence of human SA is provided below. (See also Meloun, et al. (1975) FEBS Letters 58:136; Behrens, et al. (1975) Fed. Proc. 34:591; Lawn, et al. (1981)Nucleic Acids Research 9:6102-6114; Minghetti, et al. (1986) J. Biol. Chem. 261:6747). A variety of polymorphic variants as well as analogs and fragments of albumin have been described. (See Weitkamp, et al., (1973) Ann. Hum. Genet. 37:219). For example, EP322094 disclose shorter forms of HSA. Thus in some embodiments the albumin comprises any of fragments HSA(1-373), HSA(1-388), HSA(1-389), HSA(1-369), and HSA(1 5 419) and fragments between 1-369 and 1-419. EP 399666 discloses albumin fragments that include HSA(1-177) and HSA(1-200) and fragments between HSA(1-177) and HSA(1-200). It is understood that the heterologous fusion protein is biologically active and has a longer plasma half-life than the unconjugated drug compound alone. Thus, the albumin portion of the fusion protein need not necessarily have a plasma half-life equal to that of native human albumin. Fragments, analogs, and derivatives are known or can be generated that have longer half-lives or have half-lives intermediate to that of native human albumin and the drug compound of interest.

Specific examples of heterologous fusion proteins having biological activity and increased half-life are Val8-GLP-1-HSA, Val8-GLP-1-[Gly-Gly-Gly-Gly-Ser]3-HSA, Exendin-4-HSA and Exendin-4-[Gly-Gly-Gly-Gly-Ser]3-HSA. Further examples are Val8-GLP-1-CEX-IgG1, Val8-Glu9-GLP-1-CEx-IgG1, Exendin-4-C2-IgG1, Exendin-4-Linker-IgG1, Gly-Glu-GLP-1-CEx-Linker-HSA, Gly-Glu-GLP-1-CEx-Linker-IgG4. CEx refers to a C-terminal extension and comprises the sequence of Ser-Ser-Gly-Ala-Pro-Pro-Pro-Ser. Linker in the above conjugates is Gly-Gly-Gly-Gly-Ser-Gly-Gly-Gly-Gly-Ser-Gly-Gly Gly-Gly-Ser; C2 is Ser-Ser-Gly-Ala-Ser-Ser-Gly-Ala. The amino acid sequences of these fusion proteins and their components (e.g. HAS) are: Val8-GLP-1-Human serum albumin amino acid sequence (SEQ ID NO:_), Val-GLP-1-Linker-Human serum albumin amino acid sequence (SEQ ID NO:_), Gly-Glu-GLP-1-CEx-Linker-Human serum albumin amino acid sequence (SEQ ID NO:_), Exendin-4-Human serum albumin amino acid sequence (SEQ ID NO:_), Val8-GLP-1-IgG1 amino acid sequence (SEQ ID NO:_), Val-GLP-1-Cex-IgG1 amino acid sequence (SEQ ID NO:_), and Exendin-4-C2-IgG1 amino acid sequence (SEQ ID NO:_),

In further embodiments, the Fc analogs and fusions contemplated herein in the practice of the methods described herein include without limitation those Fc variants and fusion proteins described in Published Application WO 2004/110472, filed Jun. 10, 2004, entitled “Fusion Proteins,” which application is herein incorporated by reference in its entirety and for all purposes, including for its specific Fc sequences and linkers. As noted above, the Fc can contain substituted amino acids at various positions that lessen or eliminate effecter function and/or do not have glycosylation sites and thus have reduced heterogeneity during expression. Furthermore, the substitutions can be those that do not induce an immune response after repeated and prolonged administration of the heterologous fusion protein. The heterologous fusion proteins can contain an Fe portion which is derived from human IgG4, but comprises one or more substitutions compared to the wild-type human sequence. The IgG4 Fc portion of the heterologous fusion proteins may contain one or more of the following substitutions: substitution of proline for glutamate at residue 233, alanine or valine for phenylalanine at residue 234 and; alanine or glutamate for leucine at residue 235 (KU numbering, Kabat, E. A. et al. (1991) Sequences of Proteins of Immunological Interest, 5th Ed. U.S. Dept. of Health and Human Services, Bethesda, Md., NIH Publication no. 91-3242). These residues corresponds to positions 16, 17 and 18 in the Fc formula provided below. Further, removing the N-linked glycosylation site in the IgG4 Fe region by substituting Ala for Asn at residue 297 (KU numbering) which corresponds to position 80 of the Fc formula is another way to ensure that residual effecter activity is eliminated in the context of a heterologous fusion protein. In addition, the IgG4 Fe portion of the heterologous fusion proteins can contain a substitution that stabilizes heavy chain dimer formation and prevents the formation of half-IgG4 Fe chains. The heterologous fusion proteins can exist as dimers joined together by disulfide bonds and various non-covalent interactions. Wild-type IgG4 contains a Pro-Pro-Cys-Pro-Ser-Cys (SEQ ID NO:_) motif beginning at residue 224 (KU numbering). This motif in a single active therapeutic peptide-Fc chain forms disulfide bonds with the corresponding motif in another active therapeutic to peptide-Fc chain. However, the presence of serine in the motif causes the formation of single chain heterologous fusion proteins. Accordingly, the IgG4 sequence in yet a further embodiment can be modified such that serine at position at 228 (KU numbering) is substituted with proline (amino acid residue 11 in the FC formula below). The C-terminal lysine residue present in the native molecule may be deleted in the IgG4 derivative Fe portion of the heterologous fusion proteins (position 230 of the Fc formula; deleted lysine referred to as des K). Heterologous fusion proteins expressed in some cell types wherein lysine is encoded by the C-terminal codon are heterogeneous in that a portion of the molecules have lysine as the C-terminal amino acid and a portion have lysine deleted. The deletion is due to protease action during expression in some types of mammalian cells. Thus, to avoid this heterogeneity, a heterologous fusion expression constructs can lack a C-terminal codon for lysine.

In vivo function and stability of any of the heterologous fusion proteins described herein can be optimized by adding small peptide linkers to prevent potentially unwanted domain interactions. Further, a glycine rich linker provides some structural flexibility such that the active therapeutic peptide portion can interact productively with its receptor on target cells. These linkers, however, can significantly increase the risk that the heterologous fusion protein will be immunogenic in vivo. Thus, in one embodiment the linker length is no longer than necessary to prevent unwanted domain interactions and/or optimize biological activity and/or stability.

Fe sequences with such properties as described above for use in the heterologous fusion proteins described herein can comprise the formula (SEQ ID NO:_):

Xaa¹-Glu-Ser-Lys-Tyr-Gly-Pro-Pro-Cys-Pro-Pro-Cys-Pro-Ala-Pro Xaa¹⁶-Xaa¹⁷-Xaa¹⁸-
Gly-Gly-Pro-Ser-Val-Phe-Leu-Phe-Pro-Pro-Lys-Pro Lys-Asp-Thr-Leu-Met-IIe-Ser-Arg-
Thr-Pro-Glu-Val-Thr-Cys-Val Val-Val-Asp-Val-Ser-Gln-Glu-Asp-Pro-Glu-Val-Gln-
Phe-Asn-Trp Tyr-Val-Asp-Gly-Val-Glu-Val-His-Asn-Ala-Lys-Thr-Lys-Pro-Arg Glu-
Glu-Gln-Phe-Xaa⁸⁰-Ser-Thr-Tyr-Arg-Val-Val-Ser-Val-Leu-Thr-Val-Leu-His-Gln-Asp-
Trp-Leu-Asn-Gly-Lys-Glu-Tyr-Lys-Cys-Lys Val-Ser-Asn-Lys-Gly-Leu-Pro-Ser-Ser-
Ile-Glu-Lys-Thr-Ile-Ser Lys-Ala-Lys-Gly-Gln-Pro-Arg-Glu-Pro-Gln-Val-Tyr-Thr-Leu-
Pro Pro-Ser-Gln-Glu-Glu-Met-Thr-Lys-Asn-Gln-Val-Ser-Leu-Thr-Cys Leu-Val-Lys-
Gly-Phe-Tyr-Pro-Ser-Asp-IIe-Ala-Val-Glu-Trp-Glu Ser-Asn-Gly-Gln-Pro-Glu-Asn-
Asn-Tyr-Lys-Thr-Thr-Pro-Pro-Val Leu-Asp-Ser-Asp-Gly-Ser-Phe-Phe-Leu-Tyr-Ser-
Arg-Leu-Thr-Val Asp-Lys-Ser-Arg-Trp-Gln-Glu-Gly-Asn-Val-Phe-Ser-Cys-Ser-Val
Met-His-Glu-Ala-Leu-His-Asn-His-Tyr-Thr-Gln-Lys-Ser-Leu-Ser-Leu-Ser-Leu-Gly-
Xaa²³⁰,

wherein Xaa at position 1 is Ala or absent; Xaa at position 16 is Pro or Glu; Xaa at position 17 is Phe, Val, or Ala; Xaa at position 18 is Leu, Glu, or Ala; Xaa at position 80 is Asn or Ala; and; Xaa at position 230 is Lys or is absent.

In one embodiment GLP-1-Fc fusions include the following proteins: Gly8-Glu22-Gly36-GLP-1(7-37)-1L-IgG4 (S228P), Gly8-Glu22-Gly36-GLP-1(7-37)-1L-IgG4 (S228P, F234A, L235A), Gly8-Glu22-Gly36-GLP-1(7-37)-1 L-IgG4 (S228P, N297A), Gly8-Glu22-Gly36-GLP-i(7-37)-1L-IgG4 (S228P, F234A, L235A, N297A), Gly8-Glu22-Gly36-GLP-1 (7-37)-i.5L-IgG4 (S228P), Gly8-Glu22-Gly36-GLP-i(7-37)-1.5L-IgG4 (S228P, F234A, L235A), Gly8-Glu22-Gly36-GLP-1(7-37)-1.5L-IgG4 (S228P, N297A), Gly8-Glu22-Gly36-GLP-1(7-37)-1.5L-IgG4 (S228P, F234A, L235A, N297A), Gly8-Glu22-Gly36-GLP-i(7-37)-2L-IgG4 (S228P), Gly8-Glu22-Gly36-GLP-i(7-37)-2L-IgG4 (S228P, F234A, L235A), Gly8-Glu22-Gly36-GLP-1 (7-37)-2L-IgG4 (S228P, N297A), and Gly8-Glu22-Gly36-GLP-i (7-37)-2L-IgG4 (S228P, F234A, .L235A, N297A), and the Val8 and des-K forms of all of the above. The nomenclature used herein to refer to specific fusion proteins is defined as follows: Specific substitutions to the GLP-1 portion of the fusion protein are indicated using the specific amino acid being substituted followed by the residue number. GLP-1(7-37) indicates that the GLP-1 portion of the mature fusion protein begins with H is at position 7 and ends with Gly at position 37. L refers to a linker with the sequence Gly-Gly-Gly-Gly-Ser-Gly-Gly-Gly-Gly-Ser-Gly-Gly-Gly-Gly-Ser (SEQ ID NO:_). The number immediately preceding the L refers to the number of linkers separating the GLPL 1 portion from the Fc portion. A linker specified as 1.5 L refers to the sequence Gly-Ser-Gly-Gly-Gly-Gly-Ser-Gly-Gly-Gly-Gly-Ser-Gly-Gly-Gly-Gly-Ser-Gly-Gly-Gly-Gly-Ser (SEQ ID NO:_). IgG4 refers to an analog of the human IgG4 Fc sequence specified shown in the above published application as SEQ ID NO. 7. Substitutions in the IgG4 Fc portion of the fusion protein are indicated in parenthesis. The wild-type amino acid is specified by its common abbreviation followed by the position number in the context of the entire IgG4 sequence using the EU numbering system followed by the amino acid being substituted at that position specified by its common abbreviation.

In some embodiments, exendin and exendin agonist and GLP-1 receptor agonist heterologous fusion compounds contemplated in the practice of the methods as described herein include without limitation those prepared as in or described in Published Application WO 2006/068910, filed Dec. 15, 2005, entitled “GLP 1 Analog Fusion Protein Formulations,” which application is herein incorporated by reference in its entirety and for all purposes, including the formulations of Fc containing fusions. As described herein are a more stable solution formulation for heterologous fusions of an Fc macromolecule and an exendin, exendin agonist or GLP-1 receptor agonist described herein. A formulation comprising a therapeutically effective amount of an Fc fusion at a pH between about pH 6 and about pH 8.5, between about pH 6 and about pH 7.5, between about pH 6 and about pH 7, between about pH 6.5 and about pH 7.5, or between about pH 6 and about pH 6.5, and even further about pH 6 or about 6.5, provide greater chemical stability than when compared to an Fc fusion at a pH outside the described ranges. The pH of the Fe fusion formulations is adjusted to provide acceptable stability, to maintain the solubility and bioactivity of the exendin, exendin agonist or GLP-1 receptor agonist Fc-conjugate and be acceptable for parenteral administration. In one embodiment, the pH of the fusion formulation can be adjusted to between about pH 6 and about pH 8.5, between about pH 6 and about pH 7.5, between about pH 6 and about pH 7, between about pH 6.5 and about pH 7.5, or between about pH 6 and about pH 6.5, and in yet further, about pH 6 or about 6.5. The formulations comprising an Fe fusion may optionally encompass a pharmaceutically acceptable buffer. However, the selection and concentration of the buffer shall be such that the formulation can be adjusted to the described ranges that provide acceptable stability and bioactivity. Examples of pharmaceutically acceptable buffers for the Fe fusions include phosphate buffers like dibasic sodium phosphate, TRIS, acetate, such as sodium acetate, citrate, such as sodium citrate, sodium tartarate, basic amino acids such as histidine, lysine or arginine, or neutral amino acids such as glycine and glycyl-glycine. Other pharmaceutically acceptable buffers are known in the art. In a further embodiment the buffer is selected from the group consisting of citrate, phosphate and TRIS. The skilled artisan will recognize that the selection of the buffer is dependent upon the described pH ranges and the pKa of the buffer. In one embodiment, the concentration of a buffer is between about 1 mM and 30 mM. Yet further the concentration is between about 4 mM and 14 mM or between about 5 mM and 20 mM. Yet further the concentration is between about 10 mM and 20 mM. Even further the concentration is about 10 mM or about 20 mM. The Fc fusion formulations may optionally encompass a preservative. However, the selection and concentration of the preservative shall be such that the formulation can be adjusted to the described ranges that provide acceptable stability and bioactivity. Among preservatives known in the art as being effective and acceptable in parenteral formulations are phenolic preservatives, alkylparabens, benzyl alcohol, chlorobutanol, resorcinol, and other similar preservatives, and various mixtures thereof. Examples of phenolic derivatives include cresols and phenol or a mixture of cresols and phenol. Examples of cresols include meta-cresol, ortho-cresol, para-cresol, chloro-cresol, or mixtures thereof. Alkylparaben refers to a C1 to C4 alkyl paraben, or mixtures thereof. Examples of alkylparabens include methylparaben, ethylparaben, propylparaben, or butylparaben. The concentration of the preservative is known to one skilled in the art. The concentrations must be sufficient to maintain preservative effectiveness by retarding microbial growth. In one embodiment the preservative is meta-cresol or phenol. In general, the concentration of meta-cresol is between about 2.0 to about 8.0 mg/mL, about 2.5 mg/mL to about 4.5 mg/mL, and about 2.0 mg/mL to about 4.0 mg/mL. Further, the concentration of preservative in the Fe formulation is about 2.7 mg/mL. In another embodiment, the concentration of phenol is between about 2.0 to about 10.0 mg/mL, and about 4.0 to about 8.0 mg/mL. Even further the preservative in the formulation is about 5.0 mg/mL. The Fc fusion formulations may optionally encompass an isotonicity agent. However, the selection and concentration of the isotonicity agent shall be such that the formulation can be adjusted to the described ranges that provide acceptable stability and bioactivity. Isotonicity agents refer to compounds that are tolerated physiologically and impart a suitable tonicity to the formulation to prevent the net flow of water across cell membranes. Examples of such compounds include glycerin (or glycerol), salts, e.g., NaCl, and sugars, e.g., dextrose, mannitol, and sucrose. These compounds are commonly used for such purposes at known concentrations. One or more isotonicity agents may be added to adjust the ionic strength or tonicity. In one embodiment the isotonicity agent is NaCl. The concentration of NaCl can be between about 10 mM and 500 mM, between about 50 mM and 200 mM, and even further is about 150 mM. In another embodiment, the isotonicity agent is mannitol. The concentration of the mannitol can be between about 1% (weight (w)/volume (v)) and 10% (w/v), and further is between about 2% (w/v) and 8% (w/v). In another embodiment, the isotonicity agent is glycerin. The concentration of the glycerin can be between about 12 mg/mL and 25 mg/mL, between about 12 mg/mL and 20 mg/mL, and further is about 17 mg/ml. The formulations may optionally encompass a solubility enhancer. However, the selection and concentration of the solubility enhancer shall be such that the Fc fusion formulation can be adjusted to the described ranges that provide acceptable stability and bioactivity. Solubility enhancers provide stability such that the Fc fusion remains soluble for an extended period of time under the conditions of storage. In one embodiment the solubility enhancer is nicotinamide. In general, the concentration of nicotinamide is between 0.01 and 2 molar. Other ranges of nicotinamide concentration are: between 0.05 molar and 1.5 molar; between 0.1 molar and 1.0 molar; between 0.1 molar and 0.5 molar; between 0.5 molar and 1.0 molar; and between 0.15 molar and 0.25 molar. Other additives, such as a pharmaceutically acceptable solubilizers like Tween 20® (polyoxyethylene (20) sorbitan monolaurate), Tween 40® (polyoxyethylene (20) sorbitan monopalmitate), Tween 80® (polyoxyethylene (20) sorbitan monooleate), Pluronic F68® (polyoxyethylene polyoxypropylene block copolymers), and PEG (polyethylene glycol) may optionally be added to the formulation. In one embodiment the solubilizer is Tween 20® or Tween80®. In general, the concentration of Tween 20® or Tween8O® is between 0.001% and 0.05%, between 0.005% and 0.05%; between 0.0075% and 0.05%; and between 0.01% and 0.05%.

Accordingly, a stable solution Fc formulation can comprise a therapeutically effective amount of an exendin-, exendin agonist- or a GLP-1 receptor agonist-Fc fusion at a pH of between about pH 6 and about pH 8.5 wherein the Fc fusion comprises an Fe portion of an immunoglobulin comprising the sequence (SEQ ID NO:_):

Ala-Glu-Ser-Lys-Tyr-Gly-Pro-Pro-Cys-Pro-Pro-Cys-Pro-Ala-Pro-Xaa16-Xaa17-Xaa18-
Gly-Gly-Pro-Ser-Val-Phe-Leu-Phe-Pro-Pro-Lys-Pro-Lys-Asp-Thr-Leu-Met-Ile-Ser-
Arg-Thr-Pro-Glu-Val-Thr-Cys-Val-Val-Val-Asp-Val-Ser-Gln-Glu-Asp-Pro-Glu-Val-
Gln-Phe-Asn-Trp-Tyr-Val-Asp-Gly-Val-Glu-Val-His-Asn-Ala-Lys-Thr-Lys-Pro-Arg-
Glu-Glu-Gln-Phe-Xaa80-Ser-Thr-Tyr-Arg-Val-Val-Ser-Val-Leu-Thr-Val-Leu-His-Gln-
Asp-Trp-Leu-Asn-Gly-Lys-Glu-Tyr-Lys-Cys-Lys-Val-Ser-Asn-Lys-Gly-Leu-Pro-Ser-
Ser-Ile-Glu-Lys-Thr-Ile-Ser-Lys-Ala-Lys-Gly-Gln-Pro-Arg-Glu-Pro-Gln-Val-Tyr-Thr-
Leu-Pro-Pro-Ser-Gln-Glu-Glu-Met-Thr-Lys-Asn-Gln-Val-Ser-Leu-Thr-Cys-Leu-Val-
Lys-Gly-Phe-Tyr-Pro-Ser-Asp-Ile-Ala-Val-Glu-Trp-Glu-Ser-Asn-Gly-Gln-Pro-Glu-
Asn-Asn-Tyr-Lys-Thr-Thr-Pro-Pro-Val-Leu-Asp-Ser-Asp-Gly-Ser-Phe-Phe-Leu-Tyr-
Ser-Arg-Leu-Thr-Val-Asp-Lys-Ser-Arg-Trp-Gln-Glu-Gly-Asn-Val-Phe-Ser-Cys-Ser-
Val-Met-His-Glu-Ala-Leu-His-Asn-His-Tyr-Thr-Gln-Lys-Ser-Leu-Ser-Leu-Ser-Leu-
Gly-Xaa230,

wherein Xaa at position 16 is Pro or Glu; Xaa at position 17 is Phe, Val, or Ala; Xaa at position 18 is Leu, Glu, or Ala; Xaa at position 80 is Asn or Ala; and Xaa at position 230 is Lys or is absent. Even further the stable solution Fc formulation is between about pH 6 and about pH 7.5. Yet further the stable solution Fc formulation is between about pH 6 and about pH 7, is between about pH 6 and about pH 6.5, is about pH 6 or is about pH 6.5. In one embodiment the Fe formulation further comprises Tween 20® and/or Tween 80, and NaCl, and m-Cresol.

In a further embodiment the compound fused to the Fe, optionally via linker, comprises a GLP-1 analog comprising a sequence selected from the group consisting of:
a) His-Xaa⁸-Glu-Gly-Thr-Phe-Thr-Ser-Asp-Val-Ser-Ser-Tyr-Leu-Glu-Glu-Gln-Ala-Ala-Lys-Glu-Phe-Ile-Ala-Trp-Leu-Val-Lys-Gly-Gly-Gly (SEQ ID NO:_); wherein Xaa⁸ is selected from Gly and Val;
b) His-Xaa⁸-Glu-Gly-Thr-Phe-Thr-Ser-Asp-Val-Ser-Ser-Tyr-Leu-Glu-Glu-Gln-Ala-Ala-Lys-Glu-Phe-Ile-Ala-Trp-Leu-Lys-Asn-Gly-Gly-Gly (SEQ ID NO:_) wherein Xaa⁸ is selected from Gly and Val;
c) His-Xaa⁸-Glu-Gly-Thr-Phe-Thr-Ser-Asp-Val-Ser-Ser-Tyr-Leu-Glu-Glu-Gln-Ala-Ala-Lys-Glu-Phe-Ile-Ala-Trp-Leu-Val-Lys-Gly-Gly-Pro (SEQ ID NO:_) wherein Xaa⁸ is selected from Gly and Val;
d) His-Xaas-Glu-Gly-Thr-Phe-Thr-Ser-Asp-Val-Ser-Ser-Tyr-Leu-Glu-Glu-Gln-Ala-Ala-Lys-Glu-Phe-Ile-Ala-Trp-Leu-Lys-Asn-Gly-Gly-Pro (SEQ ID NO:_) wherein Xaa⁸ is selected from Gly and Val;
e) His-Xaa⁸-Glu-Gly-Thr-Phe-Thr-Ser-Asp-Val-Ser-Ser-Tyr-Leu-Glu-Glu-Gln-Ala-Ala-Lys-Glu-Phe-Ile-Ala-Trp-Leu-Val-Lys-Gly-Gly (SEQ ID NO:_) wherein Xaa⁸ is selected from Gly and Val; and
f) His-Xaa⁸-Glu-Gly-Thr-Phe-Thr-Ser-Asp-Val-Ser-Ser-Tyr-Leu-Glu-Glu-Gln-Ala-Ala-Lys-Glu-Phe-Ile-Ala-Trp-Leu-Lys-Asn-Gly-Gly (SEQ ID NO:_) wherein Xaa⁸ is selected from Gly and Val.

### DEFINITIONS

In accordance with the present invention and as used herein, the following terms are defined to have the following meanings, unless explicitly stated otherwise.

The term amino acid refers to natural amino acids, unnatural amino acids, and amino acid analogs, all in their D and L stereoisomers if their structure allow such stereoisomeric forms. Natural amino acids include alanine (Ala), arginine (Arg), asparagine (Asn), aspartic acid (Asp), cysteine (Cys), glutamine (Gln), glutamic acid (Glu), glycine (Gly), histidine (H is), isoleucine (Ile), leucine (Leu), Lysine (Lys), methionine Net), phenylalanine (Phe), proline (Pro), serine (Ser), threonine (Thr), typtophan (Trp), tyrosine (Tyr) and valine (Val). Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic, acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid and thioproline. Amino acid analogs include the natural and unnatural amino acids which are chemically blocked, reversibly or irreversibly, or modified on their N-terminal amino group or their side-chain groups, as for example, methionine sulfoxide, methionine sulfone, S-(carboxymethyl)-cysteine, S-(carboxymethyl)-cysteine sulfoxide and S-(carboxymethyl)-cysteine sulfone.

The term amino acid analog refers to an amino acid wherein either the C-terminal carboxy group, the N-terminal amino group or side-chain functional group has been chemically codified to another functional group. For example, aspartic acid-(beta-methyl ester) is an amino acid analog of aspartic acid; N-ethylglycine is an amino acid analog of glycine; or alanine carboxamide is an amino acid analog of alanine.

The term amino acid residue refers to radicals having the structure: (1) —C(O)—R—NH—, wherein R typically is —CH(R′)—, wherein R′ is an amino acid side chain, typically H or a carbon containing substituent; Or (2)

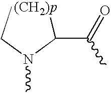

wherein p is 1, 2 or 3 representing the azetidinecarboxylic acid, proline or pipecolic acid residues, respectively.

The term lower referred to herein in connection with organic radicals such as alkyl groups defines such groups with up to and including about 6, preferably up to and including 4 and advantageously one or two carbon atoms. Such groups may be straight chain or branched chain.

Pharmaceutically acceptable salt includes salts of the compounds described herein derived from the combination of such compounds and an organic or inorganic acid. In practice, the use of the salt form amounts to use of the base form. The compounds are useful in both free base and salt form.

In addition, the following abbreviations stand for the following:
“ACN” or “CH₃CN” refers to acetonitrile;
“Boc”, “tBoc” or “Thoc” refers to t-butoxy carbonyl;
“DCC” refers to N,N′-dicyclohexylcarbodiimide;
“Fmoc” refers to fluorenylmethoxycarbonyl;
“HBTU” refers to 2-(1H-benzotriazol-1-yl)-1,1,3,3,-tetramethyluronium hexafluorophosphate;
“HOBt” refers to 1-hydroxybenzotriazole monohydrate;
“homoP” or hpro” refers to homoproline;
“MeAla” or “Nme” refers to N-methylalanine;
“naph” refers to naphthylalanine;
“pG” or pGly” refers to pentylglycine;
“tBuG” refers to tertiary-butylglycine;
“ThioP” or tPro” refers to thioproline;
“3Hyp” refers to 3-hydroxyproline;
“4Hyp” refers to 4-hydroxyproline;
NAG” refers to N-alkylglycine;
NAPG” refers to N-alkylpentylglycine;
“Norval” refers to norvaline;
“Norleu” refers to norleucine.

### Preparation of Compounds

The exendin, exendin agonist or GLP-1 receptor agonist described herein may be prepared using standard solid-phase peptide synthesis techniques and preferably an automated or semiautomated peptide synthesizer. The exendin, exendin agonist or GLP-1 receptor agonist may be prepared using any method known by one skilled in the art, including those described in U.S. application Ser. No. 09/756,690, “Use of Exendins and Agonists Thereof For Modulation of Triglyceride Levels and Treatment of Dyslipidemia” which was filed Jan. 9, 2001, and which is herein incorporated by reference in its entirety. In one embodiment, an α-N-carbamoyl protected amino acid and an amino acid attached to the growing peptide chain on a resin are coupled at room temperature in an inert solvent such as dimethylformamide, N-methylpyrrolidinone or methylene chloride in the presence of coupling agents such as dicyclohexylcarbodiimide and 1-hydroxybenzotriazole in the presence of a base such as diisopropylethylamine. The α-N-carbamoyl protecting group is removed from the resulting peptide-resin using a reagent such as trifluoroacetic acid or piperidine, and the coupling reaction repeated with the next desired N-protected amino acid to be added to the peptide chain. Suitable N-protecting groups are well known in the art, with t-butyloxycarbonyl (tBoc) and fluorenylmethoxycarbonyl (Fmoc) being preferred herein.

The solvents, amino acid derivatives, and 4-methylbenzhydryl-amine resin used in the peptide synthesizer may be purchased for example from Applied Biosystems Inc. (Foster City, Calif.). The following side-chain protected amino acids may be purchased from Applied Biosystems, Inc.: Boc-Arg(Mts), Fmoc-Arg(Pmc), Boc-Thr(Bzl), Fmoc-Thr(t-Bu), Boc-Ser(Bzl), Fmoc-Ser(t-Bu), Boc-Tyr(BrZ), Fmoc-Tyr(t-Bu), Boc-Lys(Cl-Z), Fmoc-Lys(Boc), Boc-Glu(Bzl), Fmoc-Glu(t-Bu), Fmoc-His(Trt), Fmoc-Asn(Trt), and Fmoc-Gln(Trt). Boc-H is(BOM) may be purchased from Applied Biosystems, Inc. or Bachem Inc. (Torrance, Calif.). Anisole, dimethylsulfide, phenol, ethanedithiol, and thioanisole may be obtained from Aldrich Chemical Company (Milwaukee, Wis.). Air Products and Chemicals (Allentown, Pa.) supplies HF. Ethyl ether, acetic acid and methanol may be purchased from Fisher Scientific (Pittsburgh, Pa.).

Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (see, Applied Biosystems User's Manual for the ABI 430A Peptide Synthesizer, Version 1.3B Jul. 1, 1988, section 6, pp. 49-70, Applied Biosystems, Inc., Foster City, Calif.) with capping. Boc-peptide-resins may be cleaved with HF (−5° C. to 0° C., 1 hour). The peptide may be extracted from the resin with alternating water and acetic acid, and the filtrates lyophilized. The Fmoc-peptide resins may be cleaved according to standard methods (Introduction to Cleavage Techniques, Applied Biosystems, Inc., 1990, pp. 6-12). Peptides may be also be assembled using an Advanced Chem Tech Synthesizer (Model MPS 350, Louisville, Ky.).

Peptides may be purified by RP-HPLC (preparative and analytical) using a Waters Delta Prep 3000 system. A C4, C8 or C18 preparative column (10μ, 2.2×25 cm; Vydac, Hesperia, Calif.) may be used to isolate peptides, and purity may be determined using a C4, C8 or C18 analytical column (5μ, 0.46×25 cm; Vydac). Solvents (A=0.1% TFA/water and B=0.1% TFA/CH₃CN) may be delivered to the analytical column at a flowrate of 1.0 ml/min and to the preparative column at 15 ml/min. Amino acid analyses may be performed on the Waters Pico Tag system and processed using the Maxima program. Peptides may be hydrolyzed by vapor-phase acid hydrolysis (115° C., 20-24 h). Hydrolysates may be derivatized and analyzed by standard methods (Cohen, et al., The Pico Tag Method: A Manual of Advanced Techniques for Amino Acid Analysis, pp. 11-52, Millipore Corporation, Milford, Mass. (1989)). Fast atom bombardment analysis may be carried out by M-Scan, Incorporated (West Chester, Pa.). Mass calibration may be performed using cesium iodide or cesium iodide/glycerol. Plasma desorption ionization analysis using time of flight detection may be carried out on an Applied Biosystems Bio-Ion 20 mass spectrometer. Electrospray mass spectroscopy may be carried out on a VG-Trio machine.

Peptide compounds useful in the invention may also be prepared using recombinant DNA techniques, using methods known in the art. See, e.g., Sambrook et al., Molecular Cloning: A Laboratory Manual, 2d Ed., Cold Spring Harbor (1989). Non-peptide compounds useful in the present invention may be prepared by art-known methods. For example, phosphate-containing amino acids and peptides containing such amino acids may be prepared using methods known in the art. See, e.g., Bartlett and Landen, Biorg. Chem. 14:356-377 (1986).

In an embodiment, methods of the present invention comprise administering one or more exendin, exendin agonist or GGP-1 receptor agonist. For administration, compositions useful in the invention may conveniently be provided in the form of formulations suitable for parenteral (including intravenous, intramuscular, and subcutaneous) or nasal, buccal or oral administration. In some cases, it will be convenient to provide an exendin, exendin agonist or GLP-1 receptor agonist and another lipid-controlling agent, such as a statin, in a single composition or solution for administration together. Exemplary statins include atorvastatin, fluvastatin, lovastatin, pravastatin, simvastatin and rosuvastatin. In other cases, it may be more advantageous to administer the additional agent separately from the exendin, exendin agonist or GLP-1 receptor agonist. A suitable administration format may best be determined by a medical practitioner for each patient individually. Suitable pharmaceutically acceptable carriers and their formulation are described in standard formulation treatises, e.g., Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2 S (1988).

Compounds useful in the methods described herein can be provided as parenteral compositions for injection or infusion. Exemplary formulations are those described and claimed in U.S. application Ser. No. 60/116,380, “Novel Exendin Agonist Formulations and Methods of Administration Thereof,” which was filed on Jan. 14, 1999 and published as U.S. Patent Publication 20030087820 A1 on May 8, 2003, both of which are herein incorporated by reference in their entireties and for all purposes.

Formulations include, for example, compounds suspended in an inert oil, suitably a vegetable oil such as sesame, peanut, olive oil, or other acceptable carrier. Preferably, they are suspended in an aqueous carrier, for example, in an isotonic buffer solution at a pH of about 3.0 to about 8.0, preferably at a pH of about 3.5 to about 5.0. These compositions may be sterilized by conventional sterilization techniques, or may be sterile filtered. The compositions may contain pharmaceutically acceptable auxiliary substances as required to approximate physiological conditions, such as pH buffering agents. Useful buffers include for example, sodium acetate/acetic acid buffers. Formulations may also include a preservative. An exemplary preservative is m-cresol, preferably 0.3% m-cresol. A form of repository or “depot” slow release preparation may be used so that therapeutically effective amounts of the preparation are delivered into the bloodstream over many hours or days following transdermal injection or delivery.

In certain embodiments of any of the methods described herein, the peptide or analog thereof can be administered in a polymer-based sustained release device. Such polymer-based sustained release devices are described, for example, in Published Application WO2005/102293A1 and U.S. Patent Publication 2005/0271702, for example, which are incorporated herein by reference in their entireties and for all purposes. Accordingly, in one embodiment, the exendin, exendin agonist and GLP-1 receptor agonist, which are biologically active polypeptides, can be formulated as a composition for the sustained release of the biologically active polypeptide comprising a biocompatible polymer, the biologically active polypeptide and a sugar, wherein the ratio of serum Cₘₐₓ to Cₐᵥₑ is about 3 or less, which avoids an undesirable initial release of peptide after injection that leads to nausea in the subject. In certain embodiments, the sustained release composition comprises the polypeptide from about 0.1% w/w to about 10% w/w of the total weight of the sustained release composition. In certain embodiments, the sustained release composition comprises the polypeptide from about 0.5% w/w to about 5% w/w of the total weight of the sustained release composition. The sustained release composition can comprise a total pore volume of the composition from about 0.1 mL/g or less as determined using mercury intrusion porosimetry, as known in the art.

The sustained release composition can comprise the sugar present from about 0.01% w/w to about 10% w/w of the total weight of the sustained release composition. The sugar can be present from about 0.1% w/w to about 5% w/w of the total weight of the sustained release composition. The sugar can be selected from a monosaccharide, a disaccharide, a sugar alcohol or a combination thereof. In certain embodiments, the sugar is selected from sucrose, mannitol and combinations thereof. The biocompatible polymer can be selected from the group consisting of poly(lactides), poly(glycolides), poly(lactide-co-glycolides), poly(lactic acids), poly(glycolic acids), poly(lactic acid co-glycolic acids), polycaprolactone, polycarbonates, polyesteramides, polyanhydrides, poly(amino acids), polyorthoesters, polycyanoacrylates, poly(p dioxanone), poly(alkylene oxalate)s, biodegradable polyurethanes, blends thereof and copolymers thereof. In further embodiments the polymer comprises poly(lactide-co-glycolide), and further can be a 50:50 poly(lactide-co-glycolide). In certain embodiments the sustained release composition comprises, or in other embodiments consists essentially of, a biocompatible polymer having dispersed therein an exendin, exendin agonist or a GLP-1 receptor agonist, or can be exendin-4, at about 3% w/w or more and sucrose at about 2% w/w or more of the weight of the composition. In such embodiments the biocompatible polymer can be without limitation a poly(lactide-co-glycolide), and further the ratio of serum Cₘₐₓ to Cₐᵥₑ is about 3 or less, and the total pore volume of the composition is about 0.1 mL/g or less as determined using mercury intrusion porosimetry, as known in the art. Accordingly, in certain embodiments of the methods described herein there is provided a composition for the sustained release of the biologically active polypeptide comprising a 50:50 DL PLG 4A polymer, about 3 to 5% (w/w) exendin-4, and about 2% (w/w) sucrose, wherein the ratio of serum Cₘₐₓ to Cₐᵥₑ is about 3 or less and the total pore volume of the composition is about 0.1 mL/g or less. An injectable composition suitable for passage through a 25 gauge needle can comprise a sustained release composition comprising a 50:50 DL PLG 4A polymer, about 3 to 5% (w/w) exendin-4, and about 2% (w/w) sucrose, wherein the ratio of serum Cₘₐₓ to Cₐᵥₑ is about 3 or less and the total pore volume of the composition is about 0.1 mL/g or less, suspended in an injection vehicle comprising sodium carboxymethylcellulose at 3.0% (w/v), sodium chloride at 0.9% (w/v), and Polysorbate 20, NF (Tween 20) at 0.1% (v/v) in water.

The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions.

Compositions can also be formulated as pharmaceutically acceptable salts (e.g., acid addition salts) and/or complexes thereof. Pharmaceutically acceptable salts are non-toxic salts at the concentration at which they are administered. The preparation of such salts can facilitate the pharmacological use by altering the physical-chemical characteristics of the composition without preventing the composition from exerting its physiological effect. Examples of useful alterations in physical properties include lowering the melting point to facilitate transmucosal administration and increasing the solubility to facilitate the administration of higher concentrations of the drug.

Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Acetate salts are preferred. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Carriers or excipients can also be used to facilitate administration of the compound. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents. The compositions or pharmaceutical compositions can be administered by different routes including intravenously, intraperitoneal, subcutaneous, and intramuscular, orally, topically, transmucosally, or by pulmonary inhalation. Exemplary methods of administration are discussed herein as well as in U.S. Application No. 60/116,380, “Novel Exendin Agonist Formulations and Methods of Administration Thereof,” which was filed on Jan. 14, 1999 and published as US patent publication 20030087820 A1 on May 8, 2003, both of which are herein incorporated by reference in their entirety and for all purposes. Suitable excipients may be carrier molecules that include large, slowly metabolized macromolecules such as proteins, polysaccharides, polylactic acids, polyglycolic acids, polymeric amino acids, amino acid copolymers, and inactive virus particles. Liposomes are also included within the definition of pharmaceutically acceptable excipients.

If desired, solutions of the above compositions may be thickened with a thickening agent such as methylcellulose. They may be prepared in emulsified form, either water in oil or oil in water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, e.g., a Triton).

Compositions useful in the invention are prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

For use by the physician, the compositions will be provided in dosage unit form containing an amount of an exendin, exendin agonist or GLP-1 receptor agonist, for example, exendin-3, and/or exendin-4, with or without another small LDL, very small LDL, total LDL-lowering or large LDL, large HDL, or total HDL level-increasing agent. The term “effective amount refers to an amount of a pharmaceutical agent used to treat, ameliorate, prevent, or eliminate the identified condition (e.g., disease or disorder), or to exhibit a detectable therapeutic or preventative effect. The effect can be detected by, for example, chemical markers, antigen levels, or time to a measurable event, such as morbidity or mortality. Therapeutic effects include, for example, shifting lipid particle concentration from small LDL particles to large LDL particles, HDL particles, or both, increasing the concentration of large LDL, large HDL, total HDL, or any combination of said lipoproteins, decreasing the concentration of small LDL, decreasing the concentration of small LDL, very small LDL, total LDL or any combination of said lipoproteins, and increasing the particle size of LDL or HDL is increased in said subject. Effective amounts for a given situation can be determined by routine experimentation that is within the skill and judgment of the clinician.

For an exendin, exendin agonist or GLP-1 receptor agonist, the effective amount can be estimated initially either in cell culture assays, e.g., in animal models, such as rat or mouse models. An animal model may also be used to determine the appropriate concentration range and route of administration. Such information can then be used to determine useful doses and routes for administration in humans.

Efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies may be used in formulating a range of dosage for human use. The dosage contained in such compositions is preferably within a range of circulating concentrations that include an ED50 with little or no toxicity. The dosage may vary within this range depending upon the dosage form employed, sensitivity of the patient, and the route of administration.

In an embodiment, therapeutically effective amounts of an exendin, exendin agonist or GLP-1 receptor agonist for use in treating a subject in need, such as for example a subject with a decreased level of large LDL, large HDL, total HDL level, or any combination thereof include those that increase large LDL, large HDL, total HDL concentration or any combination thereof. In another embodiment, therapeutically effective amounts of an exendin, exendin agonist or GLP-1 receptor agonist for treating a subject in need, for example a subject with an elevated level of small LDL, very small LDL, total LDL or any combination thereof include those that decrease small LDL, very small LDL, total LDL or any combination thereof. As will be recognized by those in the field, an effective amount of therapeutic agent will vary with many factors including the age, weight and health of the patient, the nature and extent of the condition, the patient's physical condition, the blood concentration of one or more lipoproteins, the therapeutic or combination of therapeutics selected for administration and other factors.

The therapeutically effective daily plasma dose of the exendin, exendin agonist or GLP-1 receptor agonist compounds may typically be in the range of from about 0.5 μg to about 3 μg, about 20 μg to about 30 μg, about 0.5 μg to about 1 mg/day, about 1 μg to about 1 mg/day, and, more specifically, from about 1 μg to about 20 μg or from about 1 μg to about 500 μg/day for a 70 kg patient, administered in a single or divided doses. Still more specifically, the therapeutically effective daily plasma lipoprotein controlling dose of the compounds will typically be in the range of from about 1 μg to about 100 μg/day and, more specifically 1 μg to about 50 μg, about 1 μg to about 3 μg to about 20 μg to about 50 μg/day, for a 70 kg patient, administered in a single or divided doses. It is intended that the use of a 70 kg patient is for exemplary purposes and that the above doses can be converted to a per kg basis for administration to a patient having a body weight of greater than or less that 70 kg.

Various exemplary dosages are described in U.S. Application Ser. No. 60/116,380, entitled, “Novel Exendin Agonist Formulations and Methods of Administration Thereof,” which was filed on Jan. 14, 1999 and published as US patent publication 20030087820 A1 on May 8, 2003, both of which are herein incorporated by reference in their entirety and for all purposes.

Exemplary doses for twice daily administration include about 0.01 to 0.3 μg per kilogram, about 0.01 to 0.05 μg per kilogram, and about 0.1 to 0.3 μg per kilogram. Other exemplary doses based upon patient weight for compounds having approximately the potency of exendin-4 range from 0.005 μg/kg per dose to about 0.2 μg/kg per dose. More preferably, doses based upon patient weight for compounds having approximately the potency of exendin-4 range from 0.02 μg/kg per dose to about 0.1 μg/kg per dose. Most preferably, doses based upon patient weight for compounds having approximately the potency of exendin-4 range from 0.05 μg/kg per dose to about 0.1 μg/kg per dose. These doses are administered from 1 to 4 times per day, preferably from 1 to 2 times per day.

In certain embodiments, one or more exendins, exendin agonists or GLP-1 receptor agonists may be administered as long-acting formulations, such as for example a formulation for once or twice weekly administration. Any long-acting formulation known to the skilled artisan may be administered. Exemplary, non-limiting long-acting formulations include those as provided in International Application Nos. PCT/US2004/011547, filed Apr. 15, 2004, and PCT/US2005/012989, filed Apr. 15, 2005 (WO2005102293), and corresponding U.S. Published Applications 20050271702 and 20050271702, each of which are herein incorporated by reference in their entireties. Doses of exendin, exendin agonist or GLP-1 receptor agonist will normally be less if given by continuous infusion.

The exact dose to be administered is determined by the attending clinician and is dependent upon where the particular compound lies within the above quoted range, as well as upon the age, weight and condition of the individual, and the mode of administration. Administration should begin shortly after diagnosis of a need, including for example elevated small LDL, very small LDL, total LDL or decreased large LDL, large HDL, or total HDL concentration, or any combinations thereof and continue until a desired effect is achieved, such a for example a desired lipoprotein concentration is reached or while a desired concentration is maintained.

Administration may be by injection, preferably subcutaneous or intramuscular. Administration may also be by non-injectable routes, for example, via the respiratory tract, the mouth, and the gut. Orally active compounds may be taken orally, however dosages should be increased 5-10 fold. Solid dosage forms, such as those useful for oral, buccal, sublingual, intra-tracheal, nasal or pulmonary delivery may be used. Additionally, preserved or unpreserved liquid formulations or dry powder may be used.

The optimal formulation and mode of administration of compounds of the present application to a patient depend on factors known in the art such as the disease or disorder, the desired effect, and the type of patient. In certain embodiments, the exendin or exendin agonist or GLP-1 receptor agonist is administered independent of the timing of a meal or not for the purpose of controlling prandial, post-prandial or fasting blood glucose levels.

To assist in understanding the present invention, the following Examples are included. The experiments relating to this invention should not, of course, be construed as limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof. The examples illustrate the preparation of the present hybrid polypeptides, and the testing of these hybrid polypeptides of the invention in vitro and/or in vivo. Those of skill in the art will understand that the techniques described in these examples represent techniques described by the inventors to function well in the practice of the invention, and as such constitute exemplary modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

### Administration of Exendin

Fifty two patients participated in a test meal study which was part of a 1-year, 13-country, open-label trial comparing exenatide (exendin-4) and biphasic insulin aspart 30/70 (n=505). Patients with type 2 diabetes inadequately controlled by sulphonylurea and metformin were randomized to exenatide (n=30) or biphasic insulin aspart (n=22). For test meal data, postprandial glucose incremental AUC₀₋₄ₕ is calculated at baseline and week 52. Patients received 5 μg of exenatide BID for a 4-week treatment initiation period, followed by 48 weeks of exenatide therapy at 10 μg BID.

Traditional and nontraditional cardiovascular (CV) markers, including precursor brain natriuretic protein, oxidized LDL-C, highly sensitive C-reactive protein, and apolipoproteins A1 and B, are measured during the fasting state using standard assays.

Changes in lipoprotein particle size and concentration from baseline to endpoint are shown in Table 1.

TABLE 1

| | Exenatide Δ | | Insulin Δ | | Between-Group |
|---|---|---|---|---|---|
| | mean (SEM) | | mean (SEM) | | Difference |
| LDL Particle Size (nm) | +0.33 | (0.10) p = .001 | +0.11 | (0.12) p = .356 | p = .144 |
| HDL Particle Size (nm) | +0.11 | (0.04) p = .013 | +0.04 | (0.05) p = .391 | p = .318 |
| VLDL Particle Size (nm) | −3.2 | (1.9) p = .097 | −1.6 | (2.2) p = .482 | p = .590 |
| Total LDL (nmol/L) | −87.3 | (62.2) p = .169 | +5.0 | (75) p = .947 | p = .348 |
| Large LDL (nmol/L) | +68.4 | (27.3) p = .017 | +37.7 | (32.8) p = .258 | p = .479 |
| Small LDL (nmol/L) | −146.4 | (67.7) p = .037 | −25.8 | (81.2) p = .753 | p = .262 |
| Very Small LDL (nmol/L) | −126.8 | (53.9) p = .024 | −17.0 | (64.6) p = .794 | p = .201 |
| Total HDL (μmol/L) | +0.33 | (0.79) p = .678 | +2.5 | (0.9) p = .012 | p = .088 |
| Large HDL (μmol/L) | +0.69 | (0.33) p = .042 | +1.0 | (0.4) p = .014 | p = .528 |
| Total VLDL (nmol/L) | −9.3 | (5.5) p = .097 | +12.9 | (6.6) p = .056 | p = .014 |
| Small VLDL (nmol/L) | −2.1 | (3.4) p = .547 | +8.5 | (4.2) p = .049 | p = .064 |

### Example 2

### Measurement of Lipoprotein Concentration and Particle Diameter

Lipoprotein subclass particle concentrations and average particle diameters are measured using proton nuclear magnetic resonance spectroscopy. See e.g., Festa et at., Nuclear Magnetic Resonance Lipoprotein Abnormalities in Prediabetic Subjects in the Insulin Resistance Artherosclerosis Study, Circulation, 111:3465-3472 (2005).

The particle concentrations of different lipoprotein subclasses are obtained by measuring the amplitudes of the characteristic lipid methyl NMR signals. The signal amplitudes of various subclasses are extracted from the overall lipid methyl group signal with a spectral deconvolution algorithm (Lipo Science, Inc.). See e.g., Festa. VLDL and LDL particle concentrations are given in nanomoles per liter. HDL particle concentrations are provided in micromoles per liter.

Average particle sizes (in nanometers diameter) are computed as the sum of the diameter of the particle subclass multiplied by the relative mass percentage of the subclass as estimated from the amplitude of its methyl NMR signal.

Particle concentrations and sizes are compared before and after administration of an exendin or exendin agonist. See e.g., Table 1.

### Example 3

### Measurement of Fibrinogen Concentration

Fibrinogen activity is measured by the von Clauss clotting method. By the von Clauss clotting method, thrombin is added to a test sample and the time until clotting is measured. Fibrinogen concentration is determined by comparing clotting time for the test sample to the clotting time of a standard curve for various reference plasmas.

Fibrinogen is measured at baseline, Week 16, Week 52, or at early termination of the study.

Exemplary fibrinogen levels (g/L) are provided in Table 2.

TABLE 2

| | Exenatide | | | Insulin Aspart | | |
|---|---|---|---|---|---|---|
| Week | n | Mean | (SEM) | n | Mean | (SEM) |
| Baseline | 18 | 4.54 | (0.28) | 14 | 4.22 | (0.31) |
| Endpoint (LOCF) | 18 | 3.95 | (0.22) | 14 | 4.16 | (0.33) |

LOCF = last post-baseline measurement carried forward.

All patents and other references cited herein are indicative of the level of skill of those skilled in the art to which the references pertain, and are incorporated by reference in their entireties and for all purposes, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms to describe distinct subject matter. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any two different values as the endpoints of a range. Such ranges are also within the scope of the described invention.

1-100. (canceled)

101. A method for
(i) providing an improved cardiovascular risk profile;
(ii) increasing the concentration of large LDL, large HDL, total HDL, or any combination thereof;
(iii) decreasing the concentration of small LDL, very small LDL, total LDL, or any combination thereof; or
(iv) increasing the average particle size of LDL or HDL;
in a subject in need thereof comprising administering to the subject a therapeutically effective amount of a pharmaceutical composition comprising a GLP-1 receptor agonist compound and a pharmaceutically acceptable carrier.

102. The method of claim 101 for providing an improved cardiovascular risk profile in a subject in need thereof.

103. The method of claim 101 for increasing the concentration of large LDL, large HDL, total HDL, or any combination thereof in a subject in need thereof.

104. The method of claim 101 for decreasing the concentration of small LDL, very small LDL, total LDL, or any combination thereof in a subject in need thereof.

105. The method of claim 101 for increasing the average particle size of LDL or HDL in a subject in need thereof.

106. The method of claim 101, wherein the GLP-1 receptor agonist compound is an exendin, an exendin analog, a GLP-1, or a GLP-1 analog.

107. The method of claim 101, wherein the GLP-1 receptor agonist compound is administered parenterally, nasally, bucally, or orally.

108. The method of claim 101, wherein GLP-1 receptor agonist compound is administered by injection.

109. The method of claim 101, wherein the pharmaceutical composition is a sustained release pharmaceutical composition.

110. The method of claim 109, wherein the sustained release pharmaceutical composition comprises the GLP-1 receptor agonist compound, a biocompatible polymer, and a sugar.

111. The method of claim 109, wherein the sustained release composition consists essentially of a 50:50 □L PLG 4A polymer; about 3 to 5% (w/w) of the GLP-1 receptor agonist compound; and about 2% (w/w) sucrose; wherein the total pore volume of the composition is about 0.1 mL/g or less as determined using mercury intrusion porosimetry; and wherein the plasma Cₘₐₓ to Cₐᵥₑ ratio is about 3 or less.

112. The method of claim 101, wherein the GLP-1 receptor agonist compound is a compound of Formula (I); a compound of Formula (II); a compound of Formula (III); a compound of Formula (IV); a compound of Formula (V); a compound of Formula (VI); a compound of Formula (VII); a compound of Formula (VIII); a compound of Formula (IX); a compound of Formula (X); a compound of Formula (XI); a compound of Formula (XII); exendin-3; exendin-4; exendin-4(1-30); exendin-4(1-27); exendin-4(1-28); ¹⁴Leu, ²⁵Phe-exendin-4; ¹⁴Leu-exendin-4; ¹⁴Leu, ²⁵Phe-exendin-4(1-28); ¹⁴Leu-exendin-4(1-28); [N^{ε}-(17-carboxyheptadecanoic acid)Lys²⁰]exendin-4(1-39)amide; [N^{ε}-(17-carboxy-heptadecanoyl)Lys³²]exendin-4(1-39)amide; exendin-4(1-39)-Lys⁴⁰(ε-MPA)—NH₂; exendin-4(1-39)-Lys⁴⁰(ε-AEEA-AEEA-MPA)-NH₂; GLP-1; GLP-1(7-37); or Arg³⁴Lys²⁶(N^{ε}(γ-glutamyl(N^{α}-decanoyl)))GLP-1(7-37).

113. The method of claim 101, wherein the GLP1 receptor agonist compound is conjugated to at least one macromolecule.

114. The method of claim 113, wherein the macromolecule is a peptide, a blood-protein-binding peptide, a hormone, a polypeptide, an albumin, a polyamino acid, a fatty acyl group, a diacid, a water soluble polymer, an immunoglobulin, an immunoglobulin fragment, a catalytic antibody, or a fragment of a catalytic antibody.

115. The method of claim 113, wherein the GLP-1 receptor agonist compound is conjugated to two macromolecules selected from the group consisting of a peptide, a blood-protein-binding peptide, a hormone, a polypeptide, an albumin, a polyamino acid, a fatty acyl group, a diacid, a water soluble polymer, an immunoglobulin, an immunoglobulin fragment, a

116. A method for
(i) providing an improved cardiovascular risk profile;
(ii) increasing the concentration of large LDL, large HDL, total HDL or any combination thereof;
(iii) decreasing the concentration of small LDL, very small LDL, total LDL, or any combination thereof; or
(iv) increasing the average particle size of LDL or HDL;
in a subject in need thereof comprising administering to the subject a therapeutically effective amount of a particle comprising a GLP-1 receptor agonist compound complexed with a basic polypeptide.

117. The method of claim 116, wherein the basic polypeptide is polylysine, polyarginine, polyornithine, protamine, putrescine, spermine, spermidine, or histone; and wherein the particle comprises the GLP-1 receptor agonist compound and the basic polypeptide in a ratio between about 4:1 and about 10:1; and the mean number diameter of the particle is between 1 μm and 5 μm.

118. A method for treating a cardiovascular disease in a subject in need thereof comprising administering to the subject a therapeutically effective amount of a pharmaceutical composition comprising a GLP-1 receptor agonist compound and a pharmaceutically acceptable carrier.

119. The method of claim 118, wherein the cardiovascular disease is angina, arrhythmia, atrial fibrillation, high blood pressure, high cholesterol, myocardial infarction, heart failure, arteriosclerosis, atherosclerosis, angina, stroke, pericarditis, coronary artery disease, hypertrophic cardiomyopathy, or hyperlipidemia.

120. The method of claim 118, wherein the GLP-1 receptor agonist compound is a compound of Formula (I); a compound of Formula (II); a compound of Formula (III); a compound of Formula (IV); a compound of Formula (V); a compound of Formula (VI); a compound of Formula (VII); a compound of Formula (VIII); a compound of Formula (IX); a compound of Formula (X); a compound of Formula (XI); a compound of Formula (XII); exendin-3; exendin-4; exendin-4(1-30); exendin-4(1-27); exendin-4(1-28); ¹⁴Leu, ²⁵Phe-exendin-4; ¹⁴Leu-exendin-4; ¹⁴Leu, ²⁵Phe-exendin-4(1-28); ¹⁴Leu-exendin-4(1-28); [N^{ε}-(17-carboxyheptadecanoic acid)Lys²⁰]exendin-4(1-39)amide; [N^{ε}-(17-carboxy-heptadecanoyl)Lys³²]exendin-4(1-39)amide; exendin-4(1-39)-Lys⁴⁰(ε-MPA)—NH₂; exendin-4(1-39)-Lys⁴⁰(ε-AEEA-AEEA-MPA)-NH₂; GLP-1; GLP-1(7-37); or Arg³⁴Lys²⁶(N^{ε}(γ-glutamyl(N^{α}-decanoyl)))GLP-1(7-37).
